(19) United States
(12) Patent Application Publication
(10) Pub. No.: US 2026/0220422 A1
(43) Pub. Date: Jul. 30, 2026

(54) METHOD AND SYSTEM FOR PROVIDING GUIDE SUPPORTING PERFORMANCE IMPROVEMENT OF MULTI-TASKING MODEL, METHOD FOR SAMPLING DATA FOR GENERAL-PURPOSE MULTI-TASKING MODEL, AND METHOD AND SYSTEM FOR PROVIDING GENERAL-PURPOSE MULTI-TASKING MODEL INCLUDING SAME

(71) Applicant: LG MANAGEMENT DEVELOPMENT INSTITUTE CO., LTD., Seoul (KR)

(72) Inventors: Dae Woong JEONG, Seoul (KR); Sung Moon KO, Gimpo-si (KR); Su Min LEE, Seoul (KR); Hyun Seung KIM, Seoul (KR); Soo Rin YIM, Seoul (KR)

(21) Appl. No.: 19/630,457

(22) Filed: Mar. 27, 2026

Related U.S. Application Data
(63) Continuation of application No. PCT/KR2025/009693, filed on Jul. 7, 2025.

(30) Foreign Application Priority Data
Aug. 20, 2024 (KR) 10-2024-0111047
Aug. 20, 2024 (KR) 10-2024-0111050

(51) Int. Cl. G06N 3/042; G06N 3/045; G06N 3/0985

(52) U.S. Cl. CPC G06N 3/042; G06N 3/0985; G06N 3/045

(57) ABSTRACT

A method executed by a computer includes: obtaining first input information specifying predetermined domain characteristics; obtaining a validity index that quantitatively indicates difficulty in generating output data of the multi-tasking model according to the first input information; generating first guide information specifying the domain characteristics for reducing the generation difficulty based on the obtained validity index; and providing the first guide information. The method further include: obtaining input information specifying predetermined domain characteristics; converting the input information into low-dimensional latent variables represented in a low-dimensional Gaussian space; obtaining sampled latent variables obtained by sampling the converted low-dimensional latent variables; obtaining optimized latent variables obtained by optimizing the obtained sampled latent variables through a genetic algorithm; restoring the optimized latent variables into high-dimensional latent variables represented in a high-dimensional space; and providing the restored high-dimensional latent variables to the multi-tasking model.

### CROSS REFERENCE TO RELATED APPLICATIONS

This application is a Bypass Continuation of International Patent Application No. PCT/KR2025/009693, filed on Jul. 7, 2025 that claims priority and the benefit of Korean Patent Application No. 10-2024-0111047, filed on Aug. 20, 2024 and Korean Patent Application No. 10-2024-0111050, filed on Aug. 20, 2024, each of which is hereby incorporated by reference for all purposes as if fully set forth herein.

### BACKGROUND

### Field

Embodiments of the invention relate generally to a method and a system for providing a guide supporting performance improvement of a multi-tasking model, and more particularly, to a method and a system for providing a guide for improving the performance of a multi-tasking model, which quantitatively evaluate validity of input data of the multi-tasking model and provide a guide according thereto.

In addition, the present disclosure relates to a method and a system for sampling data for a general-purpose multi-tasking model. More specifically, the present disclosure relates to a method for sampling data for a general-purpose multi-tasking model, which efficiently searches for and optimizes data in a high-dimensional space through data sampling using a low-dimensional space, and a method and a system for providing a general-purpose multi-tasking model including the same.

### Discussion of the Background

The advancement of modern science and technology demands innovative achievements in the fields of molecular design and synthesis. In particular, the ability to accurately predict and design molecular properties is very important for various applications, including drug development, new material research, and/or chemical process optimization.

To meet such demand, machine learning and artificial intelligence techniques have been actively introduced, among which molecular generation models play a key role.

However, several challenges remain in the effective utilization of molecular generation models.

First, the physical and chemical properties of molecules interact with each other, and it is not easy to model complex relationships therebetween. Because these characteristics further hinder model training and prediction performance in high-dimensional data spaces, there is a need to accurately learn molecular properties even in high-dimensional data spaces and to enhance the prediction accuracy and success rate of the models.

Second, there is a need to determine whether the physical property values input by a user are physically valid. Attempting to generate molecules based on abnormal or physically impossible physical property values results in inaccurate model predictions and consequently increases the probability of failure in molecular generation.

In addition, because machine learning models rely heavily on training data, the density and diversity of data directly affect model performance. When training data are concentrated in specific regions or are insufficient, the generalization ability of models may deteriorate and the reliability of predictions may decrease. Therefore, there is a need to solve these problems.

Therefore, there is a need to introduce new techniques that may enhance model prediction performance and secure data diversity, thereby supporting the continuous enhancement of model performance.

In accordance with recent trends of the enlargement and platformization of artificial intelligence models, there is an increasing demand for a single molecular design model that operates universally for large-scale physical properties.

In particular, in the fields of modern chemistry and life science, discovering and designing new materials and molecules are important tasks. In these processes, it is necessary to accurately predict and combine various physical properties of materials. However, because physical properties of materials have high-dimensional and complex correlations, existing methodologies have limitations in effectively designing molecules having desired characteristics.

More specifically, existing molecular generation models may be largely classified into conditional and non-conditional models. Conditional models may design molecules that satisfy specific physical properties, but they require all physical properties to be provided as input values and suffer from difficulties in sampling within a high-dimensional space. Although unconditional models may produce molecules without input conditions, they require repetitive optimization to achieve target physical properties, which results in very high computational costs.

In addition, existing methodologies suffer from the “curse of dimensionality” in a high-dimensional space. This refers to the difficulty of learning and sampling caused by the sparse distribution of data in a high-dimensional space. In particular, the physical properties of molecules are strongly correlated with each other, leaving most of the high-dimensional space empty and causing valid data to be concentrated in very narrow regions.

Therefore, new approaches are required to overcome the limitations of existing methodologies and to efficiently handle large-scale data.

The above information disclosed in this Background section is only for understanding of the background of the inventive concepts, and, therefore, it may contain information that does not constitute prior art.

### SUMMARY

An embodiment of the present disclosure provides a method and a system for providing a guide for improving the performance of a multi-tasking model, which quantitatively evaluate validity of input data of the multi-tasking model and provide a guide according thereto.

In this regard, an embodiment of the present disclosure provides a method and a system for providing a guide for improving the performance of a multi-tasking model, which quantitatively evaluate validity of input data of the multi-tasking model using various deep learning algorithms and provide a visualized guide according thereto.

In addition, an embodiment of the present disclosure provides a method for sampling data for a general-purpose multi-tasking, which efficiently searches for and optimizes data in a high-dimensional space through data sampling using a low-dimensional space, and a method and a system for providing a general-purpose multi-tasking model including the same.

In addition, an embodiment of the present disclosure provides a method and a system for sampling data for a general-purpose multi-tasking model, which execute transfer learning through geometric alignment in an integrated latent space for multi-tasks corresponding to a plurality of domains.

In addition, an embodiment of the present disclosure provides a method and a system for sampling data for a general-purpose multi-tasking model, which simultaneously train various prediction tasks corresponding to a plurality of domains in the transfer learning process to achieve collective learning of not only individual principles of the respective domains but also correlations between the domains and a common principle for the entire domains.

In addition, an embodiment of the present disclosure provides a method and a system for sampling data for a general-purpose multi-tasking model, which implement mutual exchange of information by aligning geometric characteristics of the various prediction tasks.

In addition, an embodiment of the present disclosure provides a method and a system for sampling data for a general-purpose multi-tasking model, which secure data sets for training by utilizing source data from various sources.

In addition, an embodiment of the present disclosure provides a multi-tasking model, which applies the multi-tasking learning model trained as described above to the prediction of relationships between a plurality of physical properties and materials, thereby enabling prediction of a plurality of physical properties for a specific material and prediction of a specific material that satisfies a plurality of physical properties.

However, technical objectives to be achieved by the present disclosure and the embodiments of the present disclosure are not limited to the technical objectives described above, and other technical objectives may exist.

Additional features of the inventive concepts will be set forth in the description which follows, and in part will be apparent from the description, or may be learned by practice of the inventive concepts.

According to one embodiment of the present disclosure, a method executed by a computer includes: receiving, by at least one processor, first input information that specifies predetermined domain characteristics; loading, by the at least one processor, at least one pre-trained artificial intelligence model stored in at least one memory based on the received first input information, the artificial intelligence model being trained using at least one algorithm among at least one density estimation algorithm, at least one anomaly detection algorithm, and at least one similarity determination algorithm; ingesting, by the at least one processor, the first input information into the loaded at least one artificial intelligence model, and generating that quantitatively specifies a difficulty level associated with generating output data of at least one multi-tasking model based on the first input information; generating, by the at least one processor, first guide information that specifies the predetermined domain characteristics configured to reduce the generation difficulty based on the obtained validity index; and manifesting the generated first guide information through at least one interface.

In one implementation, the method may further include: obtaining second input information that specifies the predetermined domain characteristics; obtaining update information that replaces the first input information based on the obtained second input information; and providing the first guide information according to the obtained update information.

In one implementation, the generating of the validity index may include: estimating a density value for the first input information based on a predetermined density estimation algorithm; and calculating the validity index in proportion to the estimated density value.

In one implementation, the generating of the validity index may include: detecting outliers in the first input information based on a predetermined anomaly detection algorithm; estimating a density value for the first input information in inverse proportion to a number of detected outliers; and calculating the validity index in proportion to the estimated density value.

In one implementation of the first aspect, the generating of the validity index may include: calculating a similarity between the output data corresponding to the first input information and actual data based on a predetermined similarity determination algorithm; and calculating the validity index in proportion to the calculated similarity.

In one implementation, the predetermined domain characteristics may include characteristics corresponding to each of a plurality of physical properties.

In one implementation, the first guide information may include a physical property guide that specifies at least one of predetermined physical properties and characteristic values of the physical properties.

In one implementation, the method may further include: generating, by the at least one processor, second guide information that specifies a model training method configured to reduce the generation difficulty based on the obtained validity index; and providing the generated second guide information.

In one implementation, the method may further include providing the second guide information according to the obtained update information.

In one implementation, the obtaining of the second input information may further include providing a data density graph interface that displays, in a graph format, a density value for the first input information derived during specification of the generation difficulty.

In one implementation, the obtaining of the second input information may include: obtaining the second input information in response to a user input made through the provided data density graph interface.

In one implementation of, the obtaining of the second input information may include obtaining the second input information in response to a user that changes a position of a predetermined data point displayed on the data density graph interface.

According to another embodiment of the present disclosure, a system includes: at least one memory; and at least one processor configured to read at least one application stored in the at least one memory and provide the guide supporting performance enhancement of the multi-tasking model. The at least one processor is configured to execute instructions to: receive, by the at least one processor, load, by the at least one processor, at least one pre-trained artificial intelligence model stored in the at least one memory based on the received first input information, the artificial intelligence model being trained using at least one algorithm among at least one density estimation algorithm, at least one anomaly detection algorithm, and at least one similarity determination algorithm; ingest, by the at least one processor, the first input information into the loaded at least one artificial intelligence model, and generate a validity index that quantitatively specifies a difficulty in generating output data of at least one multi-tasking model according to the first input information; generate, by the at least one processor, first guide information that specifies the predetermined domain characteristics configured to reduce the generation difficulty based on the generated validity index; and manifest the generated first guide information through at least one interface.

According to another embodiment of the present disclosure, a method executed by a computer includes: receiving, by at least one processor of the computer, input information specifying predetermined domain characteristics; converting, by the at least one processor, the received input information into low-dimensional latent variables represented in a low-dimensional Gaussian space; obtaining, by the at least one processor, sampled latent variables by sampling the converted low-dimensional latent variables; ingesting, by the at least one processor, the obtained sampled latent variables into at least one artificial intelligence model stored in at least one memory, and obtaining optimized latent variables that are sampled and optimized in the low-dimensional Gaussian space, the artificial intelligence model being trained using at least one genetic algorithm; restoring, by the at least one processor, the obtained optimized latent variables into high-dimensional latent variables represented in a high-dimensional space; ingesting, by the at least one processor, the restored high-dimensional latent variables into at least one multi-tasking model; and manifesting, by the at least one processor, output data of the at least one multi-tasking model through at least one interface.

In another embodiment, the receiving of the input information specifying the domain characteristics may include obtaining N-dimensional input information specifying N domain characteristics, where N satisfies 1≤N<T, wherein the converting of the obtained input information into the low-dimensional latent variables includes converting the obtained N-dimensional input information into low-dimensional latent variables in an L-dimensional Gaussian space, where L satisfies 1≤L<N<T, wherein the restoring of the obtained optimized latent variables into the high-dimensional latent variables includes restoring the L-dimensional optimized latent variables into high-dimensional latent variables in a-dimensional space, where T satisfies T≥2.

In another embodiment, the input information may include physical property input information that is N-dimensional data specifying characteristic values for each of N physical properties, where N satisfies 1≤N<T.

In another embodiment, the obtaining of the sampled latent variables may include sampling the low-dimensional latent variables based on the physical property input information to obtain the sampled latent variables representing a predetermined combination of physical properties.

In another embodiment, the obtaining of the optimized latent variables may include optimizing the sampled latent variables based on the genetic algorithm so as to satisfy the characteristic values for each of the physical properties specified in the physical property input information.

In another embodiment, the obtaining of the optimized latent variables may further include optimizing, among T physical properties included in a T-dimensional physical property combination, where T satisfies T≥2, the N physical properties specified in the physical property input information so as to satisfy the respective characteristic values corresponding thereto.

In another embodiment, the restoring of the obtained optimized latent variables into the high-dimensional latent variables may further include converting the L-dimensional optimized latent variables into the T-dimensional, high-dimensional latent variables while maintaining correlations among the T physical properties.

In another embodiment, the high-dimensional latent variables may represent the physical property combination having a physical feasibility equal to or greater than a predetermined criterion while satisfying the characteristic values for each of the physical properties specified in the physical property input information.

In another embodiment, the ingesting of the high-dimensional latent variables to the at least one multi-tasking model may include providing the high-dimensional latent variables as target characteristics of a prediction process executed by the multi-tasking model, and the target characteristics may include an optimal solution to be achieved by output data of the prediction process executed by the multi-tasking model.

In another embodiment, the manifesting of the output data of the at least one multi-tasking model through at least one interface may include providing a predetermined combination of physical properties according to the prediction process executed in a manner that satisfies the target characteristics, and the prediction process may use the input information as input data, use the predetermined physical property combination according to the input information as output data, and generate the predetermined physical property combination based on geometric alignment in an integrated latent space.

However, effects obtainable in the present disclosure are not limited to the effects mentioned above, and other effects not mentioned may be clearly understood from the following description.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention, and together with the description serve to explain the inventive concepts.

FIG. 1 is a schematic example of a block diagram of a computing system according to an embodiment of the present disclosure.

FIG. 2 is a schematic example of a block diagram of a computing device according to an embodiment of the present disclosure.

FIG. is a schematic example of a block diagram from another perspective of a computing device according to an embodiment of the present disclosure.

FIGS. 4 and 5 are schematic examples of conceptual diagrams for describing a multi-tasking learning model according to an embodiment of the present disclosure.

FIG. 6 is a schematic example of a conceptual diagram of a multi-tasking learning model for predicting a plurality of physical property values for a specific material according to an embodiment of the present disclosure.

FIG. 7 is a schematic internal block diagram of a multi-tasking learning model according to an embodiment of the present disclosure.

FIG. 8 is a schematic example of a conceptual diagram for describing a multi-tasking learning model including a plurality of task processing units according to an embodiment of the present disclosure.

FIG. 9 is a schematic example of a conceptual diagram for describing a multi-tasking model pre-training method according to an embodiment of the present disclosure.

FIG. 10 is a schematic block flowchart for illustrating a multi-tasking model pre-training method according to an embodiment of the present disclosure.

FIG. 11 is a schematic example of a knowledge graph showing relationships among physical properties according to an embodiment of the present disclosure.

FIG. 12 is a schematic block flowchart for describing a multi-tasking learning model training method according to an embodiment of the present disclosure.

FIG. 13 is a schematic example of a first conceptual diagram for describing a multi-tasking learning model training method according to an embodiment of the present disclosure.

FIG. 14 is a schematic example of a second conceptual diagram for describing a multi-tasking learning model training method according to an embodiment of the present disclosure.

FIGS. 15 and 16 are schematic examples of diagrams for describing a regression loss calculation method according to an embodiment of the present disclosure.

FIG. 17 is a schematic example of a diagram for describing an integrated latent space mapping method according to an embodiment of the present disclosure.

FIGS. 18 and 19 are schematic examples of diagrams for describing a consistency loss calculation method according to an embodiment of the present disclosure.

FIGS. 20 and 21 are schematic examples of diagrams for describing a mapping loss calculation method according to an embodiment of the present disclosure.

FIG. 22 is a schematic example of a diagram for describing an integrated loss calculation method according to an embodiment of the present disclosure.

FIG. 23 is a schematic block diagram for describing a method for sampling data for a general-purpose multi-tasking model and a method for providing a general-purpose multi-tasking model including the same according to an embodiment of the present disclosure.

FIG. 24 is a schematic example of a conceptual diagram for describing a method for sampling data for a general-purpose multi-tasking model and a method for providing a general-purpose multi-tasking model including the same according to an embodiment of the present disclosure.

FIG. 25 is a schematic block flowchart for describing a method for providing a guide supporting performance enhancement of a multi-tasking model according to an embodiment of the present disclosure.

FIG. 26 is a schematic example of a diagram for describing a validity index according to an embodiment of the present disclosure.

FIG. 27 is a schematic example of a diagram for describing a background of utilization of a density estimation algorithm according to an embodiment of the present disclosure.

FIG. 28 are schematic examples of output data based on a density estimation algorithm according to an embodiment of the present disclosure.

FIG. 29 is a schematic example of visualization of a physical property guide according to an embodiment of the present disclosure.

FIG. 30 is a schematic example for describing a physical property change interface according to an embodiment of the present disclosure.

### DETAILED DESCRIPTION

In the following description, for the purposes of explanation, numerous specific details are set forth in order to provide a thorough understanding of various embodiments or implementations of the invention. As used herein “embodiments” and “implementations” are interchangeable words that are non-limiting examples of devices or methods employing one or more of the inventive concepts disclosed herein. It is apparent, however, that various embodiments may be practiced without these specific details or with one or more equivalent arrangements. In other instances, well-known structures and devices are shown in block diagram form in order to avoid unnecessarily obscuring various embodiments. Further, various embodiments may be different, but do not have to be exclusive. For example, specific shapes, configurations, and characteristics of an embodiment may be used or implemented in another embodiment without departing from the inventive concepts.

Unless otherwise specified, the illustrated embodiments are to be understood as providing features of varying detail of some ways in which the inventive concepts may be implemented in practice. Therefore, unless otherwise specified, the features, components, modules, layers, films, panels, regions, and/or aspects, etc. (hereinafter individually or collectively referred to as “elements”), of the various embodiments may be otherwise combined, separated, interchanged, and/or rearranged without departing from the inventive concepts.

The use of cross-hatching and/or shading in the accompanying drawings is generally provided to clarify boundaries between adjacent elements. As such, neither the presence nor the absence of cross-hatching or shading conveys or indicates any preference or requirement for particular materials, material properties, dimensions, proportions, commonalities between illustrated elements, and/or any other characteristic, attribute, property, etc., of the elements, unless specified. Further, in the accompanying drawings, the size and relative sizes of elements may be exaggerated for clarity and/or descriptive purposes. When an embodiment may be implemented differently, a specific process order may be performed differently from the described order. For example, two consecutively described processes may be performed substantially at the same time or performed in an order opposite to the described order. Also, like reference numerals denote like elements.

When an element, such as a layer, is referred to as being “on,” “connected to,” or “coupled to” another element or layer, it may be directly on, connected to, or coupled to the other element or layer or intervening elements or layers may be present. When, however, an element or layer is referred to as being “directly on,” “directly connected to,” or “directly coupled to” another element or layer, there are no intervening elements or layers present. To this end, the term “connected” may refer to physical, electrical, and/or fluid connection, with or without intervening elements. Further, the D1-axis, the D2-axis, and the D3-axis are not limited to three axes of a rectangular coordinate system, such as the x, y, and z-axes, and may be interpreted in a broader sense. For example, the D1-axis, the D2-axis, and the D3-axis may be perpendicular to one another, or may represent different directions that are not perpendicular to one another. For the purposes of this disclosure, “at least one of X, Y, and Z” and “at least one selected from the group consisting of X, Y, and Z” may be construed as X only, Y only, Z only, or any combination of two or more of X, Y, and Z, such as, for instance, XYZ, XYY, YZ, and ZZ. As used herein, the term “and/or” includes any and all combinations of one or more of the associated listed items.

Although the terms “first,” “second,” etc. may be used herein to describe various types of elements, these elements should not be limited by these terms. These terms are used to distinguish one element from another element. Thus, a first element discussed below could be termed a second element without departing from the teachings of the disclosure.

Spatially relative terms, such as “beneath,” “below,” “under,” “lower,” “above,” “upper,” “over,” “higher,” “side” (e.g., as in “sidewall”), and the like, may be used herein for descriptive purposes, and, thereby, to describe one elements relationship to another element(s) as illustrated in the drawings. Spatially relative terms are intended to encompass different orientations of an apparatus in use, operation, and/or manufacture in addition to the orientation depicted in the drawings. For example, if the apparatus in the drawings is turned over, elements described as “below” or “beneath” other elements or features would then be oriented “above” the other elements or features. Thus, the exemplary term “below” can encompass both an orientation of above and below. Furthermore, the apparatus may be otherwise oriented (e.g., rotated 90 degrees or at other orientations), and, as such, the spatially relative descriptors used herein interpreted accordingly.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof. It is also noted that, as used herein, the terms “substantially,” “about,” and other similar terms, are used as terms of approximation and not as terms of degree, and, as such, are utilized to account for inherent deviations in measured, calculated, and/or provided values that would be recognized by one of ordinary skill in the art.

Various embodiments are described herein with reference to sectional and/or exploded illustrations that are schematic illustrations of idealized embodiments and/or intermediate structures. As such, variations from the shapes of the illustrations as a result, for example, of manufacturing techniques and/or tolerances, are to be expected. Thus, embodiments disclosed herein should not necessarily be construed as limited to the particular illustrated shapes of regions, but are to include deviations in shapes that result from, for instance, manufacturing. In this manner, regions illustrated in the drawings may be schematic in nature and the shapes of these regions may not reflect actual shapes of regions of a device and, as such, are not necessarily intended to be limiting.

As customary in the field, some embodiments are described and illustrated in the accompanying drawings in terms of functional blocks, units, and/or modules. Those skilled in the art will appreciate that these blocks, units, and/or modules are physically implemented by electronic (or optical) circuits, such as logic circuits, discrete components, microprocessors, hard-wired circuits, memory elements, wiring connections, and the like, which may be formed using semiconductor-based fabrication techniques or other manufacturing technologies. In the case of the blocks, units, and/or modules being implemented by microprocessors or other similar hardware, they may be programmed and controlled using software (e.g., microcode) to perform various functions discussed herein and may optionally be driven by firmware and/or software. It is also contemplated that each block, unit, and/or module may be implemented by dedicated hardware, or as a combination of dedicated hardware to perform some functions and a processor (e.g., one or more programmed microprocessors and associated circuitry) to perform other functions. Also, each block, unit, and/or module of some embodiments may be physically separated into two or more interacting and discrete blocks, units, and/or modules without departing from the scope of the inventive concepts. Further, the blocks, units, and/or modules of some embodiments may be physically combined into more complex blocks, units, and/or modules without departing from the scope of the inventive concepts.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and should not be interpreted in an idealized or overly formal sense, unless expressly so defined herein.

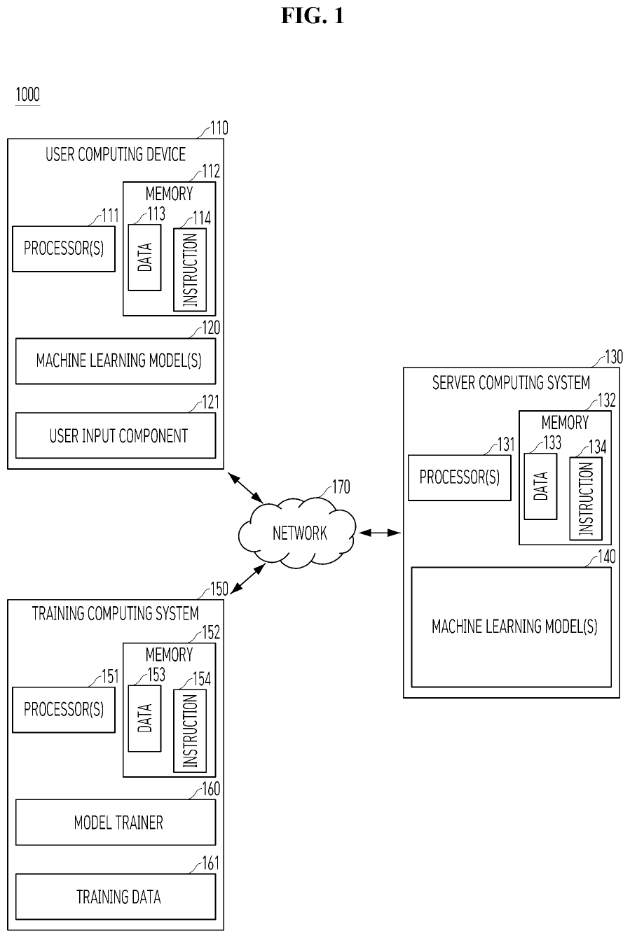

FIG. 1 illustrates an example of a block diagram of a computing system according to an embodiment of the present disclosure.

Referring to FIG. 1, a computing system 1000 according to an embodiment of the present disclosure includes a user computing device 110, a server computing system 130, and a training computing system 150, and the devices are communicable through a network 170.

A method and a system for providing a guide supporting performance enhancement of a multi-tasking model, a method for sampling data for a general-purpose multi-tasking model, and a method and a system for providing a general-purpose multi-tasking model including the same, according to an embodiment of the present disclosure, may be 1) implemented and provided locally by the user computing device 110, 2) implemented and provided in the form of a web service by the server computing system 130 in communication with the user computing device 110, and 3) implemented and provided by the user computing device 110 and the server computing system 130 in association with each other.

In this regard, in an embodiment, the user computing device 110 and/or the server computing system 130 may train machine learning models 120 and/or 140 through interaction with the training computing system 150 communicatively connected thereto via the network 170. The training computing system 150 may be separate from the server computing system 130. In another embodiment, the training computing system 150 may be part of the server computing system 130.

In this regard, an artificial intelligence model may be 1) directly trained by the user computing device 110 locally, 2) trained by the server computing system 130 and the user computing device 110 interacting with each other through the network 170, and 3) trained by the separate training computing system 150 using various training techniques and learning techniques. Further, the artificial intelligence model trained by the training computing system 150 may be transmitted to the user computing device 110 and/or the server computing system 130 through the network 170 for provision/update.

In some embodiments, the training computing system 150 may be part of the server computing system 130, or may be part of the user computing device 110.

The user computing device 110 may include all other types of computing devices such as smart phones, mobile phones, digital broadcasting devices, personal digital assistants (PDA), portable multimedia players (PMP), desktops, wearable devices, embedded computing devices, and/or tablet PCs.

Such a user computing device 110 includes at least one processor 111 and a memory 112. The processor 111 may include at least one of a central processing unit (CPU), a graphics processing unit (GPU), an application specific integrated circuit (ASIC), a digital signal processor (DSP), a digital signal processing device (DSPD), a programmable logic device (PLD), a field programmable gate array (FPGA), a controller, a micro-controller, a microprocessor, and/or electrical units for executing other functions, or a plurality of electrically connected processors.

The memory 112 may include one or more non-transitory/transitory computer-readable storage media such as RAM, ROM, EEPROM, EPROM, flash memory devices, magnetic disks, and the like, and combinations thereof, and may include web storage of a server that executes a storage function of the memory on the Internet. Such a memory 112 may store data 113 and instructions 114 necessary for the at least one processor 111 to execute functional operations such as training an artificial intelligence model or executing multi-tasking learning through the artificial intelligence model.

In an embodiment, the user computing device 110 may store one or more machine learning models 120.

In detail, the machine learning models 120 may be various machine learning models, such as a plurality of neural networks (e.g., a deep neural network), or other types of machine learning models, including non-linear models and/or linear models, or may be composed of combinations thereof.

The neural networks may include at least one of a feed-forward neural network, a recurrent neural network (e.g., long and short-term memory recurrent neural networks), a convolutional neural network, and/or other types of neural networks.

In an embodiment, the user computing device 110 may receive one or more machine learning models 120 from the server computing system 130 through the network 170, store the machine learning models 120 in the memory 112, and then execute the stored machine learning models 120 by the processor 111 to execute multi-tasking learning and the like.

In another embodiment, the server computing system 130 may include one or more machine learning models 140 to execute an operation using the machine learning models 140, and may provide a multi-tasking model guide provision service and a general-purpose multi-tasking model provision service to a user by interoperating with the user computing device 110 in a manner of exchanging data related thereto with the user computing device 110.

For example, the user computing device 110 may execute the multi-tasking model guide provision service and the general-purpose multi-tasking model provision service in a manner in which the server computing system 130 provides output for user's input using the machine learning models 140 through the web.

In addition, the artificial intelligence models may be implemented such that at least a portion of the machine learning models 120 and/or 140 is executed on the user computing device 110, while the remaining portion is executed on the server computing system 130.

In addition, the user computing device 110 may include at least one input component 121 that senses a user's input. For example, the user input component 121 may include a touch sensor (e.g., a touch screen and/or a touch pad) that senses a touch of a user's input medium (e.g., a finger or a stylus), an image sensor that senses a user's motion input, a microphone that senses a user's voice input, a button, a mouse, and/or a keyboard. In addition, when receiving input to an external controller (e.g., a mouse and/or a keyboard) through an interface, the user input component 121 may include the interface and the external controller.

The server computing system 130 includes at least one processor 131 and a memory 132. The processor 131 may include at least one of a central processing unit (CPU), a graphics processing unit (GPU), an application specific integrated circuit (ASIC), a digital signal processor (DSP), a digital signal processing device (DSPD), a programmable logic device (PLD), a field programmable gate array (FPGA), a controller, a micro-controller, a microprocessor, and/or electrical units for executing other functions, or a plurality of electrically connected processors.

In addition, the memory 132 may include one or more non-transitory/transitory computer-readable storage media such as RAM, ROM, EEPROM, EPROM, flash memory devices, magnetic disks, and the like, and combinations thereof. Such a memory 132 may store data 133 and instructions 134 necessary for the processor 131 to execute functional operations such as training an artificial intelligence model or executing multi-tasking learning through the artificial intelligence model.

In an embodiment, the server computing system 130 may be implemented including at least one computing device. For example, the server computing system 130 may be implemented to operate a plurality of computing devices according to a sequential computing architecture, a parallel computing architecture, or a combination thereof. In addition, the server computing system 130 may include a plurality of computing devices connected to each other via the network 170.

In addition, the server computing system 130 may store the one or more machine learning models 140. For example, the server computing system 130 may include neural networks and/or other multi-layer non-linear models as the machine learning models 140. Exemplary neural networks may include feed forward neural networks, deep neural networks, recurrent neural networks, and convolutional neural networks.

The training computing system 150 includes at least one processor 151 and a memory 152. The processor 151 may include at least one of a central processing unit (CPU), a graphics processing unit (GPU), an application specific integrated circuit (ASIC), a digital signal processor (DSP), a digital signal processing device (DSPD), a programmable logic device (PLD), a field programmable gate array (FPGA), a controller, a micro-controller, a microprocessor, and/or electrical units for executing other functions, or a plurality of electrically connected processors.

In addition, the memory 152 may include one or more non-transitory/transitory computer-readable storage media such as RAM, ROM, EEPROM, EPROM, flash memory devices, magnetic disks, and the like, and combinations thereof. Such a memory 152 may store data 153 and instructions 154 necessary for the processor 151 to execute training of an artificial intelligence model or the like.

For example, the training computing system 150 may include a model trainer 160 that trains the machine learning models 120 and/or 140 stored in the user computing device 110 and/or the server computing system 130 using various training or learning techniques such as backpropagation of errors (according to the framework shown in FIG. 3).

As an example, such a model trainer 160 may execute updates of one or more parameters of the machine learning models 120 and/or 140 in a backpropagation manner based on a defined loss function.

In some implementations, executing the backpropagation of errors may include executing truncated backpropagation through time. The model trainer 160 may execute a number of generalization techniques (e.g., weight reduction, drop-out, knowledge distillation, etc.) to enhance the generalization ability of the trained machine learning models 120 and/or 140.

In particular, the model trainer 160 may train the machine learning models 120 and/or 140 based on a series of training data 161. The training data 161 may include various modalities, such as images, audio samples, and/or text. Examples of image data that are used may include video frames, LiDAR point clouds, X-ray images, computed tomography scans, hyperspectral images, and/or various other forms of images.

Such training data 161 may be provided by the user computing device 110 and/or the server computing system 130. When the training computing device trains the machine learning models 120 and/or 140 on specific data of the user computing device 110, the machine learning models 120 and/or 140 may be characterized as personalized models.

In addition, the model trainer 160 includes computer logic utilized to provide desired functionality.

In addition, the model trainer 160 may be implemented as hardware, firmware, and/or software that controls a general-purpose processor. In one implementation, the model trainer 160 may include at least one program file stored in a storage device, loaded into the memory 152, and executed by the one or more processors 151. In another implementation, the model trainer 160 may include one or more sets of computer-executable data 153 and instructions 154 stored in tangible computer-readable storage media, such as RAM hard disks or optical or magnetic media.

The network 170 includes, but is not limited to, a 3rd generation partnership project (3GPP) network, a long term evolution (LTE) network, a world interoperability for microwave access (WIMAX) network, the Internet, a local area network (LAN), a wireless local area network (wireless LAN), a wide area network (WAN), a personal area network (PAN), a Bluetooth network, a satellite broadcasting network, an analog broadcasting network, and/or a digital multimedia broadcasting (DMB) network.

In general, communication over the network 170 may be executed using any type of wired and/or wireless connection, via various communication protocols (e.g., TCP/IP, HTTP, SMTP, and/or FTP), encoding or formats (e.g., HTML and/or XML), and/or protection schemas (e.g., VPN, secure HTTP, and/or SSL).

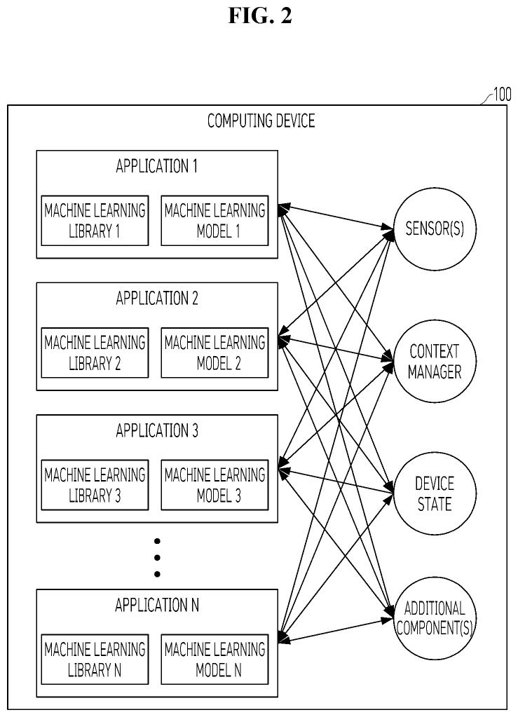

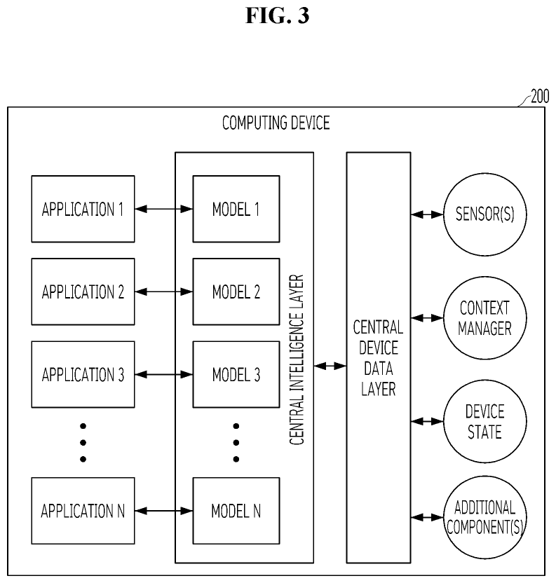

FIG. 2 illustrates an example of a block diagram of a computing device according to an embodiment of the present disclosure.

Referring to FIG. 2, a computing device 100 included in each of the user computing device 110, the server computing system 130, and the training computing system 150 includes multiple applications (e.g., applications 1 to N). Each application may include a machine learning library and one or more machine learning models. For example, the applications may include an image processing (e.g., detection, classification, and/or segmentation) application, a text messaging application, an email application, a dictation application, a virtual keyboard application, a browser application, and/or a chat-bot application.

In an embodiment, the computing device 100 may include the model trainer 160 for training artificial intelligence models, and may store and operate the trained artificial intelligence models, thereby providing output data according to predetermined input data (e.g., intrinsic material characteristic information and/or material physical property-specific information).

Each application of the computing device 100 may be in communication with a number of other components of the computing device 100, such as, for example, at least one sensor, a context manager, a device status component, and/or additional components. In an embodiment, each application may be in communication with each device component using an API (e.g., a public API). In an embodiment, an API used by each application may be specific to that application.

FIG. 3 illustrates an example of a block diagram from another perspective of a computing device according to an embodiment of the present disclosure.

Referring to FIG. 3, a computing device 200 includes multiple applications (e.g., applications 1 to N). Each application may be in communication with a central intelligence layer. For example, the applications may include an image processing application, a text message application, an email application, a dictation application, a virtual keyboard application, and/or a browser application. In an embodiment, each application may be in communication with the central intelligence layer (and models stored therein) using an API (e.g., a common API across all applications).

The central intelligence layer may include multiple machine learning models. For example, as shown in FIG. 3, at least some of the machine learning models may be provided for the respective applications and managed by the central intelligence layer. In another implementation, two or more applications may share a single machine learning model. For example, in some implementations, the central intelligence layer may provide a single model for all applications. In some implementations, the central intelligence layer may be included within the operating system of the computing device 200 or implemented otherwise.

The central intelligence layer may be in communication with a central device data layer. The central device data layer may be centralized data storage for the computing device 200. As shown in FIG. 3, the central device data layer may be in communication with multiple other components of the computing device 200, such as, for example, one or more sensors, a context manager, a device status component, and/or additional components. In some implementations, the central device data layer may be in communication with each device component using an API (e.g., a private API).

The technology described herein may refer to servers, databases, software applications, and other computer-based systems, as well as actions taken and information transmitted to or from such systems. It will be appreciated that the inherent flexibility of computer-based systems allows for a wide range of possible configurations, combinations, divisions of tasks, and functionality among components and from such components. For example, the processes described herein may be implemented using a single device or component, or multiple devices or components operating in combination. Databases and applications may be implemented in a single system or in distributed systems across multiple systems. Distributed components may operate sequentially or in parallel.

### [Multi-Tasking Learning Model MtLM]

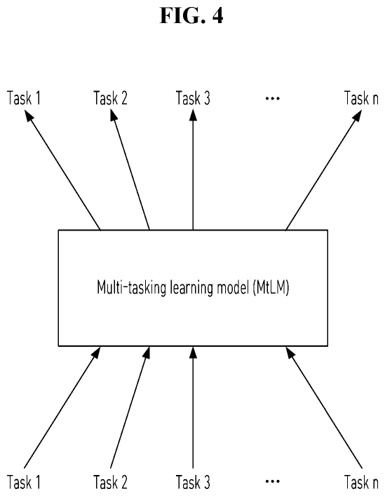

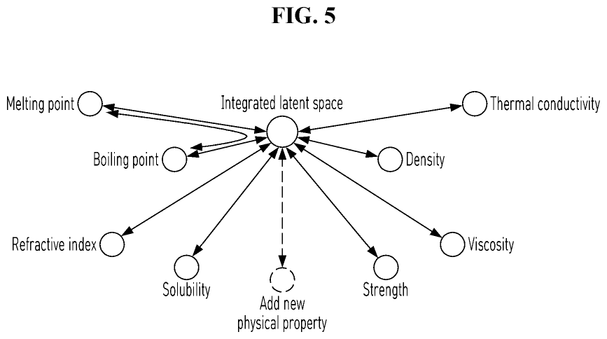

FIGS. 4 and 5 illustrate examples of conceptual diagrams for describing a multi-tasking learning model MtLM according to an embodiment of the present disclosure.

Referring to FIGS. 4 and 5, a multi-tasking learning model MtLM (geometrically aligned transfer encoder model) according to an embodiment of the present disclosure may be a machine learning model in which knowledge data (e.g., latent vectors, etc.) fragmented in latent spaces for respective tasks are mutually aligned in a single integrated latent space M (manifold) through a geometric transfer in order to process multi-tasks for an integrated output satisfying a plurality of domains.

For reference, a latent space according to an embodiment may refer to a virtual space in which predetermined data is located after being converted through an encoding process. In this space, important characteristics of the data may be represented in a compressed form.

The integrated latent space M, according to an embodiment, may refer to a space in which data originating from different latent spaces are geometrically represented within a single integrated virtual space. A transformation between one individual latent space and the integrated latent space M may serve to align geometric characteristics of the data.

In other words, the multi-tasking learning model MtLM, according to an embodiment, may not only learn knowledge data corresponding to various domains simultaneously but also efficiently learn relationships among various domains Accordingly, the MtLM may execute effective multi-tasking learning that expands a learning area while simultaneously achieving collective learning of a local pattern corresponding to each domain and a common principle shared across the plurality of domains.

Accordingly, the multi-tasking learning model MtLM may directly enhance the processing performance and accuracy of various multi-tasking tasks based on the model trained as described above.

In the following embodiment, the multi-tasking learning model MtLM will be described using an example involving relationships between a predetermined material and a plurality of physical properties, and will be explained as a learning model that performs multiple tasking, including a first task of predicting characteristics of a first physical property of the material and a second task of predicting characteristics of a second physical property of the material, among others. However, it should be understood that the present disclosure is not limited to a learning method and a prediction method for multi-tasking relationships between a material and a plurality of physical properties, and may be applied to all various tasks in which a plurality of tasks, such as relationships between a material and a plurality of physical properties, are to be simultaneously executed.

Accordingly, a domain according to the following embodiment may refer to a category (field) for predicting a predetermined physical property (e.g., boiling point, melting point, refractive index, solubility, viscosity, surface tension, density, strength, and/or thermal conductivity).

In an embodiment, such a multi-tasking learning model MtLM may execute pre-learning based on predetermined experimental data.

The experimental data according to an embodiment may refer to training data used for training the multi-tasking learning model MtLM, and may include predetermined input data and output data (i.e., label) information corresponding thereto.

In an embodiment, such experimental data may include predetermined intrinsic material characteristic information and material physical property-specific information corresponding thereto. In particular, in an embodiment, the experimental data may be configured such that predetermined intrinsic material characteristic information is used as input data, and material physical property-specific information is included as output data (i.e., label) mapped to the input intrinsic material characteristic information.

The intrinsic material characteristic information according to an embodiment may be information specifying unique characteristics possessed by a predetermined material.

For example, the intrinsic material characteristic information may include at least one of a predetermined material name, a molecular structural formula, and/or chemical formula data. In the following description, the intrinsic material characteristic information is limited to molecular structural formula (e.g., n-dimensional (n>=2) molecular structural formula) data.

In addition, the material physical property-specific information according to an embodiment may be information specifying data values (i.e., characteristic values of physical properties, where the values include ranges) that a predetermined material has with respect to predetermined physical properties.

For example, the material physical property-specific information may include physical property (i.e., domain) values such as boiling point, melting point, refractive index, solubility, viscosity, surface tension, density, strength, and/or thermal conductivity of a predetermined material.

In an embodiment, the multi-tasking learning model MtLM may use molecular structural formula data indicating the intrinsic material characteristic information as input data, and may use physical property value data for each task as output data.

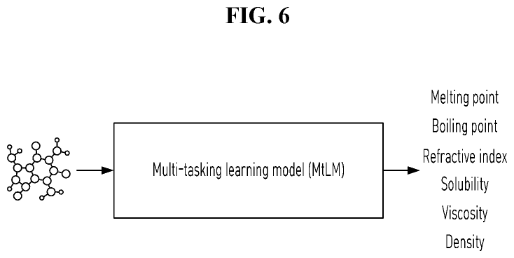

FIG. 6 illustrates an example of a conceptual diagram of a multi-tasking learning model for predicting a plurality of physical property values for a specific material according to an embodiment of the present disclosure.

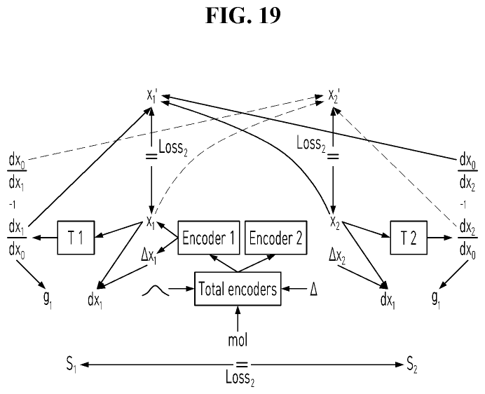

Therefore, referring to FIG. 19, the multi-tasking learning model MtLM according to an embodiment of the present disclosure may be a multi-tasking model that executes multi-tasks of predicting outputs for the plurality of respective domains (e.g., a plurality of physical properties such as boiling point, melting point, refractive index, solubility, viscosity, surface tension, density, strength, and/or thermal conductivity) based on predetermined input data (e.g., predetermined molecular structural formula data, etc.).

Accordingly, the above-described tasks may be to predict outputs (i.e., the plurality of physical property values) for the plurality of respective domains with respect to input data (e.g., the predetermined molecular structural formula data, etc.).

In an embodiment, such tasks may include a first task for predicting a first physical property value of a predetermined material, a second task for predicting a second physical property value of the material, . . . , and an n-th task for predicting an n-th (n>=2) physical property value of the material, and each of the tasks described above may include a plurality of sub-tasks for predicting a physical property value mapped to that task.

In particular, in an embodiment, the tasks may include a plurality of sub-tasks for predicting the physical property value mapped to the n-th task, and a main-task including the plurality of sub-tasks. The main task may correspond to the n-th task mapped thereto.

In addition, the tasks according to an embodiment may include a source task that is a task of an entity providing data transferred in the transfer learning process according to an embodiment of the present disclosure, and a target task that is a task of an entity receiving the transferred data.

In other words, in an embodiment, a task may be defined as a source task or a target task depending on whether the task corresponds to an entity that transfers data during a learning process or an entity that receives transferred data.

In other words, the multi-tasking learning model MtLM, having undergone pre-learning according to an embodiment of the present disclosure, may receive predetermined intrinsic material characteristic information and/or material physical property-specific information and may output predicted data based on the input information and the knowledge acquired through training.

In an embodiment, the multi-tasking learning model MtLM may receive predetermined intrinsic material characteristic information and output material physical property-specific information predicted based on the input information and learned knowledge.

In this regard, according to embodiments, the multi-tasking learning model MtLM may receive predetermined material physical property-specific information, and output intrinsic material characteristic information predicted based on the input information and learned knowledge.

In particular, the multi-tasking learning model MtLM may include a model that is reverse-designed to predict and output intrinsic material characteristic information based on predetermined material physical property-specific information.

In addition, according to embodiments, the multi-tasking learning model MtLM may receive predetermined intrinsic material characteristic information and material physical property-specific information, and output optimal intrinsic material characteristic information and material physical property-specific information predicted based on the input information and learned knowledge.

In particular, the multi-tasking learning model MtLM may include a model that is reverse-designed to output optimal intrinsic material characteristic information and material physical property-specific information predicted based on predetermined intrinsic material characteristic information and material physical property-specific information.

As described above, in the multi-tasking learning model MtLM according to an embodiment of the present disclosure, during the process of simultaneously learning tasks for predicting a plurality of physical properties of a predetermined material, the model may learn not only principles governing relationships between the material and individual physical properties but also interrelationships among the physical properties and a common principle shared across all of the learned physical properties. Accordingly, the MtLM may enable more accurate prediction of the physical property values and facilitate subsequent updates.

In addition, because training data on a plurality of physical properties are related to various materials, the multi-tasking learning model MtLM may execute learning on materials in a more expanded range, thereby also further expanding a material range predictable for each physical property.

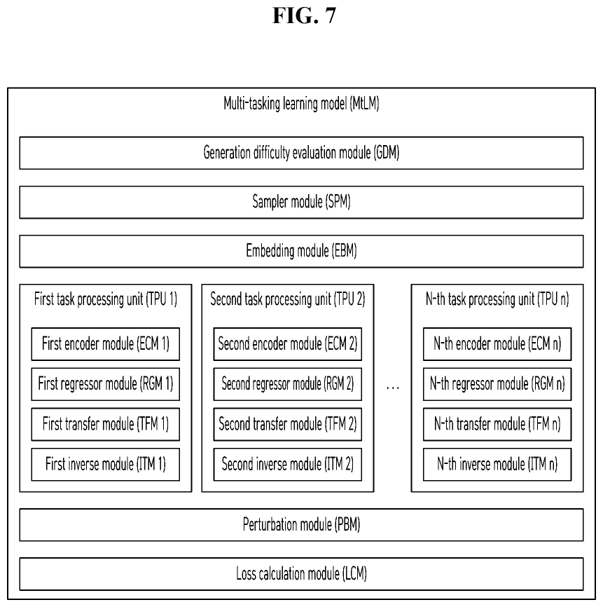

FIG. 7 illustrates an internal block diagram of a multi-tasking learning model MtLM according to an embodiment of the present disclosure.

Referring to FIG. 7, in another aspect, a multi-tasking learning model MtLM according to an embodiment may include at least one embedding module EBM, at least one task processing unit TPU, at least one encoder module ECM, at least one regressor module RGM, at least one transfer module TFM, at least one inverse transfer module ITM, at least one perturbation module PBM, at least one loss calculation module LCM, at least one sampler module SPM, and at least one generation difficulty evaluation module GDM.

In detail, the embedding module EBM according to an embodiment of the present disclosure may be a pre-encoder module that converts predetermined input data into embedding vectors.

Specifically, the embedding module EBM may compress molecular structural formula data, which constitutes high-dimensional data, into embedding vectors, which serve as low-dimensional representations. This dimensionality reduction may decrease the size of inputs processed by an encoder, thereby improving computational efficiency and learning speed, and may enable the encoder to be trained with a focus on salient features of the molecular structural formula for pre-learning tasks.

Accordingly, useful features from a model trained on a source task may be easily applied to a model to be trained on a target task, and overlapping features among different domains may be generalized, thereby effectively executing transfer learning for new domains/tasks.

In particular, the embedding module EBM may be a module that projects specific input data into a predetermined embedding space and converts the input data into a vector format.

In an embodiment, as the embedding module EBM, a graph neural network (GNN) suitable for molecular structural formula feature extraction may be used, and for example, embedding vectors for input data may be provided based on a directed message passing neural network (DMPNN) structure.

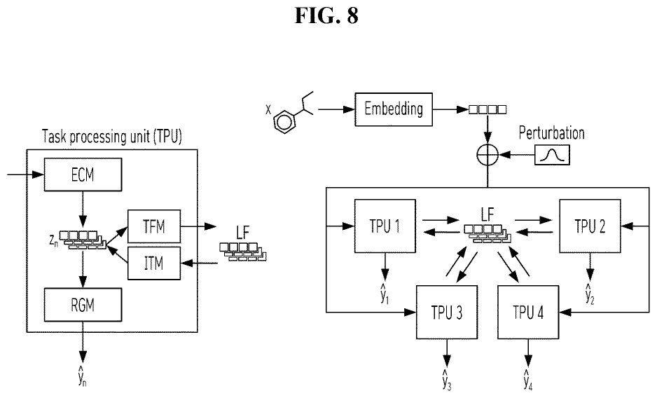

FIG. 8 illustrates an example of a conceptual diagram for describing a multi-tasking learning model MtLM including a plurality of task processing units TPU according to an embodiment of the present disclosure.

In addition, referring to FIG. 8, a task processing unit TPU according to an embodiment of the present disclosure may be a module that executes learning and prediction processes based on predetermined tasks.

In an embodiment, such task processing units TPU may include a first task processing unit corresponding to a first domain (e.g., boiling point), a second task processing unit corresponding to a second domain (e.g., melting point), . . . , and an n-th task processing unit corresponding to an n-th domain.

In particular, in an embodiment, the task processing units TPU may include a plurality of first to n-th task processing units TPU corresponding to the number of given domains (i.e., physical properties).

In this regard, in an embodiment, one task processing unit TPU among the plurality of task processing units TPU may be a source task processing unit that is a task processing unit TPU corresponding to a source task of transfer learning according to an embodiment of the present disclosure.

In addition, one task processing unit TPU among the remaining task processing units TPU excluding the source task processing unit may be a target task processing unit that is a task processing unit TPU corresponding to a target task of transfer learning according to an embodiment of the present disclosure.

In detail, the task processing unit TPU according to an embodiment may include at least one encoder module ECM, at least one regressor module RGM, at least one transfer module TFM, and at least one inverse module ITM.

Specifically, the encoder module ECM according to an embodiment of the present disclosure may be a module that receives predetermined embedding vectors as input, projects the input embedding vectors into a latent space corresponding to a corresponding task, and converts the same into latent vectors.

In particular, the encoder module ECM may extract main features of the input embedding vectors and represent those features within a corresponding latent space. More specifically, the encoder module ECM may identify important features among features of the embedding vectors, and may execute data compression by progressively reducing the dimensionality of the data while removing unnecessary information or noise. As a result, the encoder module (ECM) may output latent vectors that serve as representations in the latent space.

In an embodiment, such encoder modules ECM may include a plurality of encoder modules ECM respectively corresponding to a plurality of domains.

In an embodiment, the encoder modules ECM may include a first encoder module corresponding to a first domain (e.g., boiling point), a second encoder module corresponding to a second domain (e.g., melting point), and an n-th encoder module corresponding to an n-th domain.

In another embodiment, the encoder modules ECM may include a third encoder module for executing a first task of predicting solubility in a first solvent corresponding to a third domain (e.g., solubility), and a fourth encoder module for executing a second task of predicting solubility in a second solvent. In particular, in another embodiment, multi-tasking may be executed for different tasks for within the same domain. In one example, a multi-tasking model that integrates multi-tasking across different domains and multi-tasking for different tasks within the same domain may also be included as an embodiment of the present disclosure.

Hereinafter, the description will be made based on an assumption that different domains correspond to different tasks.

In this regard, in an embodiment, one encoder module ECM among the plurality of encoder modules ECM may be a source encoder module that is an encoder module ECM corresponding to a source task of transfer learning according to an embodiment of the present disclosure.

In addition, one encoder module ECM among the remaining encoder modules ECM excluding the source encoder module may be a target encoder module that is an encoder module ECM corresponding to a target task of transfer learning according to an embodiment of the present disclosure.

In addition, the regressor module RGM according to an embodiment of the present disclosure may be a head module that receives predetermined latent vectors as input and generates a final prediction value according to the input latent vectors. In particular, in an embodiment, the regressor module RGM is used as an example of the head module.

Such a regressor module RGM may be directly involved in generating the final output to determine the prediction performance of the model.

In addition, in an embodiment, the regressor modules RGM may include a plurality of regressor modules RGM respectively corresponding to a plurality of domains.

In an embodiment, the regressor modules RGM may include a first regressor module corresponding to a first domain (e.g., boiling point), a second regressor module corresponding to a second domain (e.g., melting point), . . . , and an n-th regressor module corresponding to an n-th domain.

In this regard, in an embodiment, one regressor module RGM among the plurality of regressor modules RGM may be a source regressor module that is a regressor module RGM corresponding to a source task of transfer learning according to an embodiment of the present disclosure.

In addition, one regressor module RGM among the remaining regressor modules RGM excluding the source regressor module may be a target regressor module that is a regressor module RGM corresponding to a target task of transfer learning according to an embodiment of the present disclosure.

In addition, the transfer module TFM according to an embodiment of the present disclosure may be a module that maps predetermined latent vectors to a latent space of another task and converts the mapped latent vectors into transfer vectors.

In detail, in an embodiment, the transfer module TFM may map specific latent vectors to a latent space of another task through the integrated latent space M based on Riemannian geometry and convert the mapped latent vectors into transfer vectors.

In this process, the transfer module TFM may implement geometric alignment among the mapped tasks according to an embodiment of the present disclosure. This will be described later in detail in the following multi-tasking model training method.

In particular, in an embodiment, the transfer module TFM may effectively execute a transfer of knowledge data among a plurality of tasks by mapping latent vectors according to a first task to a latent space according to a second task through geometric alignment according to an embodiment of the present disclosure.

In this regard, in an embodiment, the transfer module TFM may support data processing that enhances the accuracy and consistency of the converted vector (i.e., the transfer vector) by utilizing an autoencoder structure.

In addition, in an embodiment, the transfer modules TFM may include a plurality of transfer modules TFM respectively corresponding to a plurality of domains.

In an embodiment, the transfer modules TFM may include a first transfer module corresponding to a first domain (e.g., boiling point), a second transfer module corresponding to a second domain (e.g., melting point), . . . , and an n-th transfer module corresponding to an n-th domain.

In this regard, in an embodiment, one transfer module TFM among the plurality of transfer modules TFM may be a source transfer module that is a transfer module TFM corresponding to a source task of transfer learning according to an embodiment of the present disclosure.

In addition, one transfer module TFM among the remaining transfer modules TFM excluding the source transfer module may be a target transfer module that is a transfer module TFM corresponding to a target task of transfer learning according to an embodiment of the present disclosure.

In addition, the inverse transfer module ITM according to an embodiment of the present disclosure may be a module that reconstructs the transfer vectors mapped and converted into the latent space of another task by the transfer module TFM so as to be mapped back to the original latent space.

Thus, in an embodiment, the inverse module ITM may generate vectors (hereinafter, inverse vectors) obtained by reconstructing and converting the transfer vectors back to its original state.

In this regard, in an embodiment, the inverse module ITM may enhance the stability of the above-described reconstruction process and the accuracy and consistency of the corresponding transfer vectors by utilizing the autoencoder structure.

In an embodiment, such inverse modules ITM may include a plurality of inverse modules ITM respectively corresponding to a plurality of domains.

In an embodiment, the inverse modules ITM may include a first inverse module corresponding to a first domain (e.g., boiling point), a second inverse module corresponding to a second domain (e.g., melting point), . . . , and an n-th inverse module corresponding to an n-th domain.

In this regard, in an embodiment, one inverse module ITM among the plurality of inverse modules ITM may be a source inverse module that is an inverse module ITM corresponding to a source task of transfer learning according to an embodiment of the present disclosure.

In addition, one inverse module ITM among the remaining inverse modules ITM excluding the source inverse module may be a target inverse module that is an inverse module ITM corresponding to a target task of transfer learning according to an embodiment of the present disclosure.

As such, in an embodiment, the multi-tasking learning model MtLM includes the plurality of task processing units TPU respectively corresponding to the plurality of domains (i.e., physical properties), thereby defining latent spaces for the respective plurality of tasks executed based thereon.

In addition, the multi-tasking learning model MtLM may simultaneously learn a transformation in which the plurality of defined latent spaces are transformed into one integrated latent space M according to a pre-training method to be described later.

In other words, the multi-tasking learning model MtLM may execute learning for geometrically aligning n latent spaces according to various physical properties into one integrated latent space M according to the pre-training method to be described below through the plurality of task processing units TPU.

Therefore, the multi-tasking learning model MtLM may implement simultaneous/collective transfer learning for n*n physical property combinations when considering n physical properties.

Therefore, the multi-tasking learning model MtLM according to an embodiment may enhance the prediction performance according to information sharing and learning among interrelated physical properties/tasks.

In an embodiment, the multi-tasking learning model MtLM may implement the transformation between the plurality of latent spaces and the integrated latent space M in the same manner regardless of configurations of physical property combinations (e.g., the first task-second task combination or the second task-third task combination).

The multi-tasking learning model MtLM may execute the transformation between latent spaces of the multi-tasks corresponding to various physical properties and the integrated latent space M in the same manner.

Accordingly, the multi-tasking learning model MtLM may execute data processing that aligns geometric characteristics based on respective latent vectors of a plurality of latent spaces onto the integrated latent space M in a consistent manner.

Therefore, the multi-tasking learning model MtLM may effectively maintain the consistency of the transformation from each latent space to the integrated latent space M. Thus, the multi-tasking learning model MtLM may more stably support flow of information among tasks.

For example, when the transformation from the latent space of the first task to the integrated latent space M and the transformation from the latent space of the second task to the integrated latent space M are the same as each other, the multi-tasking learning model MtLM may usefully utilize information obtained from the first task for the second task.

Therefore, the multi-tasking learning model MtLM may enhance the sharing of knowledge data among multi-tasks, and simultaneously, may further enhance the prediction performance and stability of the model.

Referring back to FIG. 7, a perturbation module PBM according to an embodiment of the present disclosure may be a module that generates a plurality of perturbation vectors by applying predetermined changes to predetermined embedding vectors.

In detail, in an embodiment, the perturbation module PBM may be a module that applies a change of moving specific embedding vectors in a predetermined direction, thereby generating a plurality of perturbation vectors (i.e., perturbation points) on a periphery of the corresponding embedding vectors.

The plurality of generated perturbation vectors may be designed to maintain relative distances to the corresponding embedding vectors, thereby effectively assisting geometric alignment.

In particular, the above-described perturbation module PBM may generate a plurality of perturbation vectors to assist geometric alignment of the model, thereby helping to align coordinate systems between a source task and a target task.

In addition, in an embodiment, the perturbation module PBM may calculate distances between predetermined embedding vectors and a plurality of perturbation vectors generated based thereon, and may support alignment of displacements between a source task and a target task based on the calculated distances.

Accordingly, the perturbation module PBM may more easily maintain consistency in the latent space for the model.

According to an embodiment, the perturbation module PBM may force to maintain relationships between predetermined embedding vectors and a plurality of perturbation vectors generated based thereon, thereby preventing overfitting of a model and improving generalization performance.

In addition, a loss calculation module LCM according to an embodiment of the present disclosure may be a module that calculates various loss functions based on various vectors obtained through the multi-tasking learning model MtLM.

In an embodiment, the loss calculation module LCM may calculate a regression loss, an autoencoder loss, a consistency loss, a mapping loss, a distance loss, and/or an integrated loss according to an embodiment of the present disclosure. This will be described later in detail in the following multi-tasking model training method.

Accordingly, the loss calculation module LCM may support normalization and training for different parts of a model, and may provide feedback for model training to implement model optimization.

In one example, in an embodiment of the present disclosure, the multi-tasking learning model MtLM may execute model optimization and update through various data processing processes in association with the above-described modules.

As an example, the multi-tasking learning model MtLM may execute model optimization and parameter update by interoperating with the above-described modules based on an AdamW optimization algorithm or the like.

As described above, in an embodiment of the present disclosure, the multi-tasking learning model MtLM may not only learn knowledge data corresponding to various domains simultaneously but also efficiently learn relationships among those domains. Accordingly, the MtLM may perform effective multi-tasking learning that expands a learning area while simultaneously enabling collective learning of a local patterns associated with each domain and a common principle shared across the plurality of domains.

Accordingly, the multi-tasking learning model MtLM may directly enhance the processing performance and accuracy of various multi-tasking tasks based on the models trained as described above.

In one example, the multi-tasking learning model MtLM according to an embodiment of the present disclosure may include a sampler module SPM.

In this regard, although an embodiment of the present disclosure describes the multi-tasking learning model MtLM as including the sampler module SPM, the sampler module SPM may alternatively be implemented as a device and/or a server that is separate from the multi-tasking learning model MtLM according to some embodiments.

In detail, the sampler module SPM according to an embodiment of the present disclosure may be a module that executes sampling in a low-dimensional Gaussian space based on predetermined input data, and restores the sampled data to a high-dimensional space again to provide the restored data.

More specifically, the sampler module SPM according to an embodiment may be a module that converts predetermined input data into latent variables (hereinafter, low-dimensional latent variables) represented in a low-dimensional Gaussian space (i.e., a low-dimensional latent space), executes data sampling based on the converted low-dimensional latent variables, and provides data (hereinafter, high-dimensional latent variables) obtained by restoring the sampled low-dimensional latent variables (hereinafter, sampled latent variables) back to a high-dimensional space (i.e., a high-dimensional latent space).

Through such a sampler module SPM, in an embodiment, the multi-tasking learning model MtLM may provide output data (e.g., molecular structural formula data, etc.) in a valid form while processing given input data at low cost and with high efficiency. Further details regarding a method for sampling data for a general-purpose multi-tasking model and a method for providing a general-purpose multi-tasking model including the same will be described below.

In one example, the multi-tasking learning model MtLM according to an embodiment of the present disclosure may include a generation difficulty evaluation module GDM.

In this case, although an embodiment of the present disclosure describes the multi-tasking learning model MtLM as including the generation-difficulty evaluation module GDM, the generation difficulty evaluation module GDM may alternatively be implemented as a device and/or a server separate from the multi-tasking learning model MtLM according to some embodiments.

In detail, the generation difficulty evaluation module GDM according to an embodiment of the present disclosure may be a module that estimates and provides a density value for predetermined input data.

To this end, in an embodiment, the generation difficulty evaluation module GDM may be pre-trained based on a predetermined training data set.

The training data set (hereinafter, an evaluation module training set) for training the generation difficulty evaluation module GDM according to an embodiment may include a plurality of physical property input information (hereinafter, first training data), low-dimensional latent variables (hereinafter, second training data) according to each physical property input information, and/or high-dimensional latent variables (hereinafter, third training data) according to each physical property input information.

Accordingly, in an embodiment, the generation difficulty evaluation module GDM may learn overall data distribution (hereinafter, overall distribution of the first training data) based on the plurality of first training data included in the evaluation module training set, overall data distribution (hereinafter, overall distribution of the second training data) based on the plurality of second training data, and/or overall data distribution (hereinafter, overall distribution of the third training data) based on the plurality of third training data.

In this regard, in an embodiment, a specific method for the generation difficulty evaluation module GDM to learn overall data distribution according to the predetermined training data may be implemented based on various disclosed algorithms (e.g., a kernel density estimation (KDE) algorithm, a Gaussian mixture model (GMM), a radial basis function network (RBFN), a variational autoencoder (VAE), a generative adversarial network (GAN), a normalization flow, a k-nearest neighbor density estimation, a Dirichlet process mixture model (DPMM), a pixel CNN, and/or an energy-based model (EBM), etc.) capable of executing the above-described functional operation. However, for the sake of effective description, in the following description, it is described that the generation difficulty evaluation module GDM executes the above-described functional operation through the kernel density estimation (KDE) algorithm.

In this regard, for reference, the kernel density estimation algorithm may be an algorithm for estimating the density of overall data through a nonparametric method of estimating a continuous probability density function of data by applying a kernel function (generally, a Gaussian function) around a given data point.

In an embodiment, the generation difficulty evaluation module GDM may learn overall data distribution based on the evaluation module training set based on the kernel density estimation algorithm.

Accordingly, the generation difficulty evaluation module GDM may learn a data density according to the plurality of first training data included in the evaluation module training set (hereinafter, a first training data overall density), a data density according to the plurality of second training data (hereinafter, a second training data overall density), and/or a data density according to the plurality of third training data (hereinafter, a third training data overall density).

In addition, the generation difficulty evaluation module GDM may execute the above-described functional operation by further utilizing a marginal probability distribution estimation algorithm according to embodiments.

In this regard, for reference, the marginal probability distribution estimation algorithm is an algorithm for calculating a probability distribution of specific variables or a set of variables in a multi-dimensional probability distribution, and may be an algorithm for calculating a marginal probability of specific variables and integrating and analyzing influences of the remaining variables.

In addition, in an embodiment, the generation difficulty evaluation module GDM trained as described above may obtain predetermined input data.

The input data according to an embodiment may include predetermined physical property input information, low-dimensional latent variables according to the physical property input information, and/or high-dimensional latent variables according to the physical property input information.

In addition, in an embodiment, the generation difficulty evaluation module GDM may estimate and provide a density value for the obtained input data.

In particular, the generation difficulty evaluation module GDM may estimate and provide the density value for the obtained input data based on information pre-learned through the evaluation module training set.

In an embodiment, the generation difficulty evaluation module GDM may estimate and provide a density value (hereinafter, a first density value) for physical property input information of input data, a density value (hereinafter, a second density value) for low-dimensional latent variables according to the physical property input information, and/or a density value (hereinafter, a third density value) for high-dimensional latent variables according to the physical property input information.

In an embodiment, through such a generation difficulty evaluation module GDM, the multi-tasking learning model MtLM may calculate a validity index for input data based on a density value estimated for the given input data. A detailed description thereof will be made later in a method for providing a guide supporting performance enhancement of a multi-tasking model.

### [Multi-Tasking Model Pre-Training Method]

Hereinafter, a method by which the computing system 1000 according to an embodiment of the present disclosure executes transfer learning through geometric alignment in an integrated latent space for multi-tasks corresponding to a plurality of domains will be described in detail.

In general, existing transfer learning techniques are mainly focused on classifying image and/or language data sets, and have limitations in addressing regression problems or problems in non-Euclidean spaces.

In particular, when the training data set is insufficient, the degradation of the prediction performance for the above-described problems becomes even more inevitable, and when multi-tasking considering various task types is also required, the degradation of the performance in training and prediction therefor is further exacerbated.

In addition, because most of the existing methods are optimized for handling data in Euclidean spaces, they do not operate effectively in complex curved spaces or non-linear spaces.

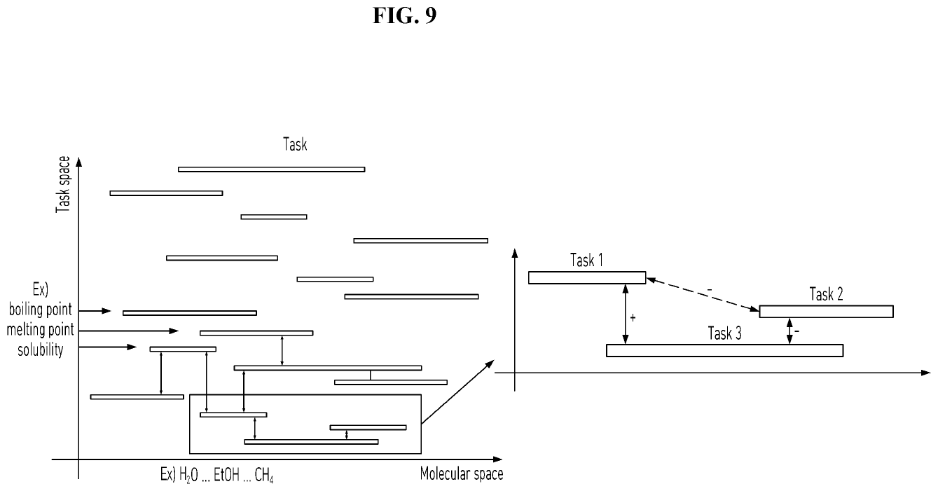

FIG. 9 illustrates an example of a conceptual diagram for describing a multi-tasking model pre-training method according to an embodiment of the present disclosure.

Accordingly, as shown in FIG. 9, the computing system 1000 according to an embodiment of the present disclosure is intended to provide a new multi-tasking model pre-training method capable of overcoming the regression problems of small-scale data sets and the limitations of the existing transfer learning techniques.

Hereinafter, in the description according to an embodiment of the present disclosure, for the sake of effective description, the above-described material will be limited to ‘molecules’, and domains thereof will be described based on ‘physical properties’.

This takes into account that molecular data sets generally have limited data, include various task types, and mainly deal with regression problems.

In particular, in the case of a molecular data set, various task processing associated with numerous physical properties is required, but data available for such processing are very limited, and the physical properties are closely related to or influenced by one another.

In consideration of these, a molecular data set may be data advantageous to be applied to multi-task processing according to a plurality of domains, and may be an example for the description of the multi-tasking model pre-training method according to an embodiment of the present disclosure.

However, the preset disclosure is not limited thereto, and it is obvious that any embodiment in which multi-tasks across multiple domains are applicable may be included within the scope of the present disclosure.

Hereinafter, a method for pre-training a multi-tasking model according to an embodiment of the present disclosure will be described in more detail with reference to the accompanying drawings.

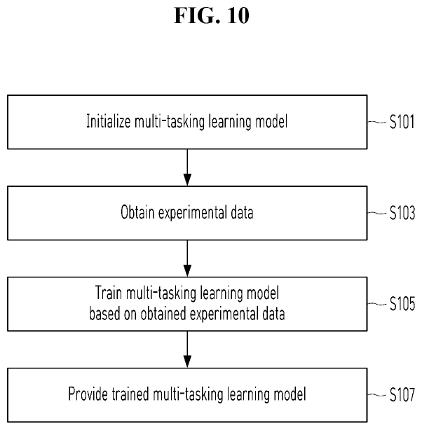

FIG. 10 is a block flowchart for illustrating a multi-tasking model pre-training method according to an embodiment of the present disclosure.

Referring to FIG. 10, a multi-tasking model pre-training method according to an embodiment of the present disclosure and a multi-tasking executing method using a machine learning model trained based thereon may include initializing the multi-tasking learning model MtLM (S101), obtaining experimental data (S103), training the multi-tasking learning model MtLM based on the obtained experimental data (S105), and providing the trained multi-tasking learning model MtLM (S107).

In detail, the computing system 1000 according to an embodiment of the present disclosure may initialize the multi-tasking learning model MtLM (S101).

Here, in other words, the multi-tasking learning model MtLM (geometrically aligned transfer encoder model) according to an embodiment of the present disclosure may be a machine learning model in which knowledge data (e.g., a latent vector, etc.) distributed across latent spaces for respective tasks are mutually aligned in a single integrated latent space M (manifold) through a geometric, thereby enabling processing of multi-task outputs across a plurality of domains.

In particular, the multi-tasking learning model MtLM according to an embodiment may not only learn knowledge data corresponding to various domains simultaneously but also efficiently learn relationships among those domains. Accordingly, the MtLM may perform effective multi-tasking learning that expands a learning area and simultaneously implements collective learning of a local pattern corresponding to each domain and a common principle shared across the plurality of domains.

In detail, in an embodiment, the computing system 1000 may execute initialization on each component included in the multi-tasking learning model MtLM as described above.

In an embodiment, the computing system 1000 may initialize an embedding network embedd(X), an encoder network fₑ, a regressor (head) network fₕ, a transfer network fₜ, and/or an inverse network fᵢ within the multi-tasking learning model MtLM with random parameters θ.

In addition, in an embodiment, the computing system 1000 may set a predetermined optimization algorithm to be applied to the multi-tasking learning model MtLM.

For example, the computing system 1000 may set a decoupled weight decay regularization (AdamW) algorithm as the optimization algorithm, and may enhance and use the optimization algorithm to independently process a weight decay according to embodiments.

In addition, the computing system 1000 according to an embodiment of the present disclosure may obtain the experimental data (S103).

Here, in other words, experimental data x according to an embodiment of the present disclosure may be training data used for training of the multi-tasking learning model MtLM, may include predetermined input data and output data (i.e., label) information corresponding thereto.

In an embodiment, such experimental data may include predetermined intrinsic material characteristic information and material physical property-specific information corresponding thereto. In particular, the experimental data may be configured such that predetermined intrinsic material characteristic information is used as input data, and material physical property-specific information is included as output data (i.e., label) mapped to the corresponding intrinsic material characteristic information.

The intrinsic material characteristic information according to an embodiment may refer to information specifying unique characteristics possessed by a predetermined material. In particular, in an embodiment, the intrinsic material characteristic information may specify unique characteristics possessed by predetermined molecules.

For example, the intrinsic material characteristic information may include at least one of a predetermined material name, a molecular structural formula, and/or chemical formula data. In the following description, the intrinsic material characteristic information is limited to molecular structural formula (e.g., n-dimensional (n≥2) molecular structural formula) data.

In addition, the material physical property-specific information according to an embodiment may be information specifying data values (i.e., characteristic values of physical properties, where the values include ranges) that a predetermined material has with respect to predetermined physical properties.

For example, the material physical property-specific information may include physical property (i.e., domain) values such as boiling point, melting point, refractive index, solubility, viscosity, surface tension, density, strength, and/or thermal conductivity of a predetermined material.

In other words, in the following embodiment, molecular structural formula data indicating the intrinsic material characteristic information will be used as input data, and physical property value data for each task will be used as output data.

In detail, in an embodiment, the computing system 1000 may obtain the experimental data as described above based on predetermined user input and/or association with an external server.

In more detail, in an embodiment, the computing system 1000 may obtain physical property relationship data indicating relationships among predetermined physical properties.

Specifically, in an embodiment, the computing system 1000 may obtain the physical property relationship data based on user input (e.g., an input of data manually collected by a user) and/or association with a predetermined artificial intelligence model.

In an embodiment, the computing system 1000 may obtain the physical property relationship data based on prompt engineering in association with a predetermined pre-trained large language model.

In detail, the computing system 1000 may execute data searches according to keywords for physical properties from a database of predetermined professional materials (e.g., papers, patents, and/or academic data) through a specific large language model.

In addition, the computing system 1000 may extract at least one piece of information indicating relationships among predetermined physical properties from the searched data.

In addition, the computing system 1000 may execute an editing process of classifying, characterizing, and organizing the extracted physical property relationship information according to criteria such as a physical property type and/or a relationship type.

Accordingly, the computing system 1000 may obtain physical property relationship data based on the edited information.

In addition, in an embodiment, the computing system 1000 may database the obtained physical property relationship data.

In detail, the computing system 1000 according to an embodiment may include a physical property relationship data database.

Here, the above-described physical property relationship data database may store data including information on relationships among predetermined physical properties.

Such a physical property relationship data database may store data manually collected by a person, or may store data automatically searched, extracted, and edited through a pre-trained artificial intelligence model.

In particular, in an embodiment, the computing system 1000 may store and manage the physical property relationship data obtained as described above in the physical property relationship data database.

Therefore, the computing system 1000 may construct the physical property relationship data database including the information on the relationships among various physical properties.

Thereafter, in an embodiment, the computing system 1000 may obtain physical property relationship information specifying relationships among different physical properties, based on the physical property relationship data included in the physical property relationship data database.

In an embodiment, the computing system 1000 may obtain at least one piece of physical property relationship information according to predetermined physical property relationship data by interoperating with a predetermined pre-trained large language model.

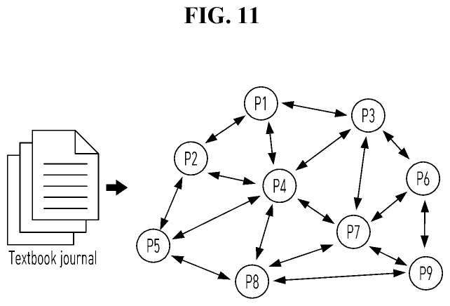

FIG. 11 illustrates an example of a knowledge graph showing relationships among physical properties according to an embodiment of the present disclosure.

For example, referring to FIG. 11, the computing system 1000 may obtain a predetermined knowledge graph as the physical property relationship information. In this case, the knowledge graph may include relationship information among first through n-th physical properties, including, for instance, relationship information between a first physical property P1 and a second physical property P2, relationship information between the first physical property P1 and a third physical property P3, and the like.

As a specific example, the physical property relationship information may include information regarding physical properties associated with a particular physical property, information on attributes of relationships (e.g., conflicting, similar, correlated, cause-and-effect, independent, proportional, and/or inversely related relationships), and/or information on a degree of association (association strength) corresponding to the determined relationship attributes.

Here, the attributes of relationships (hereinafter, physical property relationship attributes) and the degree of association information according to an embodiment may be represented in the knowledge graph by distinguishing numerical values indicating a positive correlation and numerical values indicating a negative correlation.

In addition, in an embodiment, the computing system 1000 may obtain the above-described experimental data based on the obtained physical property relationship information.

In particular, in an embodiment, the computing system 1000 may obtain the experimental data including predetermined intrinsic material characteristic information and material physical property-specific information corresponding thereto based on the various physical property relationship information obtained as described above.

In an embodiment, the computing system 1000 may obtain at least one piece of experimental data according to predetermined physical property relationship information by interoperating with a predetermined pre-trained large language model.

It has been described that the computing system 1000 obtains the experimental data through predetermined data pre-processing based on the given physical property relationship information, but this is only an example, and various embodiments are available, such as using the given physical property relationship information itself as the experimental data.

As a specific example, the computing system 1000 may obtain first experimental data including information on a first physical property with respect to molecular structural formulas of a plurality of materials, second experimental data including information on a second physical property with respect to molecular structural formulas of a plurality of materials, and the like.

Here, a plurality of materials corresponding to the first experimental data and a plurality of materials corresponding to the second experimental data may be different from each other or may be at least partially the same as each other.

As such, in an embodiment, the computing system 1000 may obtain the above-described experimental data based on physical property relationship information collected from various data sources, thereby implementing model training using more abundant training data and minimizing errors due to data bias and data shortage.

Accordingly, the computing system 1000 may maximize the utility of the various data sources to enable the trained model to execute more accurate and reliable prediction, and may directly enhance multi-task processing and prediction performance for a plurality of physical properties.

In this regard, in an embodiment of the present disclosure, the computing system 1000 may reflect the physical property relationship information (e.g., the degree of association information, etc.) obtained as described above in weights for at least one of various losses used during training of the multi-tasking learning model MtLM in step S205 to be described below.

In particular, in an embodiment, the computing system 1000 may execute effective multi-tasking learning that implements collective learning of a local pattern for each domain (i.e., physical property) and a common principle shared across a plurality of domains by reflecting physical property relationship information specifying relationships among various physical properties.

In one example, according to embodiments, when the relationship between the predetermined first physical property and the predetermined second physical property is represented as a mathematical formula in the physical property relationship data database, the computing system 1000 may detect the same.

In addition, the computing system 1000 may augment an experimental data set for training the multi-tasking learning model MtLM using the detected mathematical formula.

As an example, the computing system 1000 may expand a data set in which only the first physical property exists for a predetermined material into a data set that further considers a second physical property by applying data augmentation based on the detected mathematical formula.

Alternatively, the computing system 1000 may optimize and update the multi-tasking learning model MtLM using the detected mathematical formula as described above according to embodiments.

For example, when the multi-tasking learning model MtLM has learned only the first physical property between the first physical property and the second physical property, the computing system 1000 may update the multi-tasking learning model MtLM to predict the second physical property by applying the detected mathematical formula to the first physical property.

Alternatively, the computing system 1000 may enhance integrated latent space M mapping accuracy during transfer learning of the multi-tasking learning model MtLM using the detected mathematical formula according to embodiments.

For example, when the multi-tasking learning model MtLM has learned both of two tasks for predicting the first physical property and the second physical property, the computing system 1000 may re-execute transfer learning between a model for the task of predicting the first physical property and a model for the task of predicting the second physical property based on the detected mathematical formula. Accordingly, the computing system 1000 may enhance optimization of the mapping to the integrated latent space M in the multi-tasking learning model MtLM, thereby enhancing prediction accuracy.

In addition, the computing system 1000 according to an embodiment of the present disclosure may train the multi-tasking learning model MtLM based on the obtained experimental data (S105).

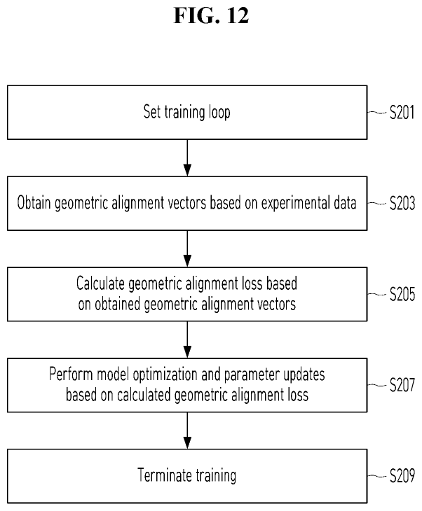

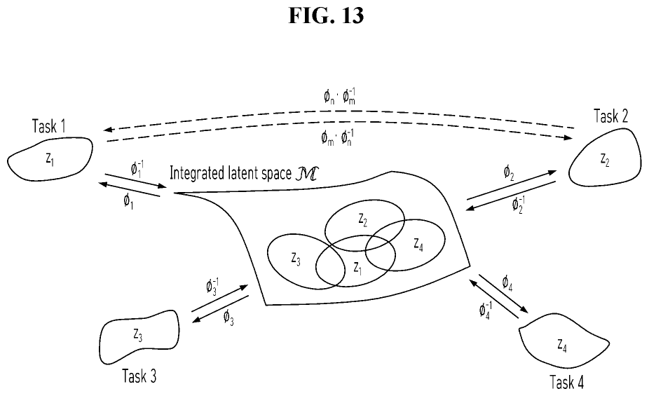

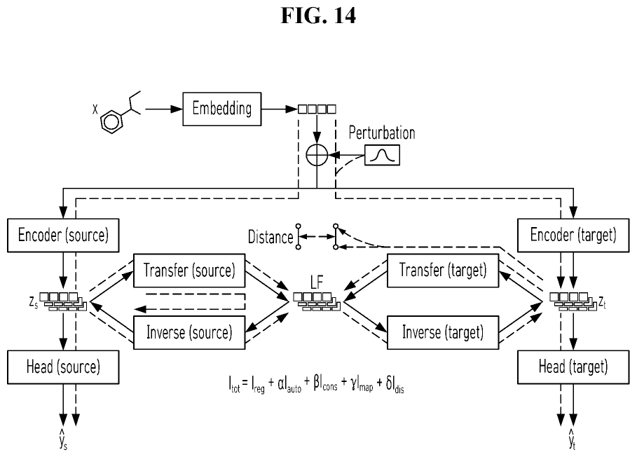

FIG. 12 is a block flowchart for describing a multi-tasking learning model MtLM training method according to an embodiment of the present disclosure, FIG. 13 illustrates an example of a first conceptual diagram for describing a multi-tasking learning model MtLM training method according to an embodiment of the present disclosure, and FIG. 14 illustrates an example of a second conceptual diagram for describing a multi-tasking learning model MtLM training method according to an embodiment of the present disclosure.

In particular, referring to FIGS. 12, 13, and 14, in an embodiment, the computing system 1000 may execute pre-training of the multi-tasking learning model MtLM based on the experimental data obtained as described above.

In this regard, in an embodiment, the computing system 1000 may allow the multi-tasking learning model MtLM to simultaneously learn multi-tasks for predicting at least two domains (i.e., physical properties).

In detail, in an embodiment, when the first task for executing prediction on the first physical property is referred to as a source task and the n-th task for executing prediction on the n-th (n>=2) physical property is referred to as a target task, the computing system 1000 may collectively execute transfer learning-based pre-training on the source task and the target task, and the reverse may also be executed in the same manner.

In other words, in an embodiment, when considering n physical properties, the computing system 1000 may simultaneously execute multiple transfer learning processes corresponding to n*n combinations among the first to n-th physical properties based on the multi-tasking learning model MtLM.

Accordingly, the computing system 1000 according to an embodiment of the present disclosure may allow the multi-tasking learning model MtLM to naturally learn correlations among physical properties learned together while learning the plurality of physical properties through transfer learning, thereby enabling more accurate prediction for each physical property.

As a specific example, the computing system 1000 may pre-train the multi-tasking learning model MtLM to learn relationships among the first to third physical properties based on experimental data including relationship information among the predetermined first to third physical properties. Thereafter, the computing system 1000 may more accurately predict relationship information among the first through third physical properties for a first material that has relationship information only for the first and second physical properties, using the multi-tasking learning model MtLM pre-trained as described above.

In addition, accordingly, when experimental data for each physical property includes data of different molecular structure types, the computing system 1000 may train the multi-tasking learning model MtLM for various molecular structure types through transfer learning, thereby implementing task processing that operates robustly against variations in molecular structures and provides accurate prediction values for physical properties.

In particular, the computing system 1000 according to an embodiment may execute pre-training that collectively trains the multi-tasking learning model MtLM on not only various physical property data but also relationships among various physical properties, thereby implementing effective multi-tasking learning that expands a model learning area and implements simultaneous/collective learning of a local pattern according to each physical property and a common principle shared across a plurality of physical properties.

Accordingly, the computing system 1000 may significantly enhance processing performance and quality of various multi-tasking tasks based on the multi-tasking learning model MtLM pre-trained as described above, thereby expanding a range in which accurate prediction is available.

Returning to the description, in an embodiment, the computing system 1000 may execute the transfer learning-based pre-training as described above according to the following processes.

In detail, in an embodiment, the computing system 1000 may set a training loop for the multi-tasking learning model MtLM (S201).

In more detail, in an embodiment, the computing system 1000 may set the number of repetitions of epochs, tasks, and/or batches during training.

In an embodiment, the computing system 1000 may set a training loop that repeats training for epochs ‘i’ from 1 to n (where n≥1), repeat training over each task ‘t’, and repeat training over each preset batch ‘b’.

In addition, in an embodiment, the computing system 1000 may obtain geometric alignment vectors based on the experimental data obtained as described above (S203).

Here, the geometric alignment vectors according to an embodiment of the present disclosure may refer to various vectors obtained through the multi-tasking learning model MtLM.

In an embodiment, the geometric alignment vectors may include embedding vectors a, perturbation vectors {ā}, encoding vectors, transfer vectors, inverse vectors, and the like.

In detail, in an embodiment, the computing system 1000 may input the obtained experimental data to the multi-tasking learning model MtLM.

In addition, in an embodiment, the computing system 1000 may obtain 1) embedding vectors based on the multi-tasking learning model MtLM to which the experimental data is input.

In more detail, the computing system 1000 may convert the input experimental data into the embedding vectors through an embedding network by interoperating with the embedding module EBM of the multi-tasking learning model MtLM.

Accordingly, the computing system 1000 may obtain the embedding vectors converted into a vector format by projecting the experimental data into a predetermined embedding space.

In addition, in an embodiment, the computing system 1000 may generate 2) perturbation vectors based on the obtained embedding vectors.

In detail, in an embodiment, the computing system 1000 may generate a plurality of perturbation vectors (i.e., perturbation points) on a predetermined periphery of the obtained embedding vectors by interoperating with the perturbation module PBM of the multi-tasking learning model MtLM.

In this regard, in an embodiment, the computing system 1000 may repeatedly execute the above-described functional operation for each task to obtain perturbation vectors corresponding to each task.

In an embodiment, the computing system 1000 may obtain a plurality of task-specific perturbation vectors including a perturbation vector corresponding to a task ‘t’ and a perturbation vector corresponding to a task ‘s’.

In addition, in an embodiment, the computing system 1000 may obtain encoding vectors, transfer vectors, and inverse vectors based on the generated perturbation vectors and embedding vectors.

In detail, referring to FIGS. 8, 13, and 14, in an embodiment, the computing system 1000 may obtain encoding vectors, transfer vectors, and inverse vectors for each task processing unit TPU by collectively interoperating with a plurality of task processing units TPU included in the multi-tasking learning model MtLM.

In particular, the computing system 1000 may obtain encoding vectors, transfer vectors, and inverse vectors for each of a plurality of tasks processed by each task processing unit TPU by collectively interoperating with a plurality of task processing units TPU respectively corresponding to a plurality of physical properties (i.e., domains) to be predicted.

Hereinafter, for the sake of effective description, a method of obtaining the vectors based on the task ‘t’ processed by the first task processing unit TPU and the task ‘s’ processed by the second task processing unit TPU will be described. However, all of the plurality of task processing units TPU described above may obtain the vectors for each of the plurality of tasks corresponding to each unit in the same manner as described below.

In detail, in an embodiment, the computing system 1000 may obtain 3) encoding vectors based on the generated perturbation vectors and embedding vectors.

Here, the encoding vectors according to an embodiment may include perturbation latent vectors that are latent vectors generated based on predetermined perturbation vectors, and original latent vectors generated based on embedding vectors that are original vectors of the perturbation vectors.

In more detail, in an embodiment, the computing system 1000 may project the generated perturbation vectors into a latent space corresponding to a respective task through the encoder network, and may convert the resulting perturbation vectors into latent vectors by interoperating with the encoder modules ECM included in the first and second task processing units TPU (hereinafter, training task processing units) of the multi-tasking learning model MtLM.

In addition, in an embodiment, the computing system 1000 may project the obtained embedding vectors to a latent space corresponding to the task through the encoder network and convert the resultant embedding vectors into latent vectors by interoperating with the encoder module ECM of the multi-tasking learning model MtLM.

Accordingly, in an embodiment, the computing system 1000 may obtain the perturbation latent vectors and the original latent vectors.

In this regard, in an embodiment, the computing system 1000 may repeatedly execute the above-described functional operation for each task to obtain the original latent vectors and the perturbation latent vectors corresponding to each task.

In an embodiment, the computing system 1000 may obtain an original latent vector zₜ (hereinafter, a t-th original latent vector) corresponding to the task ‘t’ and a perturbation latent vector {z̅ₜ̅} (hereinafter, a t-th perturbation latent vector) corresponding to the task ‘t’.

In addition, the computing system 1000 may obtain an original latent vector zₛ (hereinafter, an s-th original latent vector) corresponding to the task ‘s’ and a perturbation latent vector {z̅ₛ̅} (hereinafter, an s-th perturbation latent vector) corresponding to the task ‘s’.

In addition, in an embodiment, the computing system 1000 may obtain 4) transfer vectors based on the obtained encoding vectors.

Here, the transfer vector according to an embodiment may include perturbation transfer vectors that are transfer vectors generated based on predetermined perturbation latent vectors, and original transfer vectors that are transfer vectors generated based on original latent vectors corresponding to the perturbation latent vectors.

In detail, in an embodiment, the computing system 1000 may map the obtained perturbation latent vectors and original latent vectors to a latent space of another task (e.g., the task ‘s’ or the task ‘t’) through a transfer network and convert the mapped result into transfer vectors by interworking with the transfer module TFM included in the training task processing unit of the multi-tasking learning model MtLM.

Accordingly, the computing system 1000 may obtain the perturbation transfer vectors and the original transfer vectors.

In this regard, in an embodiment, the computing system 1000 may repeatedly execute the above-described functional operation for each task to obtain the original transfer vectors and the perturbation transfer vectors corresponding to each task.

In an embodiment, the computing system 1000 may obtain an original transfer vector mₜ (hereinafter, a t-th original transfer vector) corresponding to the task “t” and a perturbation transfer vector {m̅ₜ̅} (hereinafter, a t-th perturbation transfer vector) corresponding to the task “t”.

Further, the computing system 1000 may obtain an original transfer vector mₛ (hereinafter, an s-th original transfer vector) corresponding to the task “s” and a perturbation transfer vector {m̅ₛ̅} (hereinafter, an s-th perturbation transfer vector) corresponding to the task “s”.

Therefore, in an embodiment, the computing system 1000 may obtain experimental data-based geometric alignment vectors (i.e., embedding vectors, perturbation vectors, encoding vectors (including original latent vectors and perturbation latent vectors), and transfer vectors (including original transfer vectors and perturbation transfer vectors).

In addition, in an embodiment, the computing system 1000 may obtain 5) inverse vectors based on the obtained transfer vectors.

Here, the inverse vectors according to an embodiment may include perturbation inverse vectors that are inverse vectors generated based on predetermined perturbation transfer vectors, and original inverse vectors that are inverse vectors generated based on original transfer vectors corresponding to the perturbation transfer vectors.

In detail, in an embodiment, the computing system 1000 may reconstruct the obtained perturbation transfer vectors and original transfer vectors through an inverse network so as to be mapped back to an original latent space and convert the reconstructed vectors into inverse vectors by interoperating with the inverse module ITM included in the training task processing unit of the multi-tasking learning model MtLM.

Therefore, the computing system 1000 may obtain the perturbation inverse vectors and the original inverse vectors.

In this regard, in an embodiment, the computing system 1000 may repeatedly execute the above-described functional operation for each task to obtain original inverse vectors and perturbation inverse vectors corresponding to each task.

In an embodiment, the computing system 1000 may obtain an original inverse vector { [symbol] } (hereinafter, a t-th original inverse vector) corresponding to the task “t” and a perturbation inverse vector z′ₜ (hereinafter, a t-th perturbation inverse vector) corresponding to the task “t”.

In addition, the computing system 1000 may obtain an original inverse vector { [symbol] } (hereinafter, an s-th original inverse vector) corresponding to the task ‘s’ and a perturbation inverse vector z′ₛ (hereinafter, an s-th perturbation inverse vector) corresponding to the task ‘s’.

Therefore, in an embodiment, the computing system 1000 may obtain experimental data-based geometric alignment vectors (i.e., embedding vectors, perturbation vectors, encoding vectors (including original latent vectors and perturbation latent vectors), transfer vectors (including original transfer vectors and perturbation transfer vectors), and inverse vectors (including original inverse vectors and perturbation inverse vectors).

Further, in an embodiment, the computing system 1000 may calculate a geometric alignment loss based on the obtained geometric alignment vectors (S205).

Here, the geometric alignment loss according to an embodiment of the present disclosure may refer to various loss functions (Loss) calculated based on various vectors (i.e., geometric alignment vectors) obtained through the multi-tasking learning model MtLM.

In an embodiment, the geometric alignment loss may include a regression loss L_{reg}, an autoencoder loss Lₐᵤₜₒ, a consistency loss L_{cons}, a mapping loss Lₘₐₚ, a distance loss L_{dis}, and/or an integrated loss Lₜₒₜ.

In detail, in an embodiment, the computing system 1000 may calculate the geometric alignment loss based on the geometric alignment vectors obtained as described above (i.e., the geometric alignment vectors corresponding to a plurality of task processing units TPU and to a plurality of tasks processed by the task processing units TPU).

As described above, for the sake of effective description, a method of calculating the geometric alignment loss will be described below based on the task ‘t’ processed by the first task processing unit TPU and the task ‘s’ processed by the second task processing unit TPU (with particular emphasis on the task ‘t’).

In this regard, in an embodiment, the computing system 1000 may train the multi-tasking learning model MtLM by reflecting the physical property relationship information described in operation S103 described above in weights for at least one loss among various losses included in the geometric alignment loss.

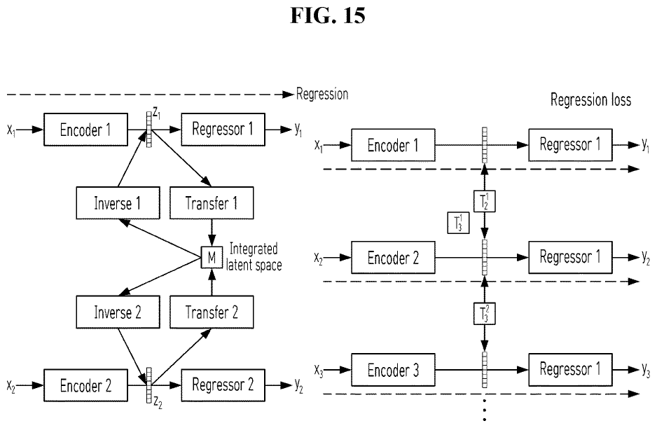

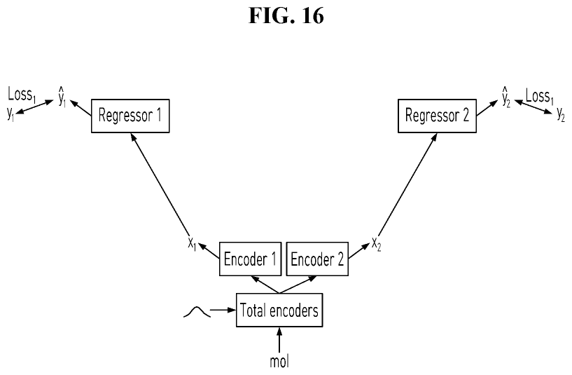

FIGS. 15 and 16 illustrate examples of diagrams for describing a regression loss calculation method according to an embodiment of the present disclosure.

In more detail, referring to FIGS. 14, 15, and 16, in an embodiment, the computing system 1000 may calculate 1) a regression loss based on the multi-tasking learning model MtLM that has obtained the geometric alignment vectors.

In more detail, in an embodiment, the computing system 1000 may calculate a regression loss based on a predicted value [symbol] predicted through the regressor module RGM and an actual value yₜ (i.e., a label value) according to the following [Mathematical formula 1]. Here, the predicted value in [Mathematical formula 1] may be represented as ‘fₕ(zₜ)’.

$$[ Mathematical formula 1 ]$$

$${L}_{reg} = MSE ( \hat{{y}_{r}} , {y}_{t} )$$

In particular, the computing system 1000 may calculate the regression loss by calculating a mean squared error (MSE) between the predicted value and the actual value.

In this regard, in an embodiment, each task may calculate an independent regression loss based on an encoder module ECM and a regressor module RGM matching each task and execute learning based thereon, thereby preventing mutual interference.

The computing system 1000 may calculate the regression loss as such, thereby easily evaluating regression performance of a model.

Further referring to FIG. 14, in an embodiment, the computing system 1000 may calculate 2) an autoencoder loss based on the multi-tasking learning model MtLM that has obtained the geometric alignment vectors.

In detail, in an embodiment, the computing system 1000 may calculate an autoencoder loss based on an original latent vector and an original inverse vector according to the following [Mathematical Formula 2].

$$[ Mathematical formula 2 ]$$

$${L}_{auto} = MSE ( \hat{{z}_{r}} , {z}_{t} )$$

In particular, the computing system 1000 may calculate the autoencoder loss by calculating a mean squared error (MSE) between the latent vector and the inverse vector.

In an embodiment, the computing system 1000 may enhance accuracy in a data transfer process through the autoencoder loss calculated as described above.

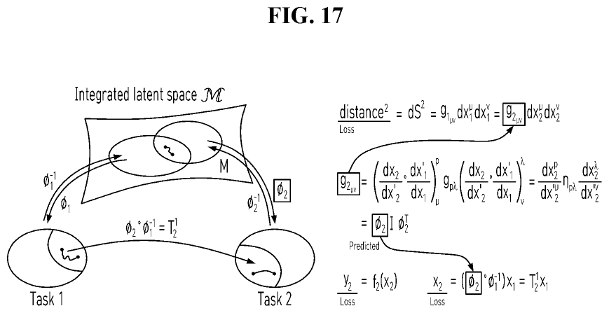

FIG. 17 illustrates an example of a diagram for describing an integrated latent space M mapping method according to an embodiment of the present disclosure.

Referring to FIG. 17, in an embodiment, the computing system 1000 may learn a bidirectional transformation matrix (TM) that enables mapping to a common integrated latent space M for each task.

In detail, in an embodiment, the computing system 1000 may connect latent spaces of tasks using knowledge data that hold all labels for both tasks.

In this process, the computing system 1000 may calculate a consistency loss and a mapping loss according to an embodiment.

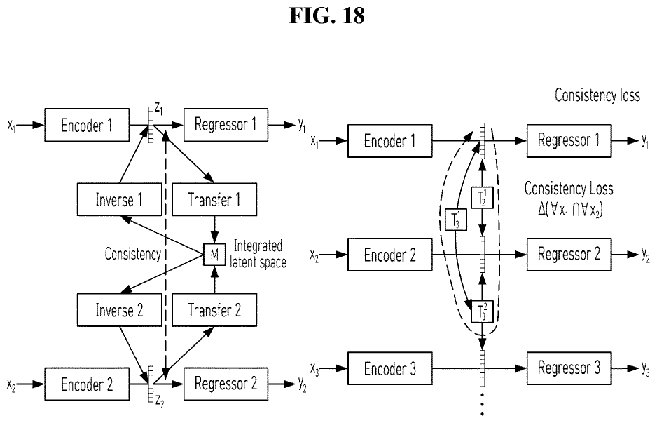

FIGS. 18 and 19 illustrate examples of diagrams for describing a consistency loss calculation method according to an embodiment of the present disclosure.

In more detail, referring to FIGS. 14, 18, and 19, in an embodiment, the computing system 1000 may calculate 3) a consistency loss based on the multi-tasking learning model MtLM that has obtained the geometric alignment vectors.

In detail, in an embodiment, the computing system 1000 may calculate a consistency loss based on the perturbation transfer vector of the task ‘t’ and the perturbation transfer vector of the task ‘s’ according to the following [Mathematical Formula 3].

$$[ Mathematical formula 3 ]$$

$${L}_{cons} = MSE ( \{ \overline{{m}_{s}} \} , \{ \overline{{m}_{t}} \} )$$

In particular, the computing system 1000 may calculate the consistency loss by calculating the mean squared error (MSE) between the t-th perturbation transfer vector and the s-th perturbation transfer vector.

In this regard, in an embodiment, the computing system 1000 may derive a metric for calculating a distance in space from the transformation matrix (TM), and may execute learning such that distances in the latent spaces of respective tasks become identical based on the derived metric.

Accordingly, the computing system 1000 may more effectively implement geometric alignment among tasks.

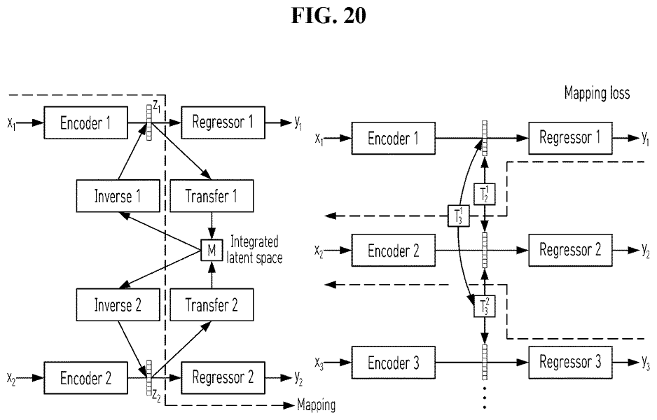

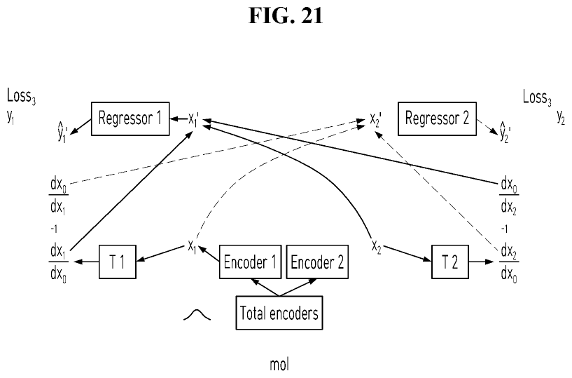

FIGS. 20 and 21 illustrate examples of diagrams for describing a mapping loss calculation method according to an embodiment of the present disclosure.

In addition, referring to FIGS. 14, 20, and 21, in an embodiment, the computing system 1000 may calculate 4) a mapping loss based on the multi-tasking learning model MtLM that has obtained the geometric alignment vectors.

In detail, in an embodiment, the computing system 1000 may calculate a mapping loss based on an actual value corresponding to the task ‘t’ and a predicted value based on an original inverse vector corresponding to the task ‘s’ according to the following [Mathematical formula 4].

$$[ Mathematical formula 4 ]$$

$${L}_{map} = MSE ( {f}_{h} ( {f}_{i} ( {m}_{s} ) ) , {y}_{t} )$$

In particular, the computing system 1000 may calculate the mapping loss by calculating a mean squared error (MSE) between the actual value of the task ‘t’ and the predicted value based on the original inverse vector of the task ‘s’.

In an embodiment, the computing system 1000 may implement learning in which latent vectors are transferred from a latent space of one task to a latent space of another task, and the other task is executed based on the transferred vectors, by calculating the mapping loss as described above. Through this process, latent characteristics may be induced to become similar to one another.

Accordingly, the computing system 1000 may evaluate the prediction performance of the vectors that have been transferred to the latent space of another task and induce learning in a direction to enhance the prediction performance.

Referring to FIG. 14, in an embodiment, the computing system 1000 may calculate 5) a distance loss based on the multi-tasking learning model MtLM that has obtained the geometric alignment vectors.

In detail, in an embodiment, the computing system 1000 may calculate an inter-task distance loss based on distances Sₜ (hereinafter, transfer vector displacements) between original transfer vectors and perturbation transfer vectors of each task according to the following [Mathematical Formula 5] and [Mathematical Formula 6].

In more detail, in an embodiment, the computing system 1000 may calculate a distance

$${s}_{i}^{t}$$

hereinafter, a t-th transfer vector displacement) between a t-th original transfer vector and a t-th perturbation transfer vector corresponding to a task “t” according to the following [Mathematical Formula (a) in 5].

In addition, the computing system 1000 may calculate a distance

$${s}_{i}^{s}$$

(hereinafter, an s-th transfer vector displacement) between an s-th original transfer vector and an s-th perturbation transfer vector corresponding to the task ‘s’ according to the following [Mathematical Formula (b) in 5].

$$\begin{matrix}[ Mathematical Formula 5 ] & \\ {s}_{i}^{s} = {m}_{t} - \{ \overline{{m}_{t}} \} & ( a )\end{matrix}$$

$${s}_{i}^{t} = {m}_{s} - \{ \overline{{m}_{s}} \} \qquad ( b )$$

In addition, in an embodiment, the computing system 1000 may calculate the distance loss by calculating a mean squared error (MSE) between the t-th transfer vector displacement and the s-th transfer vector displacement according to the following [Mathematical Formula 6].

$$[ Mathematical Formula 6 ]$$

$${L}_{dis} = \frac{1}{M} {\sum }_{i} MSE ( {s}_{i}^{s} , {s}_{i}^{t} )$$

Here, ‘M’ in [Mathematical Formula 6] refers to the number of perturbation points.

In this regard, in an embodiment, the computing system 1000 may define the t-th transfer vector displacement and the s-th transfer vector displacement as displacements in a source task and a target task, respectively.

Accordingly, the computing system 1000 may interpret the t-th transfer vector displacement and the s-th transfer vector displacement as being located within a flat Euclidean space, thereby enabling easier calculation of the distance between the original transfer vector and the perturbation transfer vector.

Accordingly, the computing system 1000 may support maintaining consistency with respect to the latent space of the model in a more complete manner.

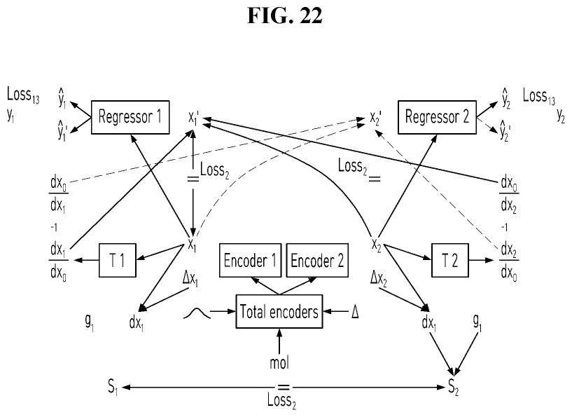

FIG. 22 illustrates an example of a diagram for describing an integrated loss calculation method according to an embodiment of the present disclosure.

In addition, referring to FIGS. 14 and 22, in an embodiment, the computing system 1000 may calculate 6) an integrated loss based on the multi-tasking learning model MtLM that has obtained the geometric alignment vectors.

In detail, in an embodiment, the computing system 1000 may calculate an integrated loss obtained by a weighted sum of the above-described regression loss, autoencoder loss, consistency loss, mapping loss, and distance loss, according to the following [Mathematical Formula 7].

$$[ Mathematical Formula 7 ]$$

$${L}_{tot} = {L}_{reg} + α {L}_{auto} + β {L}_{cons} + γ {L}_{map} + δ {L}_{dis}$$

In this regard, in an embodiment, the computing system 1000 may apply a weight for each loss function so that each loss function may be optimized for a specific aspect of the model.

Here, ‘α’ in [Mathematical Formula 7] denotes a weight of the autoencoder loss, ‘β’ denotes a weight of the consistency loss, ‘γ’ denotes a weight of the mapping loss, and ‘δ’ denotes a weight of the distance loss.

In an embodiment, the computing system 1000 may update parameters in a direction of minimizing the integrated loss by adjusting the importance of the loss function corresponding to each weight in the training process of the model by using the above-described weights.

In this regard, in an embodiment, the computing system 1000 may adjust a weight for at least one loss included in the integrated loss according to the physical property relationship information obtained in operation S103 described above.

In an embodiment, the computing system 1000 may determine or adjust a value of the weight of the mapping loss, which is a loss supporting learning of relationships among physical properties, by reflecting the above-described physical property relationship information.

As a specific example, when a predetermined first physical property and a predetermined second physical property exhibit a correlation and a high degree of association with each other, the computing system 1000 may reflect the physical property relationship information by increasing the weight of the mapping loss, thereby enhancing learning based on the mapping loss.

As another example, when the predetermined first physical property and the second physical property do not have a correlation or have a low degree of association with each other, the computing system 1000 may reflect the physical property relationship information in a manner of weakening learning based on the mapping loss by reducing the weight of the mapping loss.

Accordingly, the computing system 1000 may more accurately apply the physical property relationship information detected from the previously published professional materials from various sources to training of the multi-tasking learning model MtLM.

Returning back to FIG. 12, in an embodiment, the computing system 1000 may also execute model optimization and parameter updates based on the geometric alignment loss calculated as described above (S207).

In detail, in an embodiment, the computing system 1000 may execute optimization and parameter updates for the multi-tasking learning model MtLM based on the above-described integrated loss.

In an embodiment, the computing system 1000 may calculate a gradient based on the integrated loss for each parameter of the multi-tasking learning model MtLM through backpropagation.

Then, the computing system 1000 may execute parameter updates for the multi-tasking learning model MtLM using the calculated gradient and a preset optimization algorithm (e.g., the decoupled weight decay regularization (AdamW) algorithm, etc.).

Accordingly, the computing system 1000 may implement optimization of the multi-tasking learning model MtLM based on the geometric alignment loss (in particular, the integrated loss).

As described above, in an embodiment, the computing system 1000 may execute optimization and parameter update training of the multi-tasking learning model MtLM through a combination of various loss functions calculated in a multi-faceted manner.

In this regard, each loss function may easily assist in improving the performance of the model by adjusting the accuracy, consistency, and/or distance of knowledge data mapping.

Accordingly, the computing system 1000 may implement a multi-tasking model that provides enhanced performance by overcoming the regression problems of the small-scale data sets and the limitations of the existing transfer learning techniques, while operating more stably and providing enhanced generalization performance.

In addition, in an embodiment, the computing system 1000 may terminate training of the multi-tasking learning model MtLM (S209).

In detail, in an embodiment, the computing system 1000 may terminate the multi-tasking learning model MtLM training process described above when a preset training termination condition is satisfied.

In an embodiment, when the set training loop is completed, the computing system 1000 may terminate training of the multi-tasking learning model MtLM.

As described above, in an embodiment, the computing system 1000 may enable the multi-tasking learning model MtLM to simultaneously learn prediction tasks for at least two physical properties, thereby allowing the multi-tasking learning model MtLM to naturally learn, through transfer learning, correlations among the physical properties, which are learned together while learning the plurality of physical properties. As a result, the multi-tasking learning model MtLM may perform more accurate predictions for each physical property.

In addition, when experimental data for respective physical properties include data corresponding to different types of molecular structures, the computing system 1000 may train the multi-tasking learning model MtLM on various molecular structure types through transfer learning, Accordingly, the multi-tasking learning model MtLM may perform task processing that is robust to variations in molecular structures and may provide accurate predicted values for the physical properties.

In particular, the computing system 1000 according to an embodiment may execute pre-training that collectively trains the multi-tasking learning model MtLM on not only various physical property data but also relationships among multiple physical properties. Accordingly, the multi-tasking learning model MtLM may implement effective multi-tasking learning that expands a model learning area and simultaneously enables collective learning of a local pattern corresponding to each physical property and a common principle shared across a plurality of physical properties.

Accordingly, the computing system 1000 may significantly enhance processing performance and quality of various multi-tasking tasks based on the multi-tasking learning model MtLM pre-trained as described above, thereby expanding the range in which accurate prediction is available.

Returning to FIG. 10, the computing system 1000 according to an embodiment of the present disclosure may also provide the trained multi-tasking learning model MtLM (S107).

In particular, in an embodiment, the computing system 1000 may provide the multi-tasking learning model MtLM trained as described above in a predetermined method.

In an embodiment, the computing system 1000 may provide the multi-tasking learning model MtLM trained according to an embodiment of the present disclosure in association with a predetermined application service (e.g., a material synthesis/evaluation service, a material physical property prediction service, and/or an optimal material recommendation service).

Specifically, in an embodiment, the computing system 1000 may provide the multi-tasking learning model MtLM through a service that, upon receiving a predetermined molecular structural formula, inputs the corresponding molecular structural formula into the multi-tasking learning model MtLM and outputs physical property values for a plurality of domains (i.e., physical properties) pre-learned by the multi-tasking learning model MtLM. In this regard, each physical property value may be provided as a physical property value having the highest probability and/or a physical property value range corresponding to a specific probability.

On the contrary, according to embodiments, the computing system 1000 may provide the multi-tasking learning model MtLM through a service that inversely designs the pre-trained multi-tasking learning model MtLM, and when receiving a plurality of material physical property values, inputs the corresponding physical property values to the inversely designed multi-tasking learning model MtLM to output at least one molecular structural formula satisfying the physical property values.

As such, in an embodiment, the computing system 1000 may effectively support various multi-tasking task processing in various ways using the multi-tasking learning model MtLM having enhanced performance according to an embodiment of the present disclosure.

As described above, in an embodiment of the present disclosure, the computing system 1000 may enable mutual transfer and learning of knowledge data of a latent space for each task through geometric alignment in one integrated latent space in order to process multi-tasks for outputs corresponding to a plurality of domains, thereby providing the multi-tasking learning model MtLM that provides enhanced performance by overcoming the regression problems of the small-scale data sets and the limitations of the existing transfer learning techniques, while operating more stably.

Accordingly, the computing system 1000 may provide a transfer learning-based multi-tasking model that stably and robustly operates while exhibiting high generalization performance even in situations where an amount of given data is limited, where various task types are included, or where regression problems are mainly dealt.

In other words, even though a domain lacking experimental data (training data) exists among a plurality of domains (e.g., physical properties), the computing system 1000 may provide the multi-tasking learning model MtLM having enhanced prediction performance based on knowledge distilled through geometrical alignment-based transfer learning executed in association with other domains.

For example, when the multi-tasking learning model MtLM is pre-trained based on first through tenth physical properties for each of a plurality of molecular structural formulas and subsequently receives a first molecular structural formula that includes data only for the first through fifth physical properties, the computing system 1000 may more accurately predict respective values for the remaining sixth through tenth physical properties for the first molecular structural formula based on knowledge data transferred and distilled through the pre-learning, and may generate and provide output data based accordingly.

As described above, the computing system 1000 according to an embodiment of the present disclosure may provide a multi-tasking model that implements effective geometric alignment-based transfer learning, guarantees high generalization performance, enhances prediction accuracy for regression problems, supports normalization through combinations of various loss functions, and guarantees robust performance through a stable learning process.

As described above, a method and a system for training a multi-tasking model based on a data-centric technique according to an embodiment of the present disclosure may execute transfer learning through geometric alignment in an integrated latent space for multi-tasks corresponding to a plurality of domains, thereby accurately predicting an integrated output satisfying needs of the plurality of domains.

In addition, a method and a system for training a multi-tasking model based on a data-centric technique according to an embodiment of the present disclosure may simultaneously train various prediction tasks corresponding to a plurality of domains during a transfer learning process. Through this approach, the model may collectively learn not only individual principles of the respective domains, but also correlations among the domains and a common principle shared across all of the domains, thereby expanding a model-learning area and broadening a prediction-acceptance range for each domain.

Accordingly, a method and a system for training a multi-tasking model based on a data-centric technique according to an embodiment of the present disclosure may directly enhance performance and quality of processing various multi-tasking tasks using the trained model.

In addition, a method and a system for training a multi-tasking model based on a data-centric technique according to an embodiment of the present disclosure may implement mutual exchange of information by aligning geometric characteristics of the various prediction tasks, thereby easily supporting transfer of knowledge among mutually related data and enhancement of prediction performance resulted therefrom.

Furthermore, accordingly, a method and a system for training a multi-tasking model based on a data-centric technique according to an embodiment of the present disclosure may increase resistance to unnecessary interference information and increase stability of the model.

In addition, a method and a system for training a multi-tasking model based on a data-centric technique according to an embodiment of the present disclosure may secure data sets for training by utilizing source data from various sources, thereby increasing the diversity of data used for model training and allowing a model to learn more information to enhance learning performance.

In addition, a method and a system for training a multi-tasking model based on a data-centric technique according to an embodiment of the present disclosure may predict a plurality of physical properties for a specific material by applying the multi-tasking learning model MtLM trained as described above to relationship predictions between a plurality of physical properties and materials, and provide a multi-tasking model capable of predicting a specific material satisfying a plurality of physical properties, thereby improving overall quality across related industries by providing a multi-tasking model that may be universally utilized for various materials.

In addition, a method and a system for training a multi-tasking model based on a data-centric technique according to an embodiment of the present disclosure may transfer knowledge learned from a source task to a target task through transfer learning to solve data shortage problems, thereby providing a multi-tasking model that maintains high performance for multi-tasks even with small-scale data sets.

Therefore, a method and a system for training a multi-tasking model based on a data-centric technique according to an embodiment of the present disclosure may expand the range of applicability to fields in which application of machine learning models had previously been difficult due to insufficient data or domain knowledge.

In addition, a method and a system for training a multi-tasking model based on a data-centric technique according to an embodiment of the present disclosure may provide a specialized transfer learning technique that may be effectively applied to regression problems, thereby exhibiting high prediction performance even for complex regression problems such as molecular data sets.

In addition, a method and a system for training a multi-tasking model based on a data-centric technique according to an embodiment of the present disclosure may optimize knowledge transfer between a source task and a target task through a Riemann geometric approach, thereby maintaining geometric consistency between tasks and improving the efficiency of transfer learning.

In addition, a method and a system for training a multi-tasking model based on a data-centric technique according to an embodiment of the present disclosure may normalize various aspects of the model by combining multiple loss functions, thereby further improving generalization performance of the model.

[Method for sampling data for general-purpose multi-tasking model and method for providing general-purpose multi-tasking model including same]

Hereinafter, a method by which the computing system 1000 according to an embodiment of the present disclosure implements a method for sampling data for a general-purpose multi-tasking model and a method for providing a general-purpose multi-tasking model including the same (i.e., a general-purpose multi-tasking model provision service) will be described in detail with reference to the accompanying drawings.

As described above, hereinafter, the multi-tasking learning model MtLM will be described as operating by applying predetermined materials and physical properties as an example, but the present disclosure is not limited thereto.

The multi-tasking learning model MtLM according to the present embodiment may use characteristic values (i.e., physical property values) for respective N (1<=N<T) physical properties (i.e., N-dimensional physical properties) as input data, and may use predetermined molecular structural formula data satisfying the input N physical property values as output data.

Here, the above-described molecular structural formula data may include molecular structural formulas representing combinations of physical properties that satisfy the given N physical property values and are physically valid (e.g., having a physical feasibility equal to or greater than a predetermined criterion).

For reference, molecular structural formulas refer to data representing and storing structures such as compositions and binding methods of molecules, and may refer to data represented in various forms including chemical formulas, structural formulas, skeletal formulas, 3D structural formulas, and/or SMILES.

In the following embodiment, for the sake of effective description, it will be described that the multi-tasking learning model MtLM supports T (2<=T) physical properties (i.e., T-dimensional physical properties) in an embodiment, and generates and provides predetermined output data (e.g., molecular structural formula data, etc.) based on characteristic values (i.e., physical property values) for respective physical properties included in a set of T physical properties.

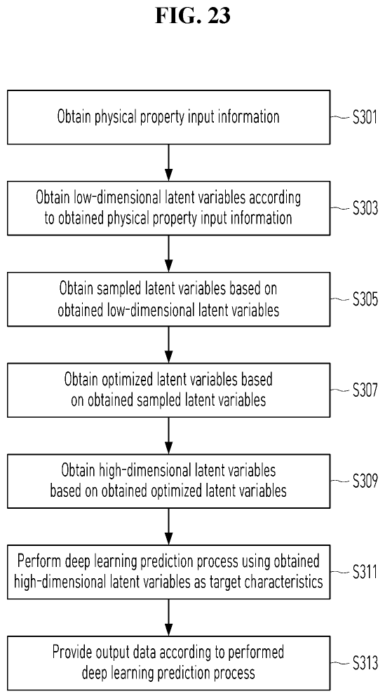

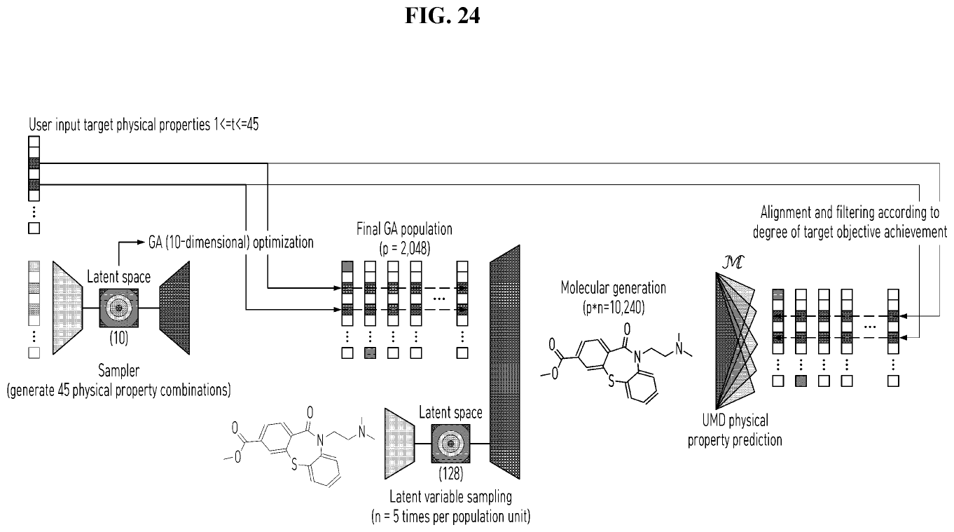

FIG. 23 illustrates a block diagram for describing a method for sampling data for a general-purpose multi-tasking model and a method for providing a general-purpose multi-tasking model including the same according to an embodiment of the present disclosure, and FIG. 24 illustrates an example of a conceptual diagram for describing a method for sampling data for a general-purpose multi-tasking model and a method for providing a general-purpose multi-tasking model including the same according to an embodiment of the present disclosure.

In detail, referring to FIGS. 23 and 24, a method for sampling data for a general-purpose multi-tasking model and a method for providing a general-purpose multi-tasking model including the same according to an embodiment of the present disclosure may include obtaining predetermined physical property input information (S301), obtaining low-dimensional latent variables according to the obtained physical property input information (S303), obtaining sampled latent variables based on the obtained low-dimensional latent variables (S305), obtaining optimized latent variables based on the obtained sampled latent variables (S307), obtaining high-dimensional latent variables based on the obtained optimized latent variables (S309), executing a deep learning prediction process using the obtained high-dimensional latent variables as target characteristics (S311), and providing output data according to the executed deep learning prediction process (S313).

In more detail, the computing system 1000 according to an embodiment of the present disclosure may obtain predetermined physical property input information (S301).

Here, the physical property input information according to an embodiment may be information obtained based on user input, including characteristic values (i.e., physical property values) for respective N physical properties (where 1≤N<T) (i.e., N-dimensional physical properties).

In more detail, in an embodiment, the computing system 1000 may obtain physical property input information including N physical property values (hereinafter, target physical property values) based on user input via a predetermined user interface.

In particular, the computing system 1000 may obtain physical property input information specifying physical property values that a user intends to satisfy with respect to output data (e.g., molecular structural formula data, etc.) of the multi-tasking learning model MtLM.

In addition, the computing system 1000 according to an embodiment of the present disclosure may obtain low-dimensional latent variables corresponding to the obtained physical property input information (S303).

Here, in other words, the low-dimensional latent variables according to an embodiment may refer to data obtained by projecting and transforming given input data into a low-dimensional Gaussian space.

In an embodiment, such low-dimensional latent variables may be represented as respective points existing in the low-dimensional Gaussian space.

In detail, in an embodiment, the computing system 1000 may project the physical property input information obtained as described above into the low-dimensional Gaussian space by interoperating with the above-described sampler module SPM.

Accordingly, the computing system 1000 may convert the physical property input information to be represented in an L-dimensional space (where 1≤L<N, T).

In particular, the computing system 1000 may execute a process of compressing a complex high-dimensional space into a low-dimensional space (e.g., the low-dimensional Gaussian space) that is simple and easy to sample, thereby executing conversion to represent given high-dimensional input data (i.e., physical property input information) in a low dimension.

In this regard, in an embodiment, the computing system 1000 may input the physical property input information to a predetermined encoding network.

Then, the encoding network may generate low-dimensional latent variables (e.g., means and/or variances) by approximating a distribution of the input physical property input information as a Gaussian distribution.

Specifically, the encoding network may learn a latent structure of the physical property input information through minimization of a reconstruction loss and a normalization loss. As a result, the encoding network may construct a latent space that conforms to a Gaussian distribution, thereby producing low-dimensional latent variables.

Then, the encoding network may provide the generated low-dimensional latent variables to the computing system 1000.

Accordingly, the computing system 1000 may obtain the low-dimensional latent variables, which are data in which high-dimensional physical property input information is represented in a low-dimensional Gaussian space.

In addition, the computing system 1000 according to an embodiment of the present disclosure may obtain sampled latent variables based on the obtained low-dimensional latent variables (S305).

Here, in other words, the sampled latent variables according to an embodiment may refer to data sampled based on the low-dimensional latent variables existing in the low-dimensional Gaussian space.

In an embodiment, such sampled latent variables may represent various combinations of physical properties based on respective points existing in the low-dimensional Gaussian space.

As an example, the computing system 1000 may execute data sampling in the low-dimensional Gaussian space using the low-dimensional latent variables such as means and/or variances.

Accordingly, the computing system 1000 may obtain the sampled latent variables based on the low-dimensional latent variables existing in the low-dimensional Gaussian space.

Accordingly, the computing system 1000 according to an embodiment may generate an initial population according to the sampled latent variables.

As described above, in an embodiment, the computing system 1000 may execute efficient latent variable sampling in a low-dimensional space in which data sampling is facilitated, thereby reducing analysis costs for various physical property combinations and executing faster data processing.

In addition, the computing system 1000 according to an embodiment of the present disclosure may obtain optimized latent variables based on the obtained sampled latent variables (S307).

Here, the optimized latent variables according to an embodiment may refer to sampled latent variables optimized through a genetic algorithm (GA).

In an embodiment, such optimized latent variables may include various combinations of physical properties and simultaneously satisfy target physical property values according to user needs.

Here, for reference, the genetic algorithm (GA) may refer to an optimization algorithm that finds an optimal solution by mimicking principles of natural selection and genetics. Such a genetic algorithm may be an algorithm that selects entities according to calculated fitness starting from an initial group, and progressively finds a better solution through crossover and mutation processes based on the selected entities.

In detail, in an embodiment, the computing system 1000 may execute GA-based optimization based on the obtained sampled latent variables.

In more detail, in an embodiment, the computing system 1000 may optimize the sampled latent variables in a direction that satisfies the above-described target physical property values using the genetic algorithm as described above.

In other words, the computing system 1000 may manipulate the sampled latent variables in the low-dimensional Gaussian space through the genetic algorithm such that, among T-dimensional physical property combinations included in the output data (e.g., molecular structural formula data, etc.) of the multi-tasking learning model MtLM, N physical properties according to user input are optimized to achieve target values.

Accordingly, in an embodiment, the computing system 1000 may obtain GA-optimized latent variables in the low-dimensional Gaussian space (i.e., the optimized latent variables).

In addition, accordingly, the computing system 1000 may obtain a population (hereinafter, optimal population) corresponding to the obtained optimized latent variables.

In more detail, in an embodiment, the computing system 1000 may execute fitness evaluation based on the initial population generated as the sampled latent variables are obtained.

In an embodiment, the computing system 1000 may input the sampled latent variables (the initial population) into a predetermined decoding network.

Then, the decoding network may restore the input sampled latent variables into a high-dimensional space.

Then, the decoding network may provide the sampled latent variables (i.e., a predetermined combination of physical properties) restored into the high-dimensional space to the computing system 1000.

Subsequently, the computing system 1000 that has obtained the sampled latent variables restored into the high-dimensional space through the decoding network may execute fitness evaluation for evaluating validity and degrees of satisfaction of target property values with respect to the obtained restored data.

In addition, in an embodiment, the computing system 1000 may select latent variables determined to have fitness (i.e., validity and degrees of satisfaction of target physical property values) equal to or higher than a predetermined reference as a result of the evaluation.

In addition, in an embodiment, the computing system 1000 may execute crossover on the selected latent variables and apply mutation to some of them to generate new latent variables.

This may be a process for detecting a more valid combination of physical properties by expanding a search in a low-dimensional Gaussian space.

In addition, in an embodiment, the computing system 1000 may derive optimal latent variables (i.e., optimized latent variables) by repeating the above-described processes (here, fitness evaluation, selection, and crossover and mutation processes) over several generations.

The computing system 1000 may gradually enhance fitness for each generation and finally derive optimized latent variables that satisfy target physical property values.

Accordingly, in an embodiment, the computing system 1000 may obtain optimized latent variables and a corresponding optimal population.

As such, through GA-based optimization, in an embodiment, the computing system 1000 may adjust output data (e.g., molecular structural formula data, etc.) of the multi-tasking learning model MtLM to satisfy N target physical property values according to user input.

In addition, the computing system 1000 according to an embodiment of the present disclosure may obtain high-dimensional latent variables based on the obtained optimized latent variables (S309).

Here, in other words, the high-dimensional latent variables according to an embodiment may refer to data obtained by restoring the optimized latent variables back into the high-dimensional space.

In an embodiment, such high-dimensional latent variables may form a combination of physical properties that is physically valid while satisfying N physical property values (i.e., target physical property values) desired by a user.

In detail, in an embodiment, the computing system 1000 may cooperate with the aforementioned sampler module SPM to project optimized latent variables, which reside in a low-dimensional (i.e., L-dimensional) Gaussian space into a high-dimensional (i.e., T-dimensional) space. Through this projection, the optimized latent variables may be expressed in the T-dimensional space.

In other words, the computing system 1000 may project L-dimensional Gaussian latent variables into a T-dimensional physical property set supported by the multi-tasking learning model MtLM.

Accordingly, the computing system 1000 may obtain high-dimensional latent variables, which are data obtained by projecting the optimized latent variables in the low-dimensional Gaussian space into the T-dimensional space supported by the multi-tasking learning model MtLM.

In more detail, in an embodiment, the computing system 1000 may input the optimized latent variables (optimal population) into a predetermined decoding network.

Then, the decoding network may restore the input optimized latent variables into a high-dimensional (here, T-dimensional) space.

The decoding network may restore the optimized latent variables into the T-dimensional space while maintaining correlations among respective physical properties.

Then, the decoding network may provide the optimized latent variables (i.e., the high-dimensional latent variables) restored into the high-dimensional (here, T-dimensional) space to the computing system 1000.

Accordingly, in an embodiment, the computing system 1000 may acquire data (e.g., high-dimensional latent variables) generated by projecting data from a T-dimensional space, which corresponds to T different types of physical properties, into an L-dimensional space (where 1≤L<N, T) for sampling and subsequently restoring the sampled data into the T-dimensional space.

In addition, in an embodiment, the computing system 1000 may obtain a population (hereinafter, a T-dimensional population) according to the obtained high-dimensional latent variables.

As such, in an embodiment, the computing system 1000 may restore the latent variables sampled and optimized in the low-dimensional Gaussian space into the high-dimensional physical property space while maintaining correlations among respective physical properties.

Accordingly, the computing system 1000 may support a combination of physical properties that takes into account complex interactions among sampled data by utilizing advantages of the high-dimensional space while executing low-cost/high-efficiency data (i.e., physical property value) sampling by utilizing advantages of the low-dimensional space.

Accordingly, the computing system 1000 may easily avoid unrealistic combinations of physical properties and effectively ensure implementation of physically valid combinations of physical properties.

In addition, the computing system 1000 may automatically generate natural combinations of physical properties including characteristic values of up to (T-N) physical properties not input by the user as the low-dimensional data is restored into a high-dimensional space as described above.

Accordingly, the computing system 1000 may support assigning characteristic values that are valid for generation of output data (e.g., molecular structural formula data, etc.) to physical properties that are not explicitly input by the user.

In other words, the computing system 1000 may automatically set characteristics of physical properties not input by the user to values valid for actual molecular structure formation and provide corresponding output data (e.g., molecular structural formula data, etc.), thereby ensuring physical validity of the provided combination of physical properties.

For example, when a user inputs N characteristic values (i.e., physical property values) for specific physical properties (e.g., thermal conductivity and/or strength) of first molecules, the computing system 1000 may execute sampling and GA-based optimization in a low-dimensional Gaussian space based thereon, and restore the same into high-dimensional data. As a result, among various physical properties required to generate the first molecules, the computing system 1000 may assign valid values to characteristic values of other physical properties (e.g., density and/or solubility) not input by the user, and may generate and provide a molecular structure in which all physical properties required to generate the first molecules are naturally combined by comprehensively reflecting the assigned characteristic values and the characteristic values of the physical properties input by the user.

In summary, in an embodiment, the computing system 1000 may sample latent variables within the low-dimensional Gaussian space, perform optimization on the sampled latent variables, and then reconstruct the latent variables in the high-dimensional physical property space for utilization. Accordingly, the computing system 1000 may automatically determine reasonable characteristic values for physical properties not input by the user, and may apply these values to predict and output a valid molecular structure that is valid and consistent with the physical-property characteristics provided by the user.

As a result, the computing system 1000 may maintain overall consistency among physical properties for molecular structure prediction and enhance the accuracy thereof even when user input is limited.

Referring back to FIG. 23, the computing system 1000 according to an embodiment of the present disclosure may also execute a deep learning prediction process using the obtained high-dimensional latent variables as target characteristics (S311).

Here, the target characteristics according to an embodiment may refer to an optimal solution to be achieved by output data according to a prediction process of a predetermined deep learning model.

In particular, in an embodiment, the target characteristics may refer to an optimal solution that the output data (e.g., molecular structural formula data, etc.) according to the prediction process of the multi-tasking learning model MtLM is intended to achieve.

In detail, in an embodiment, the computing system 1000 may provide the high-dimensional latent variables (T-dimensional population) obtained according to the above-described process to the multi-tasking learning model MtLM.

Then, the multi-tasking learning model MtLM may execute a prediction process using the provided high-dimensional latent variables (T-dimensional population) as target characteristics.

In this regard, in other words, the multi-tasking learning model MtLM according to an embodiment may execute a prediction process in which characteristic values (e.g., physical property input information) for respective N (1<=N<T) physical properties (i.e., N-dimensional physical properties) are used as input data, and predetermined molecular structural formula data satisfying the input N physical property values is used as output data.

Here, in other words, the molecular structural formula data may be data representing and storing structures such as compositions and binding methods of molecules, and may be data including molecular structural formulas satisfying N physical property values corresponding to physical property input information and representing physically valid combinations of physical properties in an embodiment.

In the above process, the multi-tasking learning model MtLM may execute the above prediction process in a direction that satisfies the given target characteristics.

In other words, the multi-tasking learning model MtLM may execute a prediction process of generating a molecular structural formula in a direction of reflecting the characteristics of the physical properties input by the user while appropriately combining the characteristics of the remaining physical properties not input by the user.

In this regard, in an embodiment, the multi-tasking learning model MtLM may execute the prediction process based on geometric alignment in the above-described integrated latent space M.

Accordingly, in an embodiment, the computing system 1000 may execute the deep learning prediction process using the high-dimensional latent variables as the target characteristics by interoperating with the multi-tasking learning model MtLM.

In addition, the computing system 1000 according to an embodiment of the present disclosure may provide output data according to the executed deep learning prediction process (S313).

In detail, in an embodiment, the multi-tasking learning model MtLM may execute the prediction process using the high-dimensional latent variables (T-dimensional population) as the target physical properties as described above, thereby generating molecular structural formula data reflecting the characteristics of the physical properties input by the user while plausibly combining the characteristics of the remaining physical properties not input by the user.

Then, the multi-tasking learning model MtLM may provide the generated molecular structural formula data to the computing system 1000.

Accordingly, in an embodiment, the computing system 1000 may obtain molecular structural formula data designed in a form valid for actual molecular structure formation while possessing the physical property characteristics desired by the user. In addition, in an embodiment, the computing system 1000 may provide the obtained molecular structural formula data in a predetermined manner.

In an embodiment, the computing system 1000 may provide the molecular structural formula data obtained as described above in association with a predetermined application service (e.g., a material synthesis/evaluation service, a material physical property prediction service, and/or an optimal material recommendation service).

As described above, in an embodiment of the present disclosure, the computing system 1000 may efficiently search for and optimize a high-dimensional space using a low-dimensional Gaussian space, thereby reducing the calculation cost of the universal multi-tasking learning model MtLM that handles large-scale data and directly improving performance thereof.

In particular, accordingly, the computing system 1000 may easily derive a set of T-dimensional physical properties required for output data generation of the multi-tasking learning model MtLM at low cost/high efficiency and provide the same with high accuracy.

In addition, accordingly, even when a user has a low understanding of physical properties other than those of interest, when simply receiving desired characteristics of physical properties, the computing system 1000 may generate and provide a molecular structure following a physically valid combination of physical properties while satisfying the desired characteristics, without having to separately infer unknown physical properties related to an unknown target material under development.

In addition, accordingly, even when data for specific physical properties are insufficient, the computing system 1000 may automatically assign reasonable values to the corresponding physical properties, thereby effectively improving the general applicability of the multi-tasking learning model MtLM that simultaneously processes various physical properties.

In addition, accordingly, even when new physical properties are added, the computing system 1000 may apply the same processes of sampling in the low-dimensional Gaussian space and restoring to the high-dimensional space, thereby also significantly improving the scalability of the multi-tasking learning model MtLM.

As described above, a method and a system for providing a guide for improving the performance of a multi-tasking model according to an embodiment of the present disclosure may efficiently search for and optimize data in a high-dimensional space through data sampling using a low-dimensional space, thereby reducing the calculation cost of a general-purpose multi-tasking model that handles large-scale data and directly improving the performance thereof.

In addition, accordingly, a method and a system for providing a guide for improving the performance of a multi-tasking model according to an embodiment of the present disclosure may automatically assign valid values for missing data during a data-dimensional restoration process, even when at least some of the data required for deep learning prediction are absent.

Accordingly, a method and a system for providing a guide for improving the performance of a multi-tasking model according to an embodiment of the present disclosure may naturally combine values automatically assigned to missing data with the given data even without a separate data request for the missing data, thereby generating and providing physically/logically reasonable outputs.

Therefore, a method and a system for providing a guide for improving the performance of a multi-tasking model according to an embodiment of the present disclosure may further expand the applicability range of a general-purpose multi-tasking model that processes a large amount of various data.

### [Method for Providing Guide Supporting Performance Enhancement of Multi-Tasking Model]

Hereinafter, a method by which the computing system 1000 according to an embodiment of the present disclosure provides a guide for supporting performance enhancement of a multi-tasking model (i.e., a method for implementing a multi-tasking model guide provision service) will be described in detail with reference to the accompanying drawings.

In the following description, descriptions overlapping the above-described contents may be summarized or omitted.

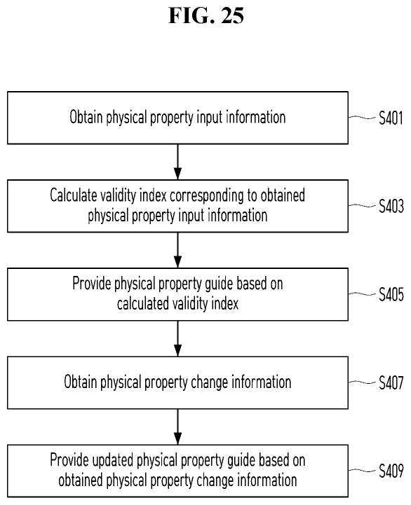

FIG. 25 illustrates a block flowchart for describing a method for providing a guide supporting performance enhancement of a multi-tasking model according to an embodiment of the present disclosure.

Specifically, referring to FIG. 25, a method for providing a guide supporting performance enhancement of a multi-tasking model according to an embodiment of the present disclosure may include obtaining predetermined physical property input information (S401), calculating a validity index corresponding to the obtained physical property input information (S403), providing a physical property guide based on the calculated validity index (S405), obtaining predetermined physical property change information (S407), and providing an updated physical property guide based on the obtained physical property change information (S409).

In detail, the computing system 1000 according to an embodiment of the present disclosure may obtain predetermined physical property input information (S401).

Here, in other words, the physical property input information according to an embodiment may correspond to information derived from user input of characteristic values (i.e., physical property values) for respective N physical properties (where 1≤N<T) (i.e., N-dimensional physical properties), thereby defining an N-dimensional physical-property space.

In detail, in an embodiment, the computing system 1000 may obtain physical property input information including N physical property values (i.e., target physical property values) based on user input through a predetermined user interface.

In particular, the computing system 1000 may obtain physical property input information specifying physical property values that the user intends to satisfy with respect to output data (e.g., molecular structural formula data, etc.) of the multi-tasking learning model MtLM.

In addition, the computing system 1000 according to an embodiment of the present disclosure may calculate a validity index corresponding to the obtained physical property input information (S403).

Here, the validity index according to an embodiment may refer to data obtained by quantitatively evaluating the difficulty in generation of output data (e.g., molecular structural formula data, etc.) generated based on predetermined input data (e.g., physical property input information, etc.).

In particular, in an embodiment, the validity index may be data that numerically represent actual feasibility (generation probability) of molecular structural formula data generated based on the predetermined physical property input information.

In other words, in an embodiment, the validity index may be data obtained by quantifying the possibility of generating the molecular structural formula as a result of executing the above-described steps S301, S302, S303, S304, S305, S306, S307, S308, S309, S310, S311, S312, and S313 based on given physical property input information (i.e., target physical property values).

The validity index according to an embodiment may be set in inverse proportion to the generation difficulty. In particular, in an embodiment, the validity index may decrease as the generation difficulty increases and may increase as the generation difficulty decreases.

In an embodiment, such a validity index may include first to third validity indices based on a predetermined density estimation algorithm, a fourth validity index based on a predetermined anomaly detection algorithm, and/or a fifth validity index based on a similarity determination algorithm.

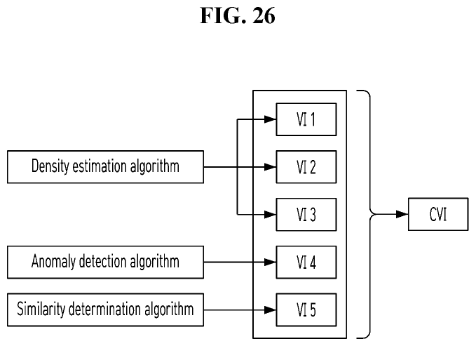

FIG. 26 illustrates an example of a diagram for describing a validity index according to an embodiment of the present disclosure.

In detail, referring to FIG. 26, in an embodiment, the computing system 1000 may calculate a validity index for the obtained physical property input information using various deep learning algorithms.

In this regard, in an embodiment, the computing system 1000 may calculate validity index for the physical property input information using at least some of the various deep learning algorithms to be described below.

In addition, the various deep learning algorithms used in an embodiment may operate by being directly included in the multi-tasking learning model MtLM or may operate by being implemented separately from the multi-tasking learning model MtLM.

In more detail, in an embodiment, the computing system 1000 may calculate 1) a validity index based on a density estimation algorithm.

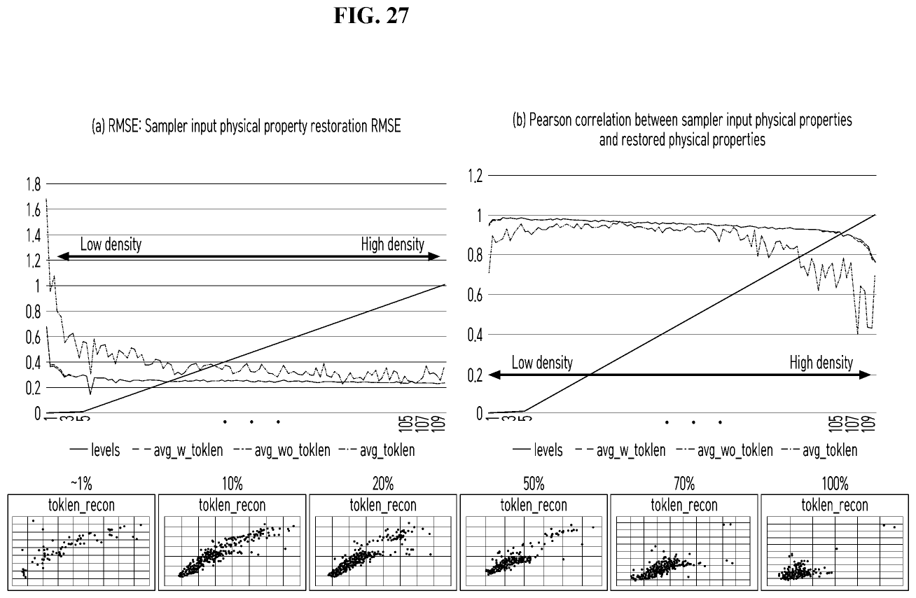

FIG. 27 illustrates an example of a diagram for describing a background of utilization of a density estimation algorithm according to an embodiment of the present disclosure.

(a) in FIG. 27 illustrates a graph visualizing a root mean square error (RMSE) relationship generated in a process in which the sampler module SPM restores given physical property input information according to an embodiment of the present disclosure.

Here, the x-axis of the graph of (a) in FIG. 27 represents data density (with higher density toward the right), and the y-axis represents an RMSE value.

Referring to (a) in FIG. 27, it may be confirmed that the RMSE values tend to be high in low-density regions. This may mean that it is difficult to execute accurate restoration in regions where an amount of training data is small or unevenly distributed.

Conversely, it may be confirmed that, in high-density regions, the RMSE values tend to be low, and restoration tends to be executed more accurately compared to the low-density regions. This may mean that the restoration performance is enhanced in regions where an amount of training data is sufficient.

In particular, it may be seen in (a) in FIG. 27 that the restoration performance of the model is degraded in regions where the density of training data is low, whereas the restoration performance of the model is enhanced in regions where the density of training data is high.

(b) in FIG. 27 illustrates a graph visualizing a Pearson correlation coefficient relationship between input data (i.e., physical property input information) and restored data (i.e., high-dimensional latent variables) of the sampler module SPM according to an embodiment of the present disclosure.

Here, the x-axis of the graph of (b) in FIG. 27 represents data density (with higher density toward the right), and the y-axis represents a Pearson correlation coefficient value.

Referring to (b) in FIG. 27, it may be confirmed that the Pearson correlation coefficient values tend to be low in low-density regions. This may mean that correlations between the input physical properties and the restored physical properties are low in regions where an amount of training data is insufficient.

Conversely, it may be confirmed that, in high-density regions, the Pearson correlation coefficient values tend to be high, and the correlations between the input physical properties and the restored physical properties tend to increase. This may mean that the model has been well trained in regions where sufficient amount of training data exists.

In particular, it may be seen in (b) in FIG. 27 that the prediction accuracy of the model is degraded in regions where the density of training data is low, whereas the prediction performance of the model is enhanced (i.e., the input physical properties are restored with higher accuracy) in regions where the density of training data is high.

As shown in (a) and (b) in FIG. 27, it may be confirmed that the lower the data density, the higher the difficulty of generating output data (e.g., molecular structural formula data, etc.), and it may be confirmed that there exists a correlation between the density of the input physical properties (i.e., low-dimensional data) and the density of restored physical properties (i.e., high-dimensional data).

Based on the above, the computing system 1000 according to an embodiment of the present disclosure intends to calculate a validity index for given data in consideration of the density of the given data (e.g., a degree of input of physical properties).

Specifically, in an embodiment, the computing system 1000 may 1] calculate a first validity index VI 1, which is a validity index based on the physical property input information, based on a density estimation algorithm.

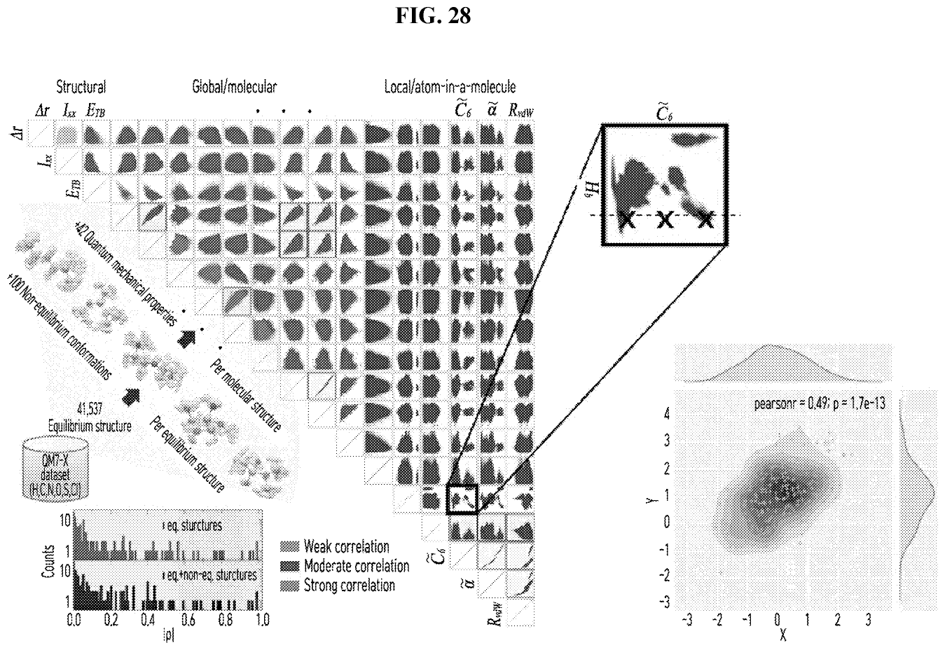

FIG. 28 illustrates examples of output data based on a density estimation algorithm according to an embodiment of the present disclosure.

In detail, referring to FIG. 28, in an embodiment, the computing system 1000 may obtain a density value (hereinafter, a first input data density) estimated for physical property input information by interoperating with the generation difficulty evaluation module GDM trained as described above.

In more detail, in an embodiment, the computing system 1000 may input the physical property input information to the generation difficulty evaluation module GDM.

Then, the generation difficulty evaluation module GDM may estimate a density value for the input physical property input information based on pre-learned information and provide the estimated density value to the computing system 1000.

Accordingly, in an embodiment, the computing system 1000 may obtain the density value (i.e., the first input data density) for the physical property input information from the generation difficulty evaluation module GDM.

In addition, in an embodiment, the computing system 1000 may calculate the above-described first validity index VI 1 based on the obtained first input data density.

In an embodiment, the computing system 1000 may calculate the first validity index VI 1 in proportion to the first input data density.

In particular, in an embodiment, the computing system 1000 may calculate the first validity index VI 1 such that the first validity index VI 1 increases as the first input data density increases and decreases as the first input data density decreases.

In another embodiment, the computing system 1000 may set the first validity index VI 1 through comparison between the first input data density and a predetermined reference value.

For example, the computing system 1000 may set the first validity index VI 1 to “high” when the first input data density is equal to or greater than the predetermined reference value, and may set the first validity index VI 1 to “low” when the first input data density is less than the predetermined reference value.

In addition, in an embodiment, the computing system 1000 may 2] calculate a second validity index VI 2, which is a validity index based on low-dimensional latent variables, based on a density estimation algorithm.

In detail, in an embodiment, the computing system 1000 may obtain low-dimensional latent variables corresponding to the obtained physical property input information.

Here, in other words, the low-dimensional latent variables according to an embodiment may refer to data obtained by projecting and transforming given input data into a low-dimensional Gaussian space. In an embodiment, such low-dimensional latent variables may be represented as respective points existing in a low-dimensional Gaussian space.

In detail, in an embodiment, the computing system 1000 may obtain low-dimensional latent variables based on the physical property input information by applying the description disclosed in step S303 above.

In particular, the computing system 1000 may obtain the low-dimensional latent variables corresponding to the physical property input information by projecting the physical property input information into a low-dimensional Gaussian space and converting the same to be represented in an L-dimensional space (where 1≤L<N, T) through interoperation with the sampler module SPM.

In addition, in an embodiment, the computing system 1000 may obtain a density value (hereinafter, a second input data density) estimated for the low-dimensional latent variables obtained as described above by interoperating with the generation difficulty evaluation module GDM.

In detail, in an embodiment, the computing system 1000 may input the low-dimensional latent variables to the generation difficulty evaluation module GDM.

Then, the generation difficulty evaluation module GDM may estimate the density value for the input low-dimensional latent variables based on pre-learned information and provide the estimated density value to the computing system 1000.

Accordingly, in an embodiment, the computing system 1000 may obtain the density value (i.e., the second input data density) for the low-dimensional latent variables from the generation difficulty evaluation module GDM.

In addition, in an embodiment, the computing system 1000 may calculate the above-described second validity index VI 2 based on the obtained second input data density.

In an embodiment, the computing system 1000 may calculate the second validity index VI 2 in proportion to the second input data density.

In particular, in an embodiment, the computing system 1000 may calculate the second validity index VI 2 such that the second validity index VI 2 increases as the second input data density increases and decreases as the second input data density decreases.

In another embodiment, the computing system 1000 may set the second validity index VI 2 through comparison between the second input data density and a predetermined reference value.

For example, the computing system 1000 may set the second validity index VI 2 to “high” when the second input data density is equal to or greater than the predetermined reference value, and may set the second validity index VI 2 to “low” when the second input data density is less than the predetermined reference value.

In addition, in an embodiment, the computing system 1000 may 3] calculate a third validity index VI 3, which is a validity index based on high-dimensional latent variables, based on a density estimation algorithm.

Here, in other words, the high-dimensional latent variables according to an embodiment may refer to data obtained by sampling given input data in a low-dimensional Gaussian space and then restoring the sampled input data into a high-dimensional space again through an optimization process.

In an embodiment, such high-dimensional latent variables may form physically valid combinations of physical properties while satisfying N physical property values (i.e., target physical property values) desired by the user.

In detail, the computing system 1000 according to an embodiment of the present disclosure may obtain high-dimensional latent variables based on the physical property input information by applying the processes disclosed in steps S305 to S309 above.

Specifically, in an embodiment, the computing system 1000 may obtain sampled latent variables based on the low-dimensional latent variables obtained as described above (S305). In addition, in an embodiment, the computing system 1000 may obtain optimized latent variables from the sampled latent variables (S307). In addition, in an embodiment, the computing system 1000 may obtain high-dimensional latent variables based on the optimized latent variables (S309). A detailed description thereof will be omitted, as the procedures of steps S305, S306, S307, S308, and S309 apply analogously.

In addition, in an embodiment, the computing system 1000 may obtain a density value (hereinafter, a third input data density) estimated for the high-dimensional latent variables obtained as described above by interoperating with the generation difficulty evaluation module GDM.

In detail, in an embodiment, the computing system 1000 may input the high-dimensional latent variables to the generation difficulty evaluation module GDM.

Then, the generation difficulty evaluation module GDM may estimate a density value for the input high-dimensional latent variables based on pre-learned information and provide the estimated density value to the computing system 1000.

Accordingly, in an embodiment, the computing system 1000 may obtain a density value (i.e., a third input data density) for the high-dimensional latent variables from the generation difficulty evaluation module GDM.

In addition, in an embodiment, the computing system 1000 may calculate the above-described third validity index VI 3 based on the obtained third input data density.

In an embodiment, the computing system 1000 may calculate the third validity index VI 3 in proportion to the third input data density.

In particular, in an embodiment, the computing system 1000 may calculate the third validity index VI 3 such that the third validity index VI 3 increases as the third input data density increases and 3 decreases as the third input data density decreases.

In another embodiment, the computing system 1000 may set the first validity index VI 1 through comparison between the third input data density and a predetermined reference value.

For example, the computing system 1000 may set the third validity index VI 3 to “high” when the third input data density is equal to or greater than the predetermined reference value, and may set the third validity index VI 3 to “low” when the third input data density is less than the predetermined reference value.

As described above, in an embodiment, the computing system 1000 may analyze the density of given data and determine the difficulty of generating a molecular structural formula generated based on the given data.

Accordingly, the computing system 1000 may determine and provide a generation probability for a molecular structural formula corresponding to given data (here, physical property input information) based on a reasonable determination grounded in the objective observation that a correlation exists between the density of input physical properties (i.e., low-dimensional data) and the density of restored physical properties (i.e., high-dimensional data). In particular, the computing system 1000 may determine the generation probability in consideration of the fact that, as the density of the data decreases, the difficulty associated with generating corresponding output data (e.g., molecular structural formula data) increases.

In this regard, in an embodiment, the computing system 1000 may analyze the density of the given data in various dimensions in a multifaceted manner, thereby further improving the accuracy and reliability of the calculated validity index.

In this regard, according to an embodiment, the computing system 1000 may calculate the third validity index VI 3 by further utilizing a marginal probability distribution estimation algorithm.

Here, for reference, the marginal probability distribution estimation algorithm may be an algorithm for calculating a probability distribution of specific variables or a set of variables in a multi-dimensional probability distribution, and may be an algorithm for calculating a marginal probability of specific variables and analyzing influences of the remaining variables in an integrated manner.

In detail, in an embodiment, the computing system 1000 may calculate a marginal probability based on high-dimensional latent variables using the marginal probability distribution estimation algorithm.

In an embodiment, when evaluating a possibility of a first physical property in T-dimensional (e.g., 45-dimensional) latent variables, the computing system 1000 may calculate a marginal probability by executing integration on the remaining (T−1) variables.

In addition, the computing system 1000 may calculate the above-described third validity index VI 3 by reflecting the calculated marginal probability.

In particular, according to an embodiment, the computing system 1000 may analyze a combined distribution with respect to specific variables in high-dimensional data (here, a high-dimensional latent variable) using the marginal probability distribution estimation algorithm, and calculate the corresponding third validity index VI 3 based thereon.

Accordingly, the computing system 1000 may implement more efficient data processing for high-dimensional data, for which it is difficult to evaluate combined distributions of all variables, and may easily calculate a corresponding validity index (here, the third validity index VI 3).

In one example, according to embodiments, the computing system 1000 may evaluate the performance of the generation difficulty evaluation module GDM based on the second input data density (i.e., the density value estimated for the low-dimensional latent variables) and the third input data density (i.e., the density value estimated for the high-dimensional latent variables).

In detail, in an embodiment, the computing system 1000 may determine mutual similarity through comparison between the second input data density (i.e., low-dimensional latent variable density) and the third input data density (i.e., high-dimensional latent variable density).

In more detail, in an embodiment, the computing system 1000 may identify a region having a low density (hereinafter, a low-density region) and a region having a high density (hereinafter, a high-density region) in each of the second input data density and the third input data density.

In addition, the computing system 1000 may compare a data distribution according to the second input data density with a data distribution according to the third input data density within each of the identified low-density region and high-density region.

Accordingly, in an embodiment, the computing system 1000 may determine mutual similarity between the second input data density and the third input data density.

In addition, in an embodiment, the computing system 1000 may determine a correlation between the second input data density and the third input data density in proportion to the determined similarity.

In particular, the computing system 1000 may determine that the correlation between the data distribution according to the second input data density and the data distribution according to the third input data density is higher as the determined similarity increases.

Accordingly, in an embodiment, the computing system 1000 may verify whether the second input data density (i.e., the low-dimensional latent variable density) is valid as a predictive index of the third input data density (i.e., the high-dimensional latent variable density), and may simultaneously evaluate the estimated performance of the generation difficulty evaluation module GDM.

Continuously, in an embodiment, the computing system 1000 may train the generation difficulty evaluation module GDM to execute a density value estimation process in a direction in which the above-described correlation increases (i.e., a direction in which the second input data density and the third input data density become more similar).

Accordingly, the computing system 1000 may further enhance the accuracy and reliability of the generation difficulty evaluation module GDM.

In one example, according to embodiments, the computing system 1000 may calculate a validity index (not shown; hereinafter, an intermediate-dimensional validity index) based on latent variables (hereinafter, intermediate-dimensional latent variables) defined in an M-dimensional space (where L<M<T) that exists between an L-dimensional low-dimensional Gaussian space and a T-dimensional high-dimensional space. The intermediate-dimensional validity index may be calculated using a density estimation algorithm.

In detail, in an embodiment, the computing system 1000 may obtain the intermediate-dimensional latent variables, which are data obtained by restoring the optimized latent variables based on the low-dimensional Gaussian space as described above into a predetermined M-dimensional space.

In this regard, a specific method by which the computing system 1000 according to an embodiment restores L-dimensional latent variables into M-dimensional latent variables will be omitted by applying the description of the method for restoring the L-dimensional latent variables into the T-dimensional latent variables above.

In addition, in an embodiment, the computing system 1000 may obtain a density value (hereinafter, an intermediate-dimensional input data density) estimated for the intermediate-dimensional latent variables obtained as described above by interworking with the generation difficulty evaluation module GDM.

In more detail, in an embodiment, the computing system 1000 may input the intermediate-dimensional latent variables to the generation difficulty evaluation module GDM.

Then, the generation difficulty evaluation module GDM may estimate a density value of the input intermediate-dimensional latent variables based on pre-learned information and provide the estimated density value to the computing system 1000.

Accordingly, in an embodiment, the computing system 1000 may obtain the density value (i.e., the intermediate-dimensional input data density) for the intermediate-dimensional latent variables from the generation difficulty evaluation module GDM.

In addition, in an embodiment, the computing system 1000 may calculate the above-described intermediate-dimensional validity index based on the obtained intermediate-dimensional input data density.

In an embodiment, the computing system 1000 may calculate the intermediate-dimensional validity index in proportion to the intermediate-dimensional input data density.

In particular, in an embodiment, the computing system 1000 may calculate the intermediate-dimensional validity index such that the higher the intermediate-dimensional input data density results in a higher the intermediate-dimensional validity index, and the lower the intermediate-dimensional input data density results in a lower the intermediate-dimensional validity index.

In another embodiment, the computing system 1000 may set the intermediate-dimensional validity index through comparison between the intermediate-dimensional input data density and a predetermined reference value.

For example, the computing system 1000 may set the intermediate-dimensional validity index to “high” when the intermediate-dimensional input data density is equal to or greater than the predetermined reference value, and may set the intermediate-dimensional validity index to “low” when the intermediate-dimensional input data density is less than the predetermined reference value.

Accordingly, in an embodiment, the computing system 1000 may easily avoid situations in which the accuracy of density estimation is deteriorated due to data sparsity in a low-dimensional space and/or a high-dimensional space.

Referring back to FIG. 26, in addition, in an embodiment, the computing system 1000 may 2) calculate a validity index based on an anomaly detection algorithm.

In detail, in an embodiment, the computing system 1000 may obtain a fourth validity index VI 4, which is a validity index calculated using a predetermined anomaly detection algorithm.

Here, for reference, the anomaly detection algorithm may be an algorithm for detecting outliers different from normal patterns in a predetermined data set.

In an embodiment, the computing system 1000 may detect outliers based on at least one of the above-described first to third input data densities using the above-described anomaly detection algorithm, and calculate the above-described fourth validity index VI 4 according to the number of detected outliers.

The outliers detected from at least one of the first to third input data densities may refer to data points (i.e., outliers) spaced apart from most data points (i.e., data points of normal patterns) among a plurality of data points included in the specific input data density.

In other words, the outliers may indicate the existence of data points spaced apart from other data points, which may assume a low density among data points.

Accordingly, in an embodiment, the computing system 1000 may determine that the data density decreases as the number of outliers increases, and accordingly, may set a lower validity index (i.e., a higher generation difficulty).

In more detail, in an embodiment, the computing system 1000 may execute anomaly detection based on at least one of the first to third input data densities using a predetermined anomaly detection algorithm (e.g., K-nearest neighbors (KNN) density estimation and/or local outlier factor (LOF)).

Accordingly, the computing system 1000 may detect outliers based on at least one of the first to third input data densities.

In an embodiment, the computing system 1000 may detect at least one data point (i.e., a data point in a low-density region) having an average distance from other data points equal to or greater than a predetermined reference value from at least one of the first to third input data densities.

Then, the computing system 1000 may identify the detected at least one data point as an outlier.

Further, in an embodiment, the computing system 1000 may count the number of identified outliers.

In addition, in an embodiment, the computing system 1000 may calculate the fourth validity index VI 4 in inverse proportion to the number of outliers counted.

In particular, the computing system 1000 may calculate the fourth validity index VI 4 such that the fourth validity index VI 4 decreases as the number of outliers (i.e., the number of data points existing in the low-density regions) increases, and the fourth validity index VI 4 increases as the number of outliers decreases.

In another embodiment, the computing system 1000 may set the fourth validity index VI 4 by comparing the number of outliers with a predetermined reference value.

For example, the computing system 1000 may set the fourth validity index VI 4 to “high” when the number of outliers is equal to or greater than the predetermined reference value, and may set the fourth validity index VI 4 to “low” when the number of outliers is less than the predetermined reference value.

As described above, in an embodiment, the computing system 1000 may detect outliers within given data through anomaly detection, and may identify the density of the corresponding data according to the number of detected outliers to calculate a validity index. In this regard, in an embodiment, the computing system 1000 may contribute to adjusting abnormal distributions of data and increasing the prediction accuracy of the model by utilizing the anomaly detection.

Further referring to FIG. 26, in an embodiment, the computing system 1000 may 3) calculate a validity index based on a similarity determination algorithm.

In detail, in an embodiment, the computing system 1000 may obtain a fifth validity index VI 5, which is a validity index calculated using a predetermined similarity determination algorithm.

Here, for reference, a similarity determination algorithm may be an algorithm that measures similarity between predetermined data and represents the similarity as a numerical value.

In an embodiment, the computing system 1000 may measure similarity between output data (i.e., molecular structural formula data, etc.) according to an embodiment of the present disclosure and actual data (i.e., actual molecular structural formula data, etc.) using the above-described similarity determination algorithm, and obtain data (hereinafter, a similarity numerical value) quantifying the measured similarity.

The similarity numerical value according to an embodiment may be an index indicating how similar the output data based on given data (here, physical property input information) is to the actual data.

Accordingly, in an embodiment, the computing system 1000 may determine that data density increases as the similarity numerical value increases, and accordingly, may set a higher validity index (i.e., a lower generation difficulty).

In more detail, in an embodiment, the computing system 1000 may generate output data corresponding to the high-dimensional latent variables obtained as described above.

In an embodiment, the computing system 1000 may generate output data (e.g., molecular structural formula data, etc.) corresponding to high-dimensional latent variables by applying the processes disclosed in steps S311 to S313 above.

Specifically, in an embodiment, the computing system 1000 may execute a deep learning prediction process using the obtained high-dimensional latent variables as target characteristics (S311). In addition, in an embodiment, the computing system 1000 may generate output data according to the executed deep learning prediction process (S313). A detailed description thereof will be omitted by applying the description in steps S311 to S313.

Accordingly, in an embodiment, the computing system 1000 may generate molecular structural formula data in which characteristics of physical properties input by a user are reflected while characteristics of the remaining physical properties not input by the user are validly combined by executing a prediction process using high-dimensional latent variables as target physical properties.

In particular, in an embodiment, the computing system 1000 may generate molecular structural formula data designed in a form valid for actual molecular structure formation while retaining physical property-specific characteristics desired by the user.

Continuously, in an embodiment, the computing system 1000 may obtain a similarity numerical value according to the generated output data (i.e., molecular structural formula data, etc.) using a predetermined similarity determination algorithm (e.g., a generative adversarial network (GAN), a Euclidean distance, a Cosine similarity, a Jaccard similarity, a Pearson correlation coefficient, and/or a cross-entropy, etc.).

In the following embodiment, the similarity determination algorithm will be described based on the generative adversarial network (GAN), but the present disclosure is not limited thereto.

Here, for reference, the generative adversarial network may include a discriminator model that determines whether input data corresponds to pre-trained real data or newly generated data and outputs a probability score corresponding thereto.

In this regard, in order for the discriminator model to operate as described above, the generative adversarial network may be pre-trained based on a training data set including a plurality of molecular structural formula data (i.e., actual data).

In detail, in an embodiment, the computing system 1000 may input the generated output data to the generative adversarial network.

Then, the generative adversarial network may output a probability score for the input output data using a discriminator model included in the generative adversarial network.

In particular, the generative adversarial network may output a probability score, which is data obtained by quantifying a probability that the output data (e.g., molecular structural formula data, etc.) generated according to an embodiment of the present disclosure corresponds to pre-trained actual molecular structural formula data (i.e., label).

Then, the generative adversarial network may provide the output probability score to the computing system 1000.

Accordingly, in an embodiment, the computing system 1000 may obtain a probability score indicating a probability that the output data corresponds to actual data (i.e., a degree to which the output data is similar to actual data) via the generative adversarial network.

In addition, in an embodiment, the computing system 1000 may calculate a similarity numerical value based on the probability score obtained as described above.

In addition, in an embodiment, the computing system 1000 may calculate the fifth validity index VI 5 in proportion to the calculated similarity numerical value.

In particular, in an embodiment, the computing system 1000 may evaluate that the combination of physical properties according to the output data is more similar to the combination of physical properties according to the actual data as the similarity numerical value (probability score) increases, and calculate the fifth validity index VI 5 reflecting this evaluation.

In an embodiment, the computing system 1000 may calculate the fifth validity index VI 5 such that the fifth validity index VI 5 increases as the similarity numerical value increases and decreases as the similarity numerical value decreases.

In another embodiment, the computing system 1000 may set the fifth validity index VI 5 through comparison between the similarity numerical value and a predetermined reference value.

For example, the computing system 1000 may set the fifth validity index VI 5 to “high” when the similarity numerical value is equal to or greater than the predetermined reference value, and may set the fifth validity index VI 5 to “low” when the similarity numerical value is less than the predetermined reference value.

As described above, in an embodiment, the computing system 1000 may calculate a validity index (i.e., an actual generation possibility) for given physical property input information by determining similarity between the output data according to the given physical property input information and the actual data.

Referring back to FIG. 25, as described above, in an embodiment, the computing system 1000 may calculate the first to fifth validity indices VI 1, VI 2, VI 3, VI 4, and VI 5 indicating validity (i.e., generation difficulty, generation probability, and/or feasibility) of physical property input information using various deep learning algorithms.

In other words, the computing system 1000 may analyze validity by determining how high a probability of success is in a molecular generation process corresponding to physical property values input by a user and how similar the physical property values input by the user are to the actual data, and may calculate a validity index that quantitatively represent the analyzed validity.

As such, in an embodiment, the computing system 1000 may calculate and provide a validity index that quantitatively represents the molecular generation difficulty corresponding to the physical property values input by the user.

Accordingly, the computing system 1000 may easily provide a guide index supporting molecular generation that is physically/scientifically feasible while following physical property values input by the user, based on objective grounds.

In this regard, in an embodiment, the computing system 1000 may determine the above validity index using various deep learning algorithms, thereby further improving the accuracy and reliability of the validity index.

In addition, the computing system 1000 according to an embodiment of the present disclosure may provide a physical property guide based on the calculated validity index (S405).

Here, the physical property guide according to an embodiment may refer to data presenting physical properties and/or physical property values that enhance physical/scientific validity of a molecular structural formula (i.e., a combination of physical properties) generated according to predetermined physical property input information.

In particular, the physical property guide according to an embodiment may refer to data that evaluates whether a molecular structural formula following physical property values input by a user is physically/scientifically valid, and then guides a change (modification) direction for specific physical properties and/or physical property values to enhance validity based on the evaluation result.

According to an embodiment, such a physical property guide may include analysis information (e.g., analysis data according to the first to fifth validity indices VI 1, VI 2, VI 3, VI 4, and VI 5) indicating causes for deriving the presented change (modification) direction.

In addition, the physical property guide according to an embodiment may further refer to data presenting a learning method for enhancing physical/scientific validity of a molecular structural formula (i.e., a combination of physical properties) generated according to predetermined physical property input information.

In an embodiment, the physical property guide may include data presenting a method for requesting additional data for regions having low validity or supplementing training data.

In detail, in an embodiment, the computing system 1000 may generate the above-described physical property guide based on the first to fifth validity indices VI 1, VI 2, VI 3, VI 4, and VI 5 calculated as described above.

In more detail, referring to FIG. 26, in an embodiment, the computing system 1000 may obtain a comprehensive validity index CVI based on the first to fifth validity indices VI 1, VI 2, VI 3, VI 4, and VI 5.

Here, the comprehensive validity index CVI according to an embodiment may refer to data obtained by integrating (aggregating) at least some of the first to fifth feasibility indices VI 1, VI 2, VI 3, VI 4, and VI 5 into a single value according to a predetermined method.

In particular, in an embodiment, the computing system 1000 may obtain the comprehensive validity index CVI by integrating the first to fifth validity indices VI 1, VI 2, VI 3, VI 4, and VI 5 into one data based on a predetermined method (e.g., average value calculation, etc.).

In this regard, according to an embodiment, the computing system 1000 may preset importance (weights) for the respective first to fifth validity indices VI 1, VI 2, VI 3, VI 4, and VI 5 according to user settings.

In addition, the computing system 1000 may obtain the comprehensive validity index CVI in which the first to fifth validity indices VI 1, VI 2, VI 3, VI 4, and VI 5 are integrated into one according to a predetermined method by reflecting the preset importance (weights).

In addition, in an embodiment, the computing system 1000 may generate the above-described physical property guide based on the obtained comprehensive validity index CVI.

In detail, in an embodiment, the computing system 1000 may generate a physical property recommendation guide and/or a physical property value recommendation guide based on the comprehensive validity index CVI.

Here, the physical property recommendation guide according to an embodiment may refer to a physical property guide that proposes a physical property change that increases the comprehensive validity index CVI based on predetermined property input information.

In particular, in an embodiment, the physical property recommendation guide may be data proposing physical properties to be removed, replaced, and/or added to increase the comprehensive validity index CVI among physical properties related to predetermined physical property input information.

In addition, the physical property value recommendation guide according to an embodiment may refer to a physical property guide configured to propose changes to physical property values (where, the values include ranges) that increase the comprehensive validity index CVI based on predetermined physical property input information.

In particular, in an embodiment, the physical property value recommendation guide may be data proposing physical property values to be updated to increase the comprehensive validity index CVI among characteristic values (i.e., physical property values) of physical properties associated with predetermined property input information.

In the above description, the physical property recommendation guide and the physical property value recommendation guide have been separately described for the sake of effective description, but various embodiments are possible, such as at least some of the above-described configurations may be operated in an organically combined manner according to embodiments.

In an embodiment, the physical property recommendation guide and the physical property value recommendation guide may be organically combined to implement a physical property guide configured to propose physical properties to be added to increase the comprehensive validity index CVI among physical properties associated with predetermined physical property input information, and characteristic values (i.e., physical property values) of the added physical properties.

Returning to the description, in particular, in an embodiment, the computing system 1000 may generate a physical property recommendation guide and/or a physical property value recommendation guide that proposes changes to predetermined physical properties and/or physical property values in a direction in which the comprehensive validity index CVI is increased (i.e., a direction that reduces generation difficulty).

Accordingly, in an embodiment, the computing system 1000 may generate a physical property guide that presents physical properties and/or physical property values that enhance physical/scientific validity of a molecular structural formula (i.e., a combination of physical properties) generated according to physical property input information input by a user.

For example, the computing system 1000 may generate a first physical property guide including the following data.

### [First Physical Property Guide: Removal and Replacement of Specific Physical Property Value]

Input physical property values: Physical property A: 35, physical property B: 25, physical property C: 80

Evaluation result: Based on isolation forest result, it is determined that molecular generation difficulty is very high because physical property C value 80 is abnormally high.

Proposed modification direction: It is proposed to use physical property D, which has high correlation with physical property C, instead of physical property C. It is proposed to set physical property D within a range from 40 to 50.

Modified physical property values: Physical property A: 35, physical property B: 25, physical property D: 45 (replacing physical property C, selected within the proposed range)

As another example, the computing system 1000 may generate a second physical property guide including the following data.

### [Second Physical Property Guide: Adjustment of Specific Physical Property Value Range]

Input physical property values: Physical property A: 30, physical property B: 15, physical property C: 45

Evaluation result: Based on KDE and GAN discriminator outputs, it is determined that molecular generation difficulty is high because density of physical property A is very low within the input range. It is confirmed that molecular generation is difficult in low-density regions.

Proposed modification direction: It is proposed to adjust the value of physical property A from 20 to 25. In this range, it is determined that molecular generation difficulty will be lowered due to high data density.

Modified physical property values: Physical property A: 22 (selected within the proposed range), physical property B: 15, physical property C: 45

As another example, the computing system 1000 may generate a third physical property guide including the following data.

### [Third Physical Property Guide: Simultaneous Adjustment of Multiple Physical Property Values]

Input physical property values: Physical property A: 10, physical property B: 50, physical property C: 70

Evaluation result: Based on LOF and autoencoder results, it is determined that the combination of physical property B and physical property C is abnormal. When physical property B and physical property C coexist, data density is low, and thus molecular generation difficulty is determined to be high.

Proposed modification direction: Considering the correlation between physical property B and physical property C, it is proposed to adjust the value of physical property B from 40 to 45 and to adjust the value of physical property C from 60 to 65. In these ranges, it is determined that the correlation between the two physical property values is further increased, and molecular generation difficulty is expected to be reduced.

Modified physical property values: Physical property A: 10, physical property B: 42 (selected within the proposed range), physical property C: 63 (selected within proposed range)

As described above, in an embodiment, the computing system 1000 may verify the validity of the molecular structural formula according to the physical property values input by the user, and may specifically present directions for changing physical properties and/or physical property values that increase the probability of success in molecular generation and guarantee physical/scientific validity simultaneously based thereon.

Accordingly, the computing system 1000 may support the multi-tasking model according to an embodiment to generate physically feasible molecules, may identify abnormal input values to prevent errors associated therewith, and accordingly, may enhance a molecular generation success rate of the multi-tasking model and continuously enhance performance thereof.

In addition, accordingly, the computing system 1000 may provide a user with a clear modification direction for molecular generation that guarantees physical/scientific validity, thereby also improving user experience and satisfaction.

In one example, according to an embodiment, the computing system 1000 may generate a learning recommendation guide based on the comprehensive validity index CVI.

Here, the learning recommendation guide according to an embodiment may refer to a physical property guide that presents a learning method for increasing the comprehensive validity index CVI based on predetermined physical property input information.

In particular, the computing system 1000 may generate a physical property guide including the above-described learning recommendation guide.

For example, the computing system 1000 may generate a fourth physical property guide including the following data.

### [Fourth Physical Property Guide: Insufficient Data of Specific Physical Property Value]

Input physical property values: Physical property A: 10, physical property B: 35, physical property C: 60

Evaluation result: Based on KDE and LOF, it is confirmed that the value 60 of physical property C has very low data density and that the model does not operate well at that value. Due to insufficient training data for physical property C, prediction performance of the model is degraded.

Proposed modification direction: It is proposed to supplement model training by collecting additional data in which the value of physical property C ranges from 55 to 65. When collecting new data, it is recommended to include various combinations in consideration of the values of physical properties A and B.

Additional data collection: Additional collection of new data points including physical property A: 10, physical property B: 35, physical property C: 55 to 65.

As another example, the computing system 1000 may generate a fifth physical property guide including the following data.

### [Fifth Physical Property Guide: Lack of High-Dimensional Correlation]

Input physical property values: Physical property A: 20, physical property B: 50, physical property C: 70

Evaluation result: Based on GAN discriminator outputs, it is confirmed that the combination of physical property B and physical property C is different from actual data and that the model has not sufficiently learned that combination. There is insufficient training data for the correlation between physical property B and physical property C.

Proposed modification direction: It is proposed to supplement learning by collecting additional data in which the value of physical property B ranges from 45 to 55 and the value of physical property C ranges from 65 to 75. It is also proposed to adjust the value of physical property A over wide ranges to increase data diversity.

Additional data collection: Additional collection of new data points including physical property A: various values, physical property B: 45 to 55, physical property C: 65 to 75.

As another example, the computing system 1000 may generate a sixth physical property guide including the following data.

### [Sixth Physical Property Guide: Degradation of Model Performance in Specific Region]

Input physical property values: Physical property A: 25, physical property B: 30, physical property C: 50

Evaluation result: Based on isolation forest result, it is identified that the combination of the value of 25 of physical property A and the value of 30 of physical property B is abnormal, and it is confirmed that performance of the model is degraded for the corresponding combination. Lack of training data for the corresponding combination results in poor prediction performance of the model.

Proposed modification direction: It is proposed to supplement model training by collecting additional data in which the value of physical property A ranges from 20 to 30 and the value of physical property B ranges from 25 to 35. It is further proposed to adjust the value of physical property C over wide ranges to increase data diversity.

Additional data collection: Additional collection of new data points including physical property A: 20 to 30, physical property B: 25 to 35, physical property C: various values.

As such, in an embodiment, the computing system 1000 may provide a detailed guide when additional learning is required for physical property values input by the user, thereby directly improving the performance of the corresponding multi-tasking model and consequently significantly improving the quality of output data (i.e., molecular structural formula data) of the model.

In this regard, according to embodiments, the computing system 1000 may generate and provide the above-described physical property guide when the above-described comprehensive validity index CVI is equal to or less than a predetermined reference value (i.e., when molecular generation difficulty corresponding to the physical property values input by the user is equal to or greater than the predetermined reference value).

Accordingly, the computing system 1000 may minimize load caused by indiscriminate data processing.

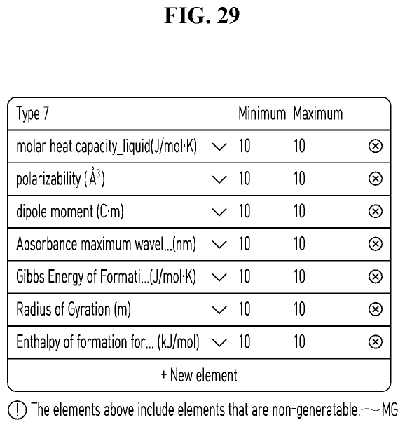

FIG. 29 illustrates an example of visualization of a physical property guide according to an embodiment of the present disclosure.

In addition, referring to FIG. 29, in an embodiment, the computing system 1000 may visualize and provide the physical property guide MG generated as described above in a predetermined manner.

In an embodiment, the computing system 1000 may visualize and provide the physical property guide MG based on a predetermined graphic image in which the physical property guide MG is displayed in correspondence with associated physical property input information and/or output data (i.e., molecular structural formula data, etc.).

In another embodiment, the computing system 1000 may visualize and provide the physical property guide MG based on a predetermined pop-up window displaying the physical property guide MG.

As such, the computing system 1000 may visually provide a quantitative evaluation of the validity of physical property values input by a user and/or a guide based thereon using various user interfaces.

Accordingly, the computing system 1000 may effectively support a user in more easily and intuitively understanding and recognizing the physical property guide MG.

In addition, the computing system 1000 according to an embodiment of the present disclosure may obtain predetermined physical property change information (S407).

Here, the physical property change information according to an embodiment may refer to information including changes in predetermined physical properties and/or physical property values according to user input.

In other words, the physical property change information may be information specifying physical properties and/or physical property values changed in response to user input.

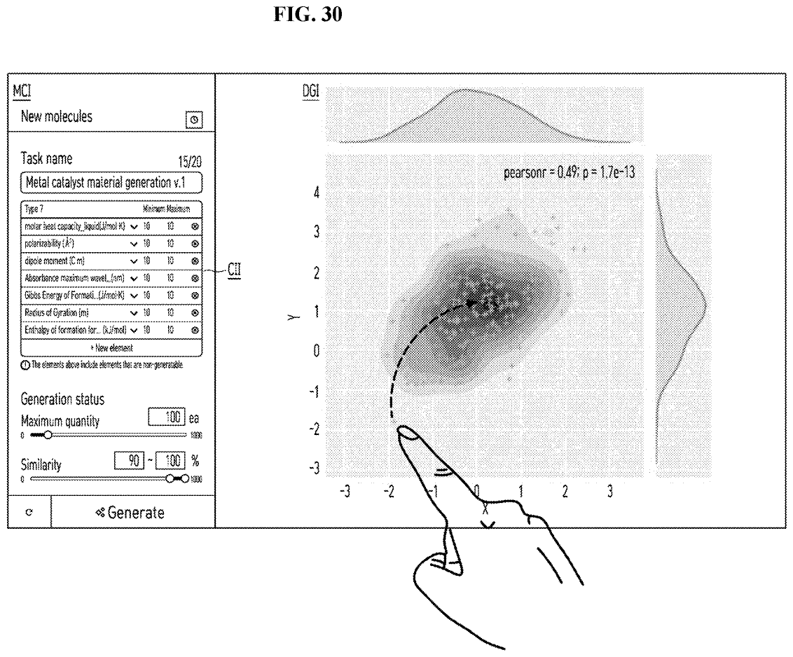

FIG. 30 illustrates an example for describing a physical property change interface according to an embodiment of the present disclosure.

In detail, referring to FIG. 30, in an embodiment, the computing system 1000 may provide a user interface MCI (hereinafter, a physical property change interface) capable of setting physical property change information in various manners.

Further, the computing system 1000 may obtain the above-described physical property change information based on user input based on the provided physical property change interface MCI.

In an embodiment, the computing system 1000 may provide a physical property change interface MCI including an interface DGI (hereinafter, a data density graph interface) for displaying density values estimated through the generation difficulty evaluation module GDM in a graph format.

In particular, the computing system 1000 may provide the physical property change interface MCI including the data density graph interface DGI for graphically displaying distributions of data points (i.e., physical property values) related to physical property input information as estimated by the generation difficulty evaluation module GDM.

In addition, in an embodiment, the computing system 1000 may obtain user input for setting specific physical properties and/or physical property values based on the provided data density graph interface DGI.

In an embodiment, the computing system 1000 may obtain user input (e.g., a drag input, etc.) for changing a position of a data point (i.e., a physical property value) on a graph (hereinafter, a data density graph) displayed through the data density graph interface DGI.

For example, the computing system 1000 may obtain user input for moving a first data point (i.e., a first physical property value) disposed in a low-density region on the data density graph to a high-density region on the data density graph.

As another example, the computing system 1000 may obtain user input (i.e., user input of removing the first data point from the data density graph) for moving the first data point (i.e., the first physical property value) disposed in the low-density region on the data density graph to remaining regions on the data density graph.

As described above, in an embodiment, the computing system 1000 may obtain physical property change information in response to user input for each data point (i.e., physical property value) visualized in the form of a graph image.

In particular, the computing system 1000 may obtain physical property change information based on an interface supporting easier and more intuitive user interaction.

In another embodiment, the computing system 1000 may provide a physical property change interface MCI including an interface CII (hereinafter, a change input interface) for setting physical property change information through predetermined text (e.g., characters and/or numbers) and/or selection input.

In addition, in an embodiment, the computing system 1000 may obtain user input for setting specific physical properties and/or physical property values based on predetermined text and/or selection input through the provided change input interface CII.

In an embodiment, the computing system 1000 may obtain a user's selection input (e.g., a first physical property name selection input, etc.) and/or a text input (e.g., a first physical property name text input, etc.) for setting physical properties to be removed, replaced, and/or added.

In addition, in an embodiment, the computing system 1000 may obtain a user's selection input (e.g., a first physical property value selection input, etc.) and/or a text input (e.g., a first physical property value text input, etc.) for setting physical property values to be changed (updated).

As such, in an embodiment, the computing system 1000 may obtain specific physical properties and/or physical property values with high accuracy based on concrete numerical values or explicit text through text and/or a selection input.

Accordingly, the computing system 1000 may obtain physical property change information that more precisely reflects user's needs.

Although the above-described embodiments have been described separately for the sake of effective description, according to embodiments, at least some of the above-described embodiments may be organically combined and operate together, and various other embodiments are possible.

In an embodiment, the computing system 1000 may provide the data density graph interface DGI and the change input interface CII in an organically combined manner, such that, according to a user's text and/or selection input, a first physical property is added, and physical property change information is obtained in which a physical property value of the first physical property is set according to a user's drag input for placing a first data point symbolizing the added first physical property on the data density graph.

As described above, in an embodiment, the computing system 1000 may obtain physical property change information specifying physical properties and/or physical property values to be set (changed) by a user by utilizing various types of user interfaces.

Accordingly, the computing system 1000 may enhance convenience and satisfaction of users employing the multi-tasking learning model MtLM, and may also easily enhance the usability thereof.

In addition, the computing system 1000 according to an embodiment of the present disclosure may provide an updated physical property guide MG based on the obtained physical property change information (S409).

Here, the updated physical property guide MG according to an embodiment may refer to a physical property guide MG based on physical property input information reflecting the above-described physical property change information (hereinafter, physical property update information).

In particular, in an embodiment, the updated physical property guide MG may refer to a physical property guide MG based on physical property update information reflecting newly set physical properties and/or physical property values according to user input.

In detail, in an embodiment, the computing system 1000 may generate physical property update information, which is physical property input information reflecting the obtained physical property change information.

In an embodiment, the computing system 1000 may generate physical property update information by applying changes according to the physical property change information to existing physical property input information.

In another embodiment, the computing system 1000 may generate new physical property input information based on the physical properties and/or physical property values according to the physical property change information, thereby generating the physical property update information.

In addition, in an embodiment, the computing system 1000 may execute the processes disclosed in above-described steps S403 and S405 based on the generated physical property update information.

In particular, the computing system 1000 may execute a process of calculating a validity index according to the physical property update information and generating and providing a physical property guide MG (i.e., an updated physical property guide MG) based on the calculated validity index.

In this regard, in an embodiment, the computing system 1000 may repeatedly execute the above-described process by generating the physical property update information whenever the physical property change information is obtained.

Accordingly, in an embodiment, the computing system 1000 may provide an updated physical property guide MG that analyzes molecular generation difficulty based on updated predetermined physical properties and/or physical property values whenever they are updated through user input, and provides a guideline in a direction to increase the validity thereof.

Accordingly, the computing system 1000 may enable a user to easily understand and modify the validity of the input values input, thereby effectively improving user experience in using the multi-tasking model guide provision service.

In addition, accordingly, the computing system 1000 may support identification and avoidance of abnormal input values, thereby enhancing the success rate and prediction accuracy of a molecular generation model, and increasing the reliability and stability thereof.

In addition, accordingly, the computing system 1000 may induce effective data supplementation and retraining, thereby contributing to continuous performance enhancement of the molecular generation model.

Accordingly, the computing system 1000 may provide a multi-tasking model that generates and outputs molecules in a form that further guarantees physical/scientific validity, thereby directly and significantly improving the quality of service processes such as experimental verification based thereon.

As described above, a method and a system for providing a guide supporting performance enhancement of a multi-tasking model according to an embodiment of the present disclosure may quantitatively evaluate validity of input data of the multi-tasking model and provide a guide according thereto, thereby inducing identification and avoidance of abnormal input values, and enhancing prediction performance and generation success rate of the multi-tasking model, thus improving reliability and accuracy thereof.

In addition, accordingly, a method and a system for providing a guide supporting performance enhancement of a multi-tasking model according to an embodiment of the present disclosure may support continuous enhancement of the multi-tasking model by inducing effective data supplementation and retraining to secure data diversity.

In addition, accordingly, a method and a system for providing a guide supporting performance enhancement of a multi-tasking model according to an embodiment of the present disclosure may easily secure physical/scientific validity for outputs of the multi-tasking model.

In addition, a method and a system for providing a guide supporting performance enhancement of a multi-tasking model according to an embodiment of the present disclosure may quantitatively evaluate validity of input data for the multi-tasking model using various deep learning algorithms, thereby promoting enhancement in evaluation accuracy and reliability through multifaceted analysis.

In addition, a method and a system for providing a guide supporting performance enhancement of a multi-tasking model according to an embodiment of the present disclosure may visualize and provide a guide based on quantitative evaluation results, thereby providing a more intuitive and clear guide to further enhance user understanding and satisfaction.

The embodiments according to the present disclosure described above may be implemented in the form of program instructions executable through various computer components and recorded on a computer-readable recording medium. The computer-readable recording medium may include program instructions, data files, data structures, and the like alone or in combination. The program instructions recorded in the computer-readable recording medium may be specially designed and configured for the present disclosure or may be known and available to those skilled in the field of computer software. Examples of the computer-readable recording medium include magnetic media such as a hard disk, a floppy disk and a magnetic tape, optical recording media such as a CD-ROM and a DVD, magneto-optical media such as a floptical disk, and hardware devices specially configured to store and execute program instructions such as a ROM, a RAM, a flash memory, and the like. Examples of program instructions include not only machine language codes such as those generated by a compiler, but also high-level language codes executable by a computer using an interpreter or the like. The hardware devices may be modified into one or more software modules to execute processing according to the present disclosure, and vice versa.

The specific implementations described herein are merely embodiments, and do not limit the scope of the present disclosure in any way. For the sake of brevity of the present document, descriptions of existing electronic components, control systems, software, and other functional aspects of the systems may be omitted. In addition, the line connections or connection members between the components illustrated in the drawings are intended to exemplarily represent functional connections and/or physical or circuit connections, and in actual devices, may be represented as various functional connections, physical connections, or circuit connections that are replaceable or additional. In addition, when there is no specific mention such as “essential”, “important”, or the like, a component may not be an essential component for the application of the present disclosure.

In addition, although the detailed description of the present disclosure has been provided with reference to embodiments of the present disclosure, those skilled in the art or those having ordinary knowledge in the art will understand that various modifications and changes may be made to the present disclosure without departing from the spirit and technical scope of the present disclosure as set forth in the claims below. Therefore, the technical scope of the present disclosure should not be limited to the contents in the detailed description of the present document, but should be determined by the claims.

The present disclosure relates to a method and a system for providing a guide supporting performance enhancement of a multi-tasking model, a method for sampling data for a general-purpose multi-tasking model, a method and a system for providing a general-purpose multi-tasking model including the same. The present disclosure is applicable to the artificial intelligence industry and thus has industrial applicability.

In one implementation, the target characteristics may include an optimal solution to be achieved by output data of the prediction process executed by the multi-tasking model.

In one implementation, the providing of the multi-tasking model may include providing a predetermined combination of physical properties according to the prediction process executed in a direction satisfying the target characteristics.

In one implementation, the prediction process may use the input information as input data, and use the predetermined physical property combination according to the input information as output data.

In one implementation, the prediction process may generate the predetermined physical property combination according to the input information based on geometric alignment in an integrated latent space.

According to an aspect of the present disclosure, provided is a system for providing a general-purpose multi-tasking model, the system comprising: at least one memory; and at least one processor configured to read at least one application stored in the memory and provide the general-purpose multi-tasking model, wherein instructions of the processor include instructions for executing: obtaining input information specifying predetermined domain characteristics; converting the obtained input information into low-dimensional latent variables that are data represented in a low-dimensional Gaussian space; obtaining sampled latent variables that are data obtained by sampling the converted low-dimensional latent variables; obtaining optimized latent variables that are data obtained by optimizing the obtained sampled latent variables through a genetic algorithm; restoring the obtained optimized latent variables into high-dimensional latent variables that are data represented in a high-dimensional space; providing the restored high-dimensional latent variables to a multi-tasking model; and providing the multi-tasking model.

A method and a system for providing a guide supporting performance enhancement of a multi-tasking model according to an embodiment of the present disclosure may quantitatively evaluate validity of input data of the multi-tasking model and provide a guide according thereto, thereby inducing identification and avoidance of abnormal input values, and enhancing prediction performance and generation success rate of the multi-tasking model, thus improving reliability and accuracy thereof.

In addition, accordingly, a method and a system for providing a guide supporting performance enhancement of a multi-tasking model according to an embodiment of the present disclosure may support continuous enhancement of the multi-tasking model by inducing effective data supplementation and retraining to secure data diversity.

In addition, accordingly, a method and a system for providing a guide supporting performance enhancement of a multi-tasking model according to an embodiment of the present disclosure may easily secure physical/scientific validity for outputs of the multi-tasking model.

In addition, a method and a system for providing a guide supporting performance enhancement of a multi-tasking model according to an embodiment of the present disclosure may quantitatively evaluate validity of input data for the multi-tasking model using various deep learning algorithms, thereby promoting enhancement in evaluation accuracy and reliability through multifaceted analysis.

In addition, a method and a system for providing a guide supporting performance enhancement of a multi-tasking model according to an embodiment of the present disclosure may visualize and provide a guide based on quantitative evaluation results, thereby providing a more intuitive and clear guide to further enhance user understanding and satisfaction.

In addition, a method for sampling data for a general-purpose multi-tasking model, and a method and a system for providing a general-purpose multi-tasking model including the same according to an embodiment of the present disclosure may efficiently search for and optimize data in a high-dimensional space through data sampling using a low-dimensional space, thereby reducing the calculation cost of a general-purpose multi-tasking model that handles large-scale data and directly improving the performance thereof.

In addition, a method for sampling data for a general-purpose multi-tasking model, and a method and a system for providing a general-purpose multi-tasking model including the same according to an embodiment of the present disclosure may automatically assign valid values for missing data during a data-dimensional restoration process, even when at least some of the data required for deep learning prediction are absent.

Accordingly, a method for sampling data for a general-purpose multi-tasking model, and a method and a system for providing a general-purpose multi-tasking model including the same according to an embodiment of the present disclosure may naturally combine values automatically assigned to missing data with the given data even without a separate data request for the missing data, thereby generating and providing physically/logically reasonable outputs.

Accordingly, a method for sampling data for a general-purpose multi-tasking model, and a method and a system for providing a general-purpose multi-tasking model including the same according to an embodiment of the present disclosure may further expand the applicability range of a general-purpose multi-tasking model that processes a large amount of various data.

In addition, a method for sampling data for a general-purpose multi-tasking model, and a method and a system for providing a general-purpose multi-tasking model including the same according to an embodiment of the present disclosure may execute transfer learning through geometric alignment in an integrated latent space for multi-tasks corresponding to a plurality of domains, thereby accurately predicting an integrated output satisfying needs of the plurality of domains.

In addition, a method for sampling data for a general-purpose multi-tasking model, and a method and a system for providing a general-purpose multi-tasking model including the same according to an embodiment of the present disclosure may simultaneously train various prediction tasks corresponding to a plurality of domains in the transfer learning process to achieve collective learning of not only individual principles of the respective domains but also correlations between the domains and a common principle for the entire domains, thereby expanding a model learning area and also expanding a prediction acceptance range for each domain.

Accordingly, a method for sampling data for a general-purpose multi-tasking model, and a method and a system for providing a general-purpose multi-tasking model including the same according to an embodiment of the present disclosure may directly enhance performance and quality of processing various multi-tasking tasks using the trained model.

In addition, a method for sampling data for a general-purpose multi-tasking model, and a method and a system for providing a general-purpose multi-tasking model including the same according to an embodiment of the present disclosure may implement mutual exchange of information by aligning geometric characteristics of the various prediction tasks, thereby easily supporting transfer of knowledge among mutually related data and enhancement of prediction performance resulted therefrom.

Furthermore, accordingly, a method for sampling data for a general-purpose multi-tasking model, and a method and a system for providing a general-purpose multi-tasking model including the same according to an embodiment of the present disclosure may increase resistance to unnecessary interference information and increase stability of the model.

In addition, a method for sampling data for a general-purpose multi-tasking model, and a method and a system for providing a general-purpose multi-tasking model including the same according to an embodiment of the present disclosure may secure data sets for training by utilizing source data from various sources, thereby increasing the diversity of data used for model training and allowing a model to learn more information to enhance learning performance.

In addition, a method for sampling data for a general-purpose multi-tasking model, and a method and a system for providing a general-purpose multi-tasking model including the same according to an embodiment of the present disclosure may predict a plurality of physical properties for a specific material by applying the multi-tasking learning model trained as described above to relationship predictions between a plurality of physical properties and materials, and provide a multi-tasking model capable of predicting a specific material satisfying a plurality of physical properties, thereby improving overall quality across related industries by providing a multi-tasking model that may be universally utilized for various materials.

A method for sampling data for a general-purpose multi-tasking model, and a method and a system for providing a general-purpose multi-tasking model including the same according to an embodiment of the present disclosure may transfer knowledge learned from a source task to a target task through transfer learning to solve data shortage problems, thereby providing a multi-tasking model that maintains high performance for multi-tasks even with small-scale data sets.

Therefore, a method for sampling data for a general-purpose multi-tasking model, and a method and a system for providing a general-purpose multi-tasking model including the same according to an embodiment of the present disclosure may expand the range of applicability to fields in which application of machine learning models had previously been difficult due to insufficient data or domain knowledge.

In addition, a method for sampling data for a general-purpose multi-tasking model, and a method and a system for providing a general-purpose multi-tasking model including the same according to an embodiment of the present disclosure may provide a specialized transfer learning technique that may be effectively applied to regression problems, thereby exhibiting high prediction performance even for complex regression problems such as molecular data sets.

In addition, a method for sampling data for a general-purpose multi-tasking model, and a method and a system for providing a general-purpose multi-tasking model including the same according to an embodiment of the present disclosure may optimize knowledge transfer between a source task and a target task through a Riemann geometric approach, thereby maintaining geometric consistency between tasks and improving the efficiency of transfer learning.

In addition, a method for sampling data for a general-purpose multi-tasking model, and a method and a system for providing a general-purpose multi-tasking model including the same according to an embodiment of the present disclosure may normalize various aspects of the model by combining multiple loss functions, thereby further improving generalization performance of the model.

Although certain embodiments and implementations have been described herein, other embodiments and modifications will be apparent from this description. Accordingly, the inventive concepts are not limited to such embodiments, but rather to the broader scope of the appended claims and various obvious modifications and equivalent arrangements as would be apparent to a person of ordinary skill in the art.

What is claimed is:

1. A method executed by a computer, the method comprising:
receiving, by at least one processor, first input information that specifies predetermined domain characteristics;
loading, by the at least one processor, at least one pre-trained artificial intelligence model stored in at least one memory based on the received first input information, the artificial intelligence model being trained using at least one algorithm among at least one density estimation algorithm, at least one anomaly detection algorithm, and at least one similarity determination algorithm;
ingesting, by the at least one processor, the first input information into the loaded at least one artificial intelligence model, and generating that quantitatively specifies a difficulty level associated with generating output data of at least one multi-tasking model based on the first input information;
generating, by the at least one processor, first guide information that specifies the predetermined domain characteristics configured to reduce the generation difficulty based on the generated validity index; and
manifesting the generated first guide information through at least one interface.

2. The method of claim 1, further comprising:
obtaining, by the at least one processor, second input information that specifies the predetermined domain characteristics;
obtaining, by the at least one processor, update information that replaces the first input information based on the obtained second input information; and
providing the first guide information according to the obtained update information.

3. The method of claim 2, wherein the generating of the validity index includes at least one of:
estimating a density value for the first input information based on the at least one density estimation algorithm, and calculating the validity index in proportion to the estimated density value;
detecting outliers in the first input information based on the at least one anomaly detection algorithm, estimating a density value for the first input information in inverse proportion to a number of detected outliers, and calculating the validity index in proportion to the estimated density value; and
calculating a similarity between the output data corresponding to the first input information and actual data based on the at least one similarity determination algorithm, and calculating the validity index in proportion to the calculated similarity.

4. The method of claim 2, wherein the predetermined domain characteristics include characteristics corresponding to each of a plurality of physical properties.

5. The method of claim 4, wherein the first guide information includes a physical property guide that specifies at least one of predetermined physical properties and characteristic values of the physical properties.

6. The method of claim 2, further comprising:
generating, by the at least one processor, second guide information that specifies a model training method configured to reduce the generation difficulty based on the generated validity index; and
providing the generated second guide information.

7. The method of claim 6, further comprising providing the second guide information according to the obtained update information.

8. The method of claim 2, wherein the obtaining of the second input information includes:
providing a data density graph interface that displays, in a graph format, a density value for the first input information derived during specification of the generation difficulty; and
obtaining the second input information in response to a user input made through the provided data density graph interface.

9. The method of claim 8, wherein the obtaining of the second input information includes obtaining the second input information in response to a user input that changes a position of a predetermined data point displayed on the data density graph interface.

10. A system comprising:
at least one memory; and
at least one processor configured to read at least one application stored in the at least one memory and provide a guide supporting performance enhancement of a multi-tasking model,
wherein the at least one processor is configured to execute instructions to:
receive, by the at least one processor, first input information specifying predetermined domain characteristics;
load, by the at least one processor, at least one pre-trained artificial intelligence model stored in the at least one memory based on the received first input information, the artificial intelligence model being trained using at least one algorithm among at least one density estimation algorithm, at least one anomaly detection algorithm, and at least one similarity determination algorithm;
ingest, by the at least one processor, the first input information into the loaded at least one artificial intelligence model, and generate a validity index that quantitatively specifies a difficulty in generating output data of at least one multi-tasking model according to the first input information;
generate, by the at least one processor, first guide information that specifies the predetermined domain characteristics configured to reduce the generation difficulty based on the generated validity index; and
manifest the generated first guide information through at least one interface.

11. A method executed by a computer, the method comprising:
receiving, by at least one processor of the computer, input information specifying predetermined domain characteristics;
converting, by the at least one processor, the received input information into low-dimensional latent variables represented in a low-dimensional Gaussian space;
obtaining, by the at least one processor, sampled latent variables by sampling the converted low-dimensional latent variables;
ingesting, by the at least one processor, the obtained sampled latent variables into at least one artificial intelligence model stored in at least one memory, and obtaining optimized latent variables that are sampled and optimized in the low-dimensional Gaussian space, the artificial intelligence model being trained using at least one genetic algorithm;
restoring, by the at least one processor, the obtained optimized latent variables into high-dimensional latent variables represented in a high-dimensional space;
ingesting, by the at least one processor, the restored high-dimensional latent variables into at least one multi-tasking model; and
manifesting, by the at least one processor, output data of the at least one multi-tasking model through at least one interface.

12. The method of claim 11, wherein the receiving of the input information specifying the domain characteristics includes obtaining N-dimensional input information specifying N domain characteristics, where N satisfies 1≤N<T,
wherein the converting of the obtained input information into the low-dimensional latent variables includes converting the obtained N-dimensional input information into low-dimensional latent variables in an L-dimensional Gaussian space, where L satisfies 1≤L<N<T, and
wherein the restoring of the obtained optimized latent variables into the high-dimensional latent variables includes restoring the L-dimensional optimized latent variables into high-dimensional latent variables in a-dimensional space, where T satisfies T≥2.

13. The method of claim 12, wherein the input information includes physical property input information that is N-dimensional data specifying characteristic values for each of N physical properties, where N satisfies 1≤N<T.

14. The method of claim 13, wherein the obtaining of the sampled latent variables includes sampling the low-dimensional latent variables based on the physical property input information to obtain the sampled latent variables representing a predetermined combination of physical properties.

15. The method of claim 14, wherein the obtaining of the optimized latent variables includes optimizing the sampled latent variables, based on the at least one genetic algorithm, so as to satisfy the characteristic values for each of the physical properties specified in the physical property input information.

16. The method of claim 15, wherein the obtaining of the optimized latent variables further includes optimizing, among T physical properties included in a T-dimensional physical property combination, where T satisfies T≥2, the N physical properties specified in the physical property input information so as to satisfy the respective characteristic values corresponding thereto.

17. The method of claim 16, wherein the restoring of the obtained optimized latent variables into the high-dimensional latent variables further includes converting the L-dimensional optimized latent variables into the T-dimensional, high-dimensional latent variables while maintaining correlations among the T physical properties.

18. The method of claim 17, wherein the high-dimensional latent variables represent the physical property combination having a physical feasibility equal to or greater than a predetermined criterion while satisfying the characteristic values for each of the physical properties specified in the physical property input information.

19. The method of claim 11, wherein the ingesting of the high-dimensional latent variables to the at least one multi-tasking model includes providing the high-dimensional latent variables as target characteristics of a prediction process executed by the multi-tasking model, and
wherein the target characteristics include an optimal solution to be achieved by output data of the prediction process executed by the multi-tasking model.

20. The method of claim 19, wherein the manifesting of the output data of the at least one multi-tasking model through at least one interface includes providing a predetermined combination of physical properties according to the prediction process executed in a manner that satisfies the target characteristics, and
wherein the prediction process uses the input information as input data, uses the predetermined physical property combination according to the input information as output data, and generates the predetermined physical property combination based on geometric alignment in an integrated latent space.
